SEQUENCE LISTING (247 sequences)

SEQ ID NO: 1 - protein, 35 aa, Artificial Sequence
  Based on Homo sapiens sequence
PRKGGRRRRAWGHRRHRHHHRRRRRRRPRKRLRLR

SEQ ID NO: 2 - protein, 35 aa, Artificial Sequence
  Based on Homo sapiens sequence
PRKGGRRRRAWGKRRKRKKKRRRRRRRPRKRLRLR

SEQ ID NO: 3 - protein, 35 aa, Artificial Sequence
  Based on Homo sapiens sequence
PRKGGRRRRAWGRRRHRHKKRRRRRRRPRKRLRLR

SEQ ID NO: 4 - protein, 35 aa, Artificial Sequence
  Based on Homo sapiens sequence
PRKGGRRRRAWGRRRHRRHHRRRRRRRPRKRLRLR

SEQ ID NO: 5 - protein, 35 aa, Artificial Sequence
  Based on Homo sapiens sequence
PRKGGRRRRAWGRRRKRRHHRRRRRRRPRKRLRLR

SEQ ID NO: 6 - protein, 35 aa, Artificial Sequence
  Based on Homo sapiens sequence
PRKGGRRRRAWGRRRKRRHKRRRRRRRPRKRLRLR

SEQ ID NO: 7 - protein, 35 aa, Artificial Sequence
  Based on Homo sapiens sequence
PRKGGRRRRAWGRRRKRRKKRRRRRRRPRKRLRLR

SEQ ID NO: 8 - protein, 35 aa, Artificial Sequence
  Based on Homo sapiens sequence
PRKGGRRRRAWGRRRRRRHKRRRRRRRPRKRLRLR

SEQ ID NO: 9 - protein, 35 aa, Artificial Sequence
  Based on Homo sapiens sequence
PRKGGRRRRAWGRRRRRRRKRRRRRRRPRKRLRLR

SEQ ID NO: 10 - protein, 35 aa, Artificial Sequence
  Based on Homo sapiens sequence
PRKGGRRRRAWGRRRRRRRRRRRRRRRPRKRLRLR

SEQ ID NO: 11 - protein, 35 aa, Artificial Sequence
  Based on Homo sapiens sequence
  misc_feature (1)..(1): Xaa is D-Arginine
XPRKGGRRRRAWGRRRRRRRRRRRRRRAPRKRLTL

SEQ ID NO: 12 - protein, 35 aa, Artificial Sequence
  Based on Homo sapiens sequence
PRKGGSRRRAWGNQRYARLIRQAIESAPEKRLTLA

SEQ ID NO: 13 - protein, 35 aa, Artificial Sequence
  Based on Homo sapiens sequence
PRKGGRRRRAWGHRRHRHHKRRRRRRRPRKRLRLR

SEQ ID NO: 14 - protein, 35 aa, Artificial Sequence
  Based on Homo sapiens sequence
PRKGGRRRRAWGHRRHRHHRRRRRRRRPRKRLRLR

SEQ ID NO: 15 - protein, 35 aa, Artificial Sequence
  Based on Homo sapiens sequence
PRKGGRRRRAWGHRRHRHKHRRRRRRRPRKRLRLR

SEQ ID NO: 16 - protein, 35 aa, Artificial Sequence
  Based on Homo sapiens sequence
PRKGGRRRRAWGHRRHRHKKRRRRRRRPRKRLRLR

SEQ ID NO: 17 - protein, 35 aa, Artificial Sequence
  Based on Homo sapiens sequence
PRKGGRRRRAWGHRRHRHKRRRRRRRRPRKRLRLR

SEQ ID NO: 18 - protein, 35 aa, Artificial Sequence
  Based on Homo sapiens sequence
PRKGGRRRRAWGHRRHRHRHRRRRRRRPRKRLRLR

SEQ ID NO: 19 - protein, 35 aa, Artificial Sequence
  Based on Homo sapiens sequence
PRKGGRRRRAWGHRRHRHRKRRRRRRRPRKRLRLR

SEQ ID NO: 20 - protein, 35 aa, Artificial Sequence
  Based on Homo sapiens sequence
PRKGGRRRRAWGHRRHRHRRRRRRRRRPRKRLRLR

SEQ ID NO: 21 - protein, 35 aa, Artificial Sequence
  Based on Homo sapiens sequence
PRKGGRRRRAWGHRRHRKHHRRRRRRRPRKRLRLR

SEQ ID NO: 22 - protein, 35 aa, Artificial Sequence
  Based on Homo sapiens sequence
PRKGGRRRRAWGHRRHRKHKRRRRRRRPRKRLRLR

SEQ ID NO: 23 - protein, 35 aa, Artificial Sequence
  Based on Homo sapiens sequence
PRKGGRRRRAWGHRRHRKHRRRRRRRRPRKRLRLR

SEQ ID NO: 24 - protein, 35 aa, Artificial Sequence
  Based on Homo sapiens sequence
PRKGGRRRRAWGHRRHRKKHRRRRRRRPRKRLRLR

SEQ ID NO: 25 - protein, 35 aa, Artificial Sequence
  Based on Homo sapiens sequence
PRKGGRRRRAWGHRRHRKKKRRRRRRRPRKRLRLR

SEQ ID NO: 26 - protein, 35 aa, Artificial Sequence
  Based on Homo sapiens sequence
PRKGGRRRRAWGHRRHRKKRRRRRRRRPRKRLRLR

SEQ ID NO: 27 - protein, 35 aa, Artificial Sequence
  Based on Homo sapiens sequence
PRKGGRRRRAWGHRRHRKRHRRRRRRRPRKRLRLR

SEQ ID NO: 28 - protein, 35 aa, Artificial Sequence
  Based on Homo sapiens sequence
PRKGGRRRRAWGHRRHRKRKRRRRRRRPRKRLRLR

SEQ ID NO: 29 - protein, 35 aa, Artificial Sequence
  Based on Homo sapiens sequence
PRKGGRRRRAWGHRRHRKRRRRRRRRRPRKRLRLR

SEQ ID NO: 30 - protein, 35 aa, Artificial Sequence
  Based on Homo sapiens sequence
PRKGGRRRRAWGHRRHRRHHRRRRRRRPRKRLRLR

SEQ ID NO: 31 - protein, 35 aa, Artificial Sequence
  Based on Homo sapiens sequence
PRKGGRRRRAWGHRRHRRHKRRRRRRRPRKRLRLR

SEQ ID NO: 32 - protein, 35 aa, Artificial Sequence
  Based on Homo sapiens sequence
PRKGGRRRRAWGHRRHRRHRRRRRRRRPRKRLRLR

SEQ ID NO: 33 - protein, 35 aa, Artificial Sequence
  Based on Homo sapiens sequence
PRKGGRRRRAWGHRRHRRKHRRRRRRRPRKRLRLR

SEQ ID NO: 34 - protein, 35 aa, Artificial Sequence
  Based on Homo sapiens sequence
PRKGGRRRRAWGHRRHRRKKRRRRRRRPRKRLRLR

SEQ ID NO: 35 - protein, 35 aa, Artificial Sequence
  Based on Homo sapiens sequence
PRKGGRRRRAWGHRRHRRKRRRRRRRRPRKRLRLR

SEQ ID NO: 36 - protein, 35 aa, Artificial Sequence
  Based on Homo sapiens sequence
PRKGGRRRRAWGHRRHRRRHRRRRRRRPRKRLRLR

SEQ ID NO: 37 - protein, 35 aa, Artificial Sequence
  Based on Homo sapiens sequence
PRKGGRRRRAWGHRRHRRRKRRRRRRRPRKRLRLR

SEQ ID NO: 38 - protein, 35 aa, Artificial Sequence
  Based on Homo sapiens sequence
PRKGGRRRRAWGHRRHRRRRRRRRRRRPRKRLRLR

SEQ ID NO: 39 - protein, 35 aa, Artificial Sequence
  Based on Homo sapiens sequence
PRKGGRRRRAWGHRRKRHHHRRRRRRRPRKRLRLR

SEQ ID NO: 40 - protein, 35 aa, Artificial Sequence
  Based on Homo sapiens sequence
PRKGGRRRRAWGHRRKRHHKRRRRRRRPRKRLRLR

SEQ ID NO: 41 - protein, 35 aa, Artificial Sequence
  Based on Homo sapiens sequence
PRKGGRRRRAWGHRRKRHHRRRRRRRRPRKRLRLR

SEQ ID NO: 42 - protein, 35 aa, Artificial Sequence
  Based on Homo sapiens sequence
PRKGGRRRRAWGHRRKRHKHRRRRRRRPRKRLRLR

SEQ ID NO: 43 - protein, 35 aa, Artificial Sequence
  Based on Homo sapiens sequence
PRKGGRRRRAWGHRRKRHKKRRRRRRRPRKRLRLR

SEQ ID NO: 44 - protein, 35 aa, Artificial Sequence
  Based on Homo sapiens sequence
PRKGGRRRRAWGHRRKRHKRRRRRRRRPRKRLRLR

SEQ ID NO: 45 - protein, 35 aa, Artificial Sequence
  Based on Homo sapiens sequence
PRKGGRRRRAWGHRRKRHRHRRRRRRRPRKRLRLR

SEQ ID NO: 46 - protein, 35 aa, Artificial Sequence
  Based on Homo sapiens sequence
PRKGGRRRRAWGHRRKRHRKRRRRRRRPRKRLRLR

SEQ ID NO: 47 - protein, 35 aa, Artificial Sequence
  Based on Homo sapiens sequence
PRKGGRRRRAWGHRRKRHRRRRRRRRRPRKRLRLR

SEQ ID NO: 48 - protein, 35 aa, Artificial Sequence
  Based on Homo sapiens sequence
PRKGGRRRRAWGHRRKRKHHRRRRRRRPRKRLRLR

SEQ ID NO: 49 - protein, 35 aa, Artificial Sequence
  Based on Homo sapiens sequence
PRKGGRRRRAWGHRRKRKHKRRRRRRRPRKRLRLR

SEQ ID NO: 50 - protein, 35 aa, Artificial Sequence
  Based on Homo sapiens sequence
PRKGGRRRRAWGHRRKRKHRRRRRRRRPRKRLRLR

SEQ ID NO: 51 - protein, 35 aa, Artificial Sequence
  Based on Homo sapiens sequence
PRKGGRRRRAWGHRRKRKKHRRRRRRRPRKRLRLR

SEQ ID NO: 52 - protein, 35 aa, Artificial Sequence
  Based on Homo sapiens sequence
PRKGGRRRRAWGHRRKRKKKRRRRRRRPRKRLRLR

SEQ ID NO: 53 - protein, 35 aa, Artificial Sequence
  Based on Homo sapiens sequence
PRKGGRRRRAWGHRRKRKKRRRRRRRRPRKRLRLR

SEQ ID NO: 54 - protein, 35 aa, Artificial Sequence
  Based on Homo sapiens sequence
PRKGGRRRRAWGHRRKRKRHRRRRRRRPRKRLRLR

SEQ ID NO: 55 - protein, 35 aa, Artificial Sequence
  Based on Homo sapiens sequence
PRKGGRRRRAWGHRRKRKRKRRRRRRRPRKRLRLR

SEQ ID NO: 56 - protein, 35 aa, Artificial Sequence
  Based on Homo sapiens sequence
PRKGGRRRRAWGHRRKRKRRRRRRRRRPRKRLRLR

SEQ ID NO: 57 - protein, 35 aa, Artificial Sequence
  Based on Homo sapiens sequence
PRKGGRRRRAWGHRRKRRHHRRRRRRRPRKRLRLR

SEQ ID NO: 58 - protein, 35 aa, Artificial Sequence
  Based on Homo sapiens sequence
PRKGGRRRRAWGHRRKRRHKRRRRRRRPRKRLRLR

SEQ ID NO: 59 - protein, 35 aa, Artificial Sequence
  Based on Homo sapiens sequence
PRKGGRRRRAWGHRRKRRHRRRRRRRRPRKRLRLR

SEQ ID NO: 60 - protein, 35 aa, Artificial Sequence
  Based on Homo sapiens sequence
PRKGGRRRRAWGHRRKRRKHRRRRRRRPRKRLRLR

SEQ ID NO: 61 - protein, 35 aa, Artificial Sequence
  Based on Homo sapiens sequence
PRKGGRRRRAWGHRRKRRKKRRRRRRRPRKRLRLR

SEQ ID NO: 62 - protein, 35 aa, Artificial Sequence
  Based on Homo sapiens sequence
PRKGGRRRRAWGHRRKRRKRRRRRRRRPRKRLRLR

SEQ ID NO: 63 - protein, 35 aa, Artificial Sequence
  Based on Homo sapiens sequence
PRKGGRRRRAWGHRRKRRRHRRRRRRRPRKRLRLR

SEQ ID NO: 64 - protein, 35 aa, Artificial Sequence
  Based on Homo sapiens sequence
PRKGGRRRRAWGHRRKRRRKRRRRRRRPRKRLRLR

SEQ ID NO: 65 - protein, 35 aa, Artificial Sequence
  Based on Homo sapiens sequence
PRKGGRRRRAWGHRRKRRRRRRRRRRRPRKRLRLR

SEQ ID NO: 66 - protein, 35 aa, Artificial Sequence
  Based on Homo sapiens sequence
PRKGGRRRRAWGHRRRRHHHRRRRRRRPRKRLRLR

SEQ ID NO: 67 - protein, 35 aa, Artificial Sequence
  Based on Homo sapiens sequence
PRKGGRRRRAWGHRRRRHHKRRRRRRRPRKRLRLR

SEQ ID NO: 68 - protein, 35 aa, Artificial Sequence
  Based on Homo sapiens sequence
PRKGGRRRRAWGHRRRRHHRRRRRRRRPRKRLRLR

SEQ ID NO: 69 - protein, 35 aa, Artificial Sequence
  Based on Homo sapiens sequence
PRKGGRRRRAWGHRRRRHKHRRRRRRRPRKRLRLR

SEQ ID NO: 70 - protein, 35 aa, Artificial Sequence
  Based on Homo sapiens sequence
PRKGGRRRRAWGHRRRRHKKRRRRRRRPRKRLRLR

SEQ ID NO: 71 - protein, 35 aa, Artificial Sequence
  Based on Homo sapiens sequence
PRKGGRRRRAWGHRRRRHKRRRRRRRRPRKRLRLR

SEQ ID NO: 72 - protein, 35 aa, Artificial Sequence
  Based on Homo sapiens sequence
PRKGGRRRRAWGHRRRRHRHRRRRRRRPRKRLRLR

SEQ ID NO: 73 - protein, 35 aa, Artificial Sequence
  Based on Homo sapiens sequence
PRKGGRRRRAWGHRRRRHRKRRRRRRRPRKRLRLR

SEQ ID NO: 74 - protein, 35 aa, Artificial Sequence
  Based on Homo sapiens sequence
PRKGGRRRRAWGHRRRRHRRRRRRRRRPRKRLRLR

SEQ ID NO: 75 - protein, 35 aa, Artificial Sequence
  Based on Homo sapiens sequence
PRKGGRRRRAWGHRRRRKHHRRRRRRRPRKRLRLR

SEQ ID NO: 76 - protein, 35 aa, Artificial Sequence
  Based on Homo sapiens sequence
PRKGGRRRRAWGHRRRRKHKRRRRRRRPRKRLRLR

SEQ ID NO: 77 - protein, 35 aa, Artificial Sequence
  Based on Homo sapiens sequence
PRKGGRRRRAWGHRRRRKHRRRRRRRRPRKRLRLR

SEQ ID NO: 78 - protein, 35 aa, Artificial Sequence
  Based on Homo sapiens sequence
PRKGGRRRRAWGHRRRRKKHRRRRRRRPRKRLRLR

SEQ ID NO: 79 - protein, 35 aa, Artificial Sequence
  Based on Homo sapiens sequence
PRKGGRRRRAWGHRRRRKKKRRRRRRRPRKRLRLR

SEQ ID NO: 80 - protein, 35 aa, Artificial Sequence
  Based on Homo sapiens sequence
PRKGGRRRRAWGHRRRRKKRRRRRRRRPRKRLRLR

SEQ ID NO: 81 - protein, 35 aa, Artificial Sequence
  Based on Homo sapiens sequence
PRKGGRRRRAWGHRRRRKRHRRRRRRRPRKRLRLR

SEQ ID NO: 82 - protein, 35 aa, Artificial Sequence
  Based on Homo sapiens sequence
PRKGGRRRRAWGHRRRRKRKRRRRRRRPRKRLRLR

SEQ ID NO: 83 - protein, 35 aa, Artificial Sequence
  Based on Homo sapiens sequence
PRKGGRRRRAWGHRRRRKRRRRRRRRRPRKRLRLR

SEQ ID NO: 84 - protein, 35 aa, Artificial Sequence
  Based on Homo sapiens sequence
PRKGGRRRRAWGHRRRRRHHRRRRRRRPRKRLRLR

SEQ ID NO: 85 - protein, 35 aa, Artificial Sequence
  Based on Homo sapiens sequence
PRKGGRRRRAWGHRRRRRHKRRRRRRRPRKRLRLR

SEQ ID NO: 86 - protein, 35 aa, Artificial Sequence
  Based on Homo sapiens sequence
PRKGGRRRRAWGHRRRRRHRRRRRRRRPRKRLRLR

SEQ ID NO: 87 - protein, 35 aa, Artificial Sequence
  Based on Homo sapiens sequence
PRKGGRRRRAWGHRRRRRKHRRRRRRRPRKRLRLR

SEQ ID NO: 88 - protein, 35 aa, Artificial Sequence
  Based on Homo sapiens sequence
PRKGGRRRRAWGHRRRRRKKRRRRRRRPRKRLRLR

SEQ ID NO: 89 - protein, 35 aa, Artificial Sequence
  Based on Homo sapiens sequence
PRKGGRRRRAWGHRRRRRKRRRRRRRRPRKRLRLR

SEQ ID NO: 90 - protein, 35 aa, Artificial Sequence
  Based on Homo sapiens sequence
PRKGGRRRRAWGHRRRRRRHRRRRRRRPRKRLRLR

SEQ ID NO: 91 - protein, 35 aa, Artificial Sequence
  Based on Homo sapiens sequence
PRKGGRRRRAWGHRRRRRRKRRRRRRRPRKRLRLR

SEQ ID NO: 92 - protein, 35 aa, Artificial Sequence
  Based on Homo sapiens sequence
PRKGGRRRRAWGHRRRRRRRRRRRRRRPRKRLRLR

SEQ ID NO: 93 - protein, 35 aa, Artificial Sequence
  Based on Homo sapiens sequence
PRKGGRRRRAWGKRRHRHHHRRRRRRRPRKRLRLR

SEQ ID NO: 94 - protein, 35 aa, Artificial Sequence
  Based on Homo sapiens sequence
PRKGGRRRRAWGKRRHRHHKRRRRRRRPRKRLRLR

SEQ ID NO: 95 - protein, 35 aa, Artificial Sequence
  Based on Homo sapiens sequence
PRKGGRRRRAWGKRRHRHHRRRRRRRRPRKRLRLR

SEQ ID NO: 96 - protein, 35 aa, Artificial Sequence
  Based on Homo sapiens sequence
PRKGGRRRRAWGKRRHRHKHRRRRRRRPRKRLRLR

SEQ ID NO: 97 - protein, 35 aa, Artificial Sequence
  Based on Homo sapiens sequence
PRKGGRRRRAWGKRRHRHKKRRRRRRRPRKRLRLR

SEQ ID NO: 98 - protein, 35 aa, Artificial Sequence
  Based on Homo sapiens sequence
PRKGGRRRRAWGKRRHRHKRRRRRRRRPRKRLRLR

SEQ ID NO: 99 - protein, 35 aa, Artificial Sequence
  Based on Homo sapiens sequence
PRKGGRRRRAWGKRRHRHRHRRRRRRRPRKRLRLR

SEQ ID NO: 100 - protein, 35 aa, Artificial Sequence
  Based on Homo sapiens sequence
PRKGGRRRRAWGKRRHRHRKRRRRRRRPRKRLRLR

SEQ ID NO: 101 - protein, 35 aa, Artificial Sequence
  Based on Homo sapiens sequence
PRKGGRRRRAWGKRRHRHRRRRRRRRRPRKRLRLR

SEQ ID NO: 102 - protein, 35 aa, Artificial Sequence
  Based on Homo sapiens sequence
PRKGGRRRRAWGKRRHRKHHRRRRRRRPRKRLRLR

SEQ ID NO: 103 - protein, 35 aa, Artificial Sequence
  Based on Homo sapiens sequence
PRKGGRRRRAWGKRRHRKHKRRRRRRRPRKRLRLR

SEQ ID NO: 104 - protein, 35 aa, Artificial Sequence
  Based on Homo sapiens sequence
PRKGGRRRRAWGKRRHRKHRRRRRRRRPRKRLRLR

SEQ ID NO: 105 - protein, 35 aa, Artificial Sequence
  Based on Homo sapiens sequence
PRKGGRRRRAWGKRRHRKKHRRRRRRRPRKRLRLR

SEQ ID NO: 106 - protein, 35 aa, Artificial Sequence
  Based on Homo sapiens sequence
PRKGGRRRRAWGKRRHRKKKRRRRRRRPRKRLRLR

SEQ ID NO: 107 - protein, 35 aa, Artificial Sequence
  Based on Homo sapiens sequence
PRKGGRRRRAWGKRRHRKKRRRRRRRRPRKRLRLR

SEQ ID NO: 108 - protein, 35 aa, Artificial Sequence
  Based on Homo sapiens sequence
PRKGGRRRRAWGKRRHRKRHRRRRRRRPRKRLRLR

SEQ ID NO: 109 - protein, 35 aa, Artificial Sequence
  Based on Homo sapiens sequence
PRKGGRRRRAWGKRRHRKRKRRRRRRRPRKRLRLR

SEQ ID NO: 110 - protein, 35 aa, Artificial Sequence
  Based on Homo sapiens sequence
PRKGGRRRRAWGKRRHRKRRRRRRRRRPRKRLRLR

SEQ ID NO: 111 - protein, 35 aa, Artificial Sequence
  Based on Homo sapiens sequence
PRKGGRRRRAWGKRRHRRHHRRRRRRRPRKRLRLR

SEQ ID NO: 112 - protein, 35 aa, Artificial Sequence
  Based on Homo sapiens sequence
PRKGGRRRRAWGKRRHRRHKRRRRRRRPRKRLRLR

SEQ ID NO: 113 - protein, 35 aa, Artificial Sequence
  Based on Homo sapiens sequence
PRKGGRRRRAWGKRRHRRHRRRRRRRRPRKRLRLR

SEQ ID NO: 114 - protein, 35 aa, Artificial Sequence
  Based on Homo sapiens sequence
PRKGGRRRRAWGKRRHRRKHRRRRRRRPRKRLRLR

SEQ ID NO: 115 - protein, 35 aa, Artificial Sequence
  Based on Homo sapiens sequence
PRKGGRRRRAWGKRRHRRKKRRRRRRRPRKRLRLR

SEQ ID NO: 116 - protein, 35 aa, Artificial Sequence
  Based on Homo sapiens sequence
PRKGGRRRRAWGKRRHRRKRRRRRRRRPRKRLRLR

SEQ ID NO: 117 - protein, 35 aa, Artificial Sequence
  Based on Homo sapiens sequence
PRKGGRRRRAWGKRRHRRRHRRRRRRRPRKRLRLR

SEQ ID NO: 118 - protein, 35 aa, Artificial Sequence
  Based on Homo sapiens sequence
PRKGGRRRRAWGKRRHRRRKRRRRRRRPRKRLRLR

SEQ ID NO: 119 - protein, 35 aa, Artificial Sequence
  Based on Homo sapiens sequence
PRKGGRRRRAWGKRRHRRRRRRRRRRRPRKRLRLR

SEQ ID NO: 120 - protein, 35 aa, Artificial Sequence
  Based on Homo sapiens sequence
PRKGGRRRRAWGKRRKRHHHRRRRRRRPRKRLRLR

SEQ ID NO: 121 - protein, 35 aa, Artificial Sequence
  Based on Homo sapiens sequence
PRKGGRRRRAWGKRRKRHHKRRRRRRRPRKRLRLR

SEQ ID NO: 122 - protein, 35 aa, Artificial Sequence
  Based on Homo sapiens sequence
PRKGGRRRRAWGKRRKRHHRRRRRRRRPRKRLRLR

SEQ ID NO: 123 - protein, 35 aa, Artificial Sequence
  Based on Homo sapiens sequence
PRKGGRRRRAWGKRRKRHKHRRRRRRRPRKRLRLR

SEQ ID NO: 124 - protein, 35 aa, Artificial Sequence
  Based on Homo sapiens sequence
PRKGGRRRRAWGKRRKRHKKRRRRRRRPRKRLRLR

SEQ ID NO: 125 - protein, 35 aa, Artificial Sequence
  Based on Homo sapiens sequence
PRKGGRRRRAWGKRRKRHKRRRRRRRRPRKRLRLR

SEQ ID NO: 126 - protein, 35 aa, Artificial Sequence
  Based on Homo sapiens sequence
PRKGGRRRRAWGKRRKRHRHRRRRRRRPRKRLRLR

SEQ ID NO: 127 - protein, 35 aa, Artificial Sequence
  Based on Homo sapiens sequence
PRKGGRRRRAWGKRRKRHRKRRRRRRRPRKRLRLR

SEQ ID NO: 128 - protein, 35 aa, Artificial Sequence
  Based on Homo sapiens sequence
PRKGGRRRRAWGKRRKRHRRRRRRRRRPRKRLRLR

SEQ ID NO: 129 - protein, 35 aa, Artificial Sequence
  Based on Homo sapiens sequence
PRKGGRRRRAWGKRRKRKHHRRRRRRRPRKRLRLR

SEQ ID NO: 130 - protein, 35 aa, Artificial Sequence
  Based on Homo sapiens sequence
PRKGGRRRRAWGKRRKRKHKRRRRRRRPRKRLRLR

SEQ ID NO: 131 - protein, 35 aa, Artificial Sequence
  Based on Homo sapiens sequence
PRKGGRRRRAWGKRRKRKHRRRRRRRRPRKRLRLR

SEQ ID NO: 132 - protein, 35 aa, Artificial Sequence
  Based on Homo sapiens sequence
PRKGGRRRRAWGKRRKRKKHRRRRRRRPRKRLRLR

SEQ ID NO: 133 - protein, 35 aa, Artificial Sequence
  Based on Homo sapiens sequence
PRKGGRRRRAWGKRRKRKKRRRRRRRRPRKRLRLR

SEQ ID NO: 134 - protein, 35 aa, Artificial Sequence
  Based on Homo sapiens sequence
PRKGGRRRRAWGKRRKRKRHRRRRRRRPRKRLRLR

SEQ ID NO: 135 - protein, 35 aa, Artificial Sequence
  Based on Homo sapiens sequence
PRKGGRRRRAWGKRRKRKRKRRRRRRRPRKRLRLR

SEQ ID NO: 136 - protein, 35 aa, Artificial Sequence
  Based on Homo sapiens sequence
PRKGGRRRRAWGKRRKRKRRRRRRRRRPRKRLRLR

SEQ ID NO: 137 - protein, 35 aa, Artificial Sequence
  Based on Homo sapiens sequence
PRKGGRRRRAWGKRRKRRHHRRRRRRRPRKRLRLR

SEQ ID NO: 138 - protein, 35 aa, Artificial Sequence
  Based on Homo sapiens sequence
PRKGGRRRRAWGKRRKRRHKRRRRRRRPRKRLRLR

SEQ ID NO: 139 - protein, 35 aa, Artificial Sequence
  Based on Homo sapiens sequence
PRKGGRRRRAWGKRRKRRHRRRRRRRRPRKRLRLR

SEQ ID NO: 140 - protein, 35 aa, Artificial Sequence
  Based on Homo sapiens sequence
PRKGGRRRRAWGKRRKRRKHRRRRRRRPRKRLRLR

SEQ ID NO: 141 - protein, 35 aa, Artificial Sequence
  Based on Homo sapiens sequence
PRKGGRRRRAWGKRRKRRKKRRRRRRRPRKRLRLR

SEQ ID NO: 142 - protein, 35 aa, Artificial Sequence
  Based on Homo sapiens sequence
PRKGGRRRRAWGKRRKRRKRRRRRRRRPRKRLRLR

SEQ ID NO: 143 - protein, 35 aa, Artificial Sequence
  Based on Homo sapiens sequence
PRKGGRRRRAWGKRRKRRRHRRRRRRRPRKRLRLR

SEQ ID NO: 144 - protein, 35 aa, Artificial Sequence
  Based on Homo sapiens sequence
PRKGGRRRRAWGKRRKRRRKRRRRRRRPRKRLRLR

SEQ ID NO: 145 - protein, 35 aa, Artificial Sequence
  Based on Homo sapiens sequence
PRKGGRRRRAWGKRRKRRRRRRRRRRRPRKRLRLR

SEQ ID NO: 146 - protein, 35 aa, Artificial Sequence
  Based on Homo sapiens sequence
PRKGGRRRRAWGKRRRRHHHRRRRRRRPRKRLRLR

SEQ ID NO: 147 - protein, 35 aa, Artificial Sequence
  Based on Homo sapiens sequence
PRKGGRRRRAWGKRRRRHHKRRRRRRRPRKRLRLR

SEQ ID NO: 148 - protein, 35 aa, Artificial Sequence
  Based on Homo sapiens sequence
PRKGGRRRRAWGKRRRRHHRRRRRRRRPRKRLRLR

SEQ ID NO: 149 - protein, 35 aa, Artificial Sequence
  Based on Homo sapiens sequence
PRKGGRRRRAWGKRRRRHKHRRRRRRRPRKRLRLR

SEQ ID NO: 150 - protein, 35 aa, Artificial Sequence
  Based on Homo sapiens sequence
PRKGGRRRRAWGKRRRRHKKRRRRRRRPRKRLRLR

SEQ ID NO: 151 - protein, 35 aa, Artificial Sequence
  Based on Homo sapiens sequence
PRKGGRRRRAWGKRRRRHKRRRRRRRRPRKRLRLR

SEQ ID NO: 152 - protein, 35 aa, Artificial Sequence
  Based on Homo sapiens sequence
PRKGGRRRRAWGKRRRRHRHRRRRRRRPRKRLRLR

SEQ ID NO: 153 - protein, 35 aa, Artificial Sequence
  Based on Homo sapiens sequence
PRKGGRRRRAWGKRRRRHRKRRRRRRRPRKRLRLR

SEQ ID NO: 154 - protein, 35 aa, Artificial Sequence
  Based on Homo sapiens sequence
PRKGGRRRRAWGKRRRRHRRRRRRRRRPRKRLRLR

SEQ ID NO: 155 - protein, 35 aa, Artificial Sequence
  Based on Homo sapiens sequence
PRKGGRRRRAWGKRRRRKHHRRRRRRRPRKRLRLR

SEQ ID NO: 156 - protein, 35 aa, Artificial Sequence
  Based on Homo sapiens sequence
PRKGGRRRRAWGKRRRRKHKRRRRRRRPRKRLRLR

SEQ ID NO: 157 - protein, 35 aa, Artificial Sequence
  Based on Homo sapiens sequence
PRKGGRRRRAWGKRRRRKHRRRRRRRRPRKRLRLR

SEQ ID NO: 158 - protein, 35 aa, Artificial Sequence
  Based on Homo sapiens sequence
PRKGGRRRRAWGKRRRRKKHRRRRRRRPRKRLRLR

SEQ ID NO: 159 - protein, 35 aa, Artificial Sequence
  Based on Homo sapiens sequence
PRKGGRRRRAWGKRRRRKKKRRRRRRRPRKRLRLR

SEQ ID NO: 160 - protein, 35 aa, Artificial Sequence
  Based on Homo sapiens sequence
PRKGGRRRRAWGKRRRRKKRRRRRRRRPRKRLRLR

SEQ ID NO: 161 - protein, 35 aa, Artificial Sequence
  Based on Homo sapiens sequence
PRKGGRRRRAWGKRRRRKRHRRRRRRRPRKRLRLR

SEQ ID NO: 162 - protein, 35 aa, Artificial Sequence
  Based on Homo sapiens sequence
PRKGGRRRRAWGKRRRRKRKRRRRRRRPRKRLRLR

SEQ ID NO: 163 - protein, 35 aa, Artificial Sequence
  Based on Homo sapiens sequence
PRKGGRRRRAWGKRRRRKRRRRRRRRRPRKRLRLR

SEQ ID NO: 164 - protein, 35 aa, Artificial Sequence
  Based on Homo sapiens sequence
PRKGGRRRRAWGKRRRRRHHRRRRRRRPRKRLRLR

SEQ ID NO: 165 - protein, 35 aa, Artificial Sequence
  Based on Homo sapiens sequence
PRKGGRRRRAWGKRRRRRHKRRRRRRRPRKRLRLR

SEQ ID NO: 166 - protein, 35 aa, Artificial Sequence
  Based on Homo sapiens sequence
PRKGGRRRRAWGKRRRRRHRRRRRRRRPRKRLRLR

SEQ ID NO: 167 - protein, 35 aa, Artificial Sequence
  Based on Homo sapiens sequence
PRKGGRRRRAWGKRRRRRKHRRRRRRRPRKRLRLR

SEQ ID NO: 168 - protein, 35 aa, Artificial Sequence
  Based on Homo sapiens sequence
PRKGGRRRRAWGKRRRRRKKRRRRRRRPRKRLRLR

SEQ ID NO: 169 - protein, 35 aa, Artificial Sequence
  Based on Homo sapiens sequence
PRKGGRRRRAWGKRRRRRKRRRRRRRRPRKRLRLR

SEQ ID NO: 170 - protein, 35 aa, Artificial Sequence
  Based on Homo sapiens sequence
PRKGGRRRRAWGKRRRRRRHRRRRRRRPRKRLRLR

SEQ ID NO: 171 - protein, 35 aa, Artificial Sequence
  Based on Homo sapiens sequence
PRKGGRRRRAWGKRRRRRRKRRRRRRRPRKRLRLR

SEQ ID NO: 172 - protein, 35 aa, Artificial Sequence
  Based on Homo sapiens sequence
PRKGGRRRRAWGKRRRRRRRRRRRRRRPRKRLRLR

SEQ ID NO: 173 - protein, 35 aa, Artificial Sequence
  Based on Homo sapiens sequence
PRKGGRRRRAWGRRRHRHHHRRRRRRRPRKRLRLR

SEQ ID NO: 174 - protein, 35 aa, Artificial Sequence
  Based on Homo sapiens sequence
PRKGGRRRRAWGRRRHRHHKRRRRRRRPRKRLRLR

SEQ ID NO: 175 - protein, 35 aa, Artificial Sequence
  Based on Homo sapiens sequence
PRKGGRRRRAWGRRRHRHHRRRRRRRRPRKRLRLR

SEQ ID NO: 176 - protein, 35 aa, Artificial Sequence
  Based on Homo sapiens sequence
PRKGGRRRRAWGRRRHRHKHRRRRRRRPRKRLRLR

SEQ ID NO: 177 - protein, 35 aa, Artificial Sequence
  Based on Homo sapiens sequence
PRKGGRRRRAWGRRRHRHKRRRRRRRRPRKRLRLR

SEQ ID NO: 178 - protein, 35 aa, Artificial Sequence
  Based on Homo sapiens sequence
PRKGGRRRRAWGRRRHRHRHRRRRRRRPRKRLRLR

SEQ ID NO: 179 - protein, 35 aa, Artificial Sequence
  Based on Homo sapiens sequence
PRKGGRRRRAWGRRRHRHRKRRRRRRRPRKRLRLR

SEQ ID NO: 180 - protein, 35 aa, Artificial Sequence
  Based on Homo sapiens sequence
PRKGGRRRRAWGRRRHRHRRRRRRRRRPRKRLRLR

SEQ ID NO: 181 - protein, 35 aa, Artificial Sequence
  Based on Homo sapiens sequence
PRKGGRRRRAWGRRRHRKHHRRRRRRRPRKRLRLR

SEQ ID NO: 182 - protein, 35 aa, Artificial Sequence
  Based on Homo sapiens sequence
PRKGGRRRRAWGRRRHRKHKRRRRRRRPRKRLRLR

SEQ ID NO: 183 - protein, 35 aa, Artificial Sequence
  Based on Homo sapiens sequence
PRKGGRRRRAWGRRRHRKHRRRRRRRRPRKRLRLR

SEQ ID NO: 184 - protein, 35 aa, Artificial Sequence
  Based on Homo sapiens sequence
PRKGGRRRRAWGRRRHRKKHRRRRRRRPRKRLRLR

SEQ ID NO: 185 - protein, 35 aa, Artificial Sequence
  Based on Homo sapiens sequence
PRKGGRRRRAWGRRRHRKKKRRRRRRRPRKRLRLR

SEQ ID NO: 186 - protein, 35 aa, Artificial Sequence
  Based on Homo sapiens sequence
PRKGGRRRRAWGRRRHRKKRRRRRRRRPRKRLRLR

SEQ ID NO: 187 - protein, 35 aa, Artificial Sequence
  Based on Homo sapiens sequence
PRKGGRRRRAWGRRRHRKRHRRRRRRRPRKRLRLR

SEQ ID NO: 188 - protein, 35 aa, Artificial Sequence
  Based on Homo sapiens sequence
PRKGGRRRRAWGRRRHRKRKRRRRRRRPRKRLRLR

SEQ ID NO: 189 - protein, 35 aa, Artificial Sequence
  Based on Homo sapiens sequence
PRKGGRRRRAWGRRRHRKRRRRRRRRRPRKRLRLR

SEQ ID NO: 190 - protein, 35 aa, Artificial Sequence
  Based on Homo sapiens sequence
PRKGGRRRRAWGRRRHRRHKRRRRRRRPRKRLRLR

SEQ ID NO: 191 - protein, 35 aa, Artificial Sequence
  Based on Homo sapiens sequence
PRKGGRRRRAWGRRRHRRHRRRRRRRRPRKRLRLR

SEQ ID NO: 192 - protein, 35 aa, Artificial Sequence
  Based on Homo sapiens sequence
PRKGGRRRRAWGRRRHRRKHRRRRRRRPRKRLRLR

SEQ ID NO: 193 - protein, 35 aa, Artificial Sequence
  Based on Homo sapiens sequence
PRKGGRRRRAWGRRRHRRKKRRRRRRRPRKRLRLR

SEQ ID NO: 194 - protein, 35 aa, Artificial Sequence
  Based on Homo sapiens sequence
PRKGGRRRRAWGRRRHRRKRRRRRRRRPRKRLRLR

SEQ ID NO: 195 - protein, 35 aa, Artificial Sequence
  Based on Homo sapiens sequence
PRKGGRRRRAWGRRRHRRRHRRRRRRRPRKRLRLR

SEQ ID NO: 196 - protein, 35 aa, Artificial Sequence
  Based on Homo sapiens sequence
PRKGGRRRRAWGRRRHRRRKRRRRRRRPRKRLRLR

SEQ ID NO: 197 - protein, 35 aa, Artificial Sequence
  Based on Homo sapiens sequence
PRKGGRRRRAWGRRRHRRRRRRRRRRRPRKRLRLR

SEQ ID NO: 198 - protein, 35 aa, Artificial Sequence
  Based on Homo sapiens sequence
PRKGGRRRRAWGRRRKRHHHRRRRRRRPRKRLRLR

SEQ ID NO: 199 - protein, 35 aa, Artificial Sequence
  Based on Homo sapiens sequence
PRKGGRRRRAWGRRRKRHHKRRRRRRRPRKRLRLR

SEQ ID NO: 200 - protein, 35 aa, Artificial Sequence
  Based on Homo sapiens sequence
PRKGGRRRRAWGRRRKRHHRRRRRRRRPRKRLRLR

SEQ ID NO: 201 - protein, 35 aa, Artificial Sequence
  Based on Homo sapiens sequence
PRKGGRRRRAWGRRRKRHKHRRRRRRRPRKRLRLR

SEQ ID NO: 202 - protein, 35 aa, Artificial Sequence
  Based on Homo sapiens sequence
PRKGGRRRRAWGRRRKRHKKRRRRRRRPRKRLRLR

SEQ ID NO: 203 - protein, 35 aa, Artificial Sequence
  Based on Homo sapiens sequence
PRKGGRRRRAWGRRRKRHKRRRRRRRRPRKRLRLR

SEQ ID NO: 204 - protein, 35 aa, Artificial Sequence
  Based on Homo sapiens sequence
PRKGGRRRRAWGRRRKRHRHRRRRRRRPRKRLRLR

SEQ ID NO: 205 - protein, 35 aa, Artificial Sequence
  Based on Homo sapiens sequence
PRKGGRRRRAWGRRRKRHRKRRRRRRRPRKRLRLR

SEQ ID NO: 206 - protein, 35 aa, Artificial Sequence
  Based on Homo sapiens sequence
PRKGGRRRRAWGRRRKRHRRRRRRRRRPRKRLRLR

SEQ ID NO: 207 - protein, 35 aa, Artificial Sequence
  Based on Homo sapiens sequence
PRKGGRRRRAWGRRRKRKHHRRRRRRRPRKRLRLR

SEQ ID NO: 208 - protein, 35 aa, Artificial Sequence
  Based on Homo sapiens sequence
PRKGGRRRRAWGRRRKRKHKRRRRRRRPRKRLRLR

SEQ ID NO: 209 - protein, 35 aa, Artificial Sequence
  Based on Homo sapiens sequence
PRKGGRRRRAWGRRRKRKHRRRRRRRRPRKRLRLR

SEQ ID NO: 210 - protein, 35 aa, Artificial Sequence
  Based on Homo sapiens sequence
PRKGGRRRRAWGRRRKRKKHRRRRRRRPRKRLRLR

SEQ ID NO: 211 - protein, 35 aa, Artificial Sequence
  Based on Homo sapiens sequence
PRKGGRRRRAWGRRRKRKKKRRRRRRRPRKRLRLR

SEQ ID NO: 212 - protein, 35 aa, Artificial Sequence
  Based on Homo sapiens sequence
PRKGGRRRRAWGRRRKRKKRRRRRRRRPRKRLRLR

SEQ ID NO: 213 - protein, 35 aa, Artificial Sequence
  Based on Homo sapiens sequence
PRKGGRRRRAWGRRRKRKRHRRRRRRRPRKRLRLR

SEQ ID NO: 214 - protein, 35 aa, Artificial Sequence
  Based on Homo sapiens sequence
PRKGGRRRRAWGRRRKRKRKRRRRRRRPRKRLRLR

SEQ ID NO: 215 - protein, 35 aa, Artificial Sequence
  Based on Homo sapiens sequence
PRKGGRRRRAWGRRRKRKRRRRRRRRRPRKRLRLR

SEQ ID NO: 216 - protein, 35 aa, Artificial Sequence
  Based on Homo sapiens sequence
PRKGGRRRRAWGRRRKRRHRRRRRRRRPRKRLRLR

SEQ ID NO: 217 - protein, 35 aa, Artificial Sequence
  Based on Homo sapiens sequence
PRKGGRRRRAWGRRRKRRKHRRRRRRRPRKRLRLR

SEQ ID NO: 218 - protein, 35 aa, Artificial Sequence
  Based on Homo sapiens sequence
PRKGGRRRRAWGRRRKRRKRRRRRRRRPRKRLRLR

SEQ ID NO: 219 - protein, 35 aa, Artificial Sequence
  Based on Homo sapiens sequence
PRKGGRRRRAWGRRRKRRRHRRRRRRRPRKRLRLR

SEQ ID NO: 220 - protein, 35 aa, Artificial Sequence
  Based on Homo sapiens sequence
PRKGGRRRRAWGRRRKRRRKRRRRRRRPRKRLRLR

SEQ ID NO: 221 - protein, 35 aa, Artificial Sequence
  Based on Homo sapiens sequence
PRKGGRRRRAWGRRRKRRRRRRRRRRRPRKRLRLR

SEQ ID NO: 222 - protein, 35 aa, Artificial Sequence
  Based on Homo sapiens sequence
PRKGGRRRRAWGRRRRRHHHRRRRRRRPRKRLRLR

SEQ ID NO: 223 - protein, 35 aa, Artificial Sequence
  Based on Homo sapiens sequence
PRKGGRRRRAWGRRRRRHHKRRRRRRRPRKRLRLR

SEQ ID NO: 224 - protein, 35 aa, Artificial Sequence
  Based on Homo sapiens sequence
PRKGGRRRRAWGRRRRRHHRRRRRRRRPRKRLRLR

SEQ ID NO: 225 - protein, 35 aa, Artificial Sequence
  Based on Homo sapiens sequence
PRKGGRRRRAWGRRRRRHKHRRRRRRRPRKRLRLR

SEQ ID NO: 226 - protein, 35 aa, Artificial Sequence
  Based on Homo sapiens sequence
PRKGGRRRRAWGRRRRRHKKRRRRRRRPRKRLRLR

SEQ ID NO: 227 - protein, 35 aa, Artificial Sequence
  Based on Homo sapiens sequence
PRKGGRRRRAWGRRRRRHKRRRRRRRRPRKRLRLR

SEQ ID NO: 228 - protein, 35 aa, Artificial Sequence
  Based on Homo sapiens sequence
PRKGGRRRRAWGRRRRRHRHRRRRRRRPRKRLRLR

SEQ ID NO: 229 - protein, 35 aa, Artificial Sequence
  Based on Homo sapiens sequence
PRKGGRRRRAWGRRRRRHRKRRRRRRRPRKRLRLR

SEQ ID NO: 230 - protein, 35 aa, Artificial Sequence
  Based on Homo sapiens sequence
PRKGGRRRRAWGRRRRRHRRRRRRRRRPRKRLRLR

SEQ ID NO: 231 - protein, 35 aa, Artificial Sequence
  Based on Homo sapiens sequence
PRKGGRRRRAWGRRRRRKHHRRRRRRRPRKRLRLR

SEQ ID NO: 232 - protein, 35 aa, Artificial Sequence
  Based on Homo sapiens sequence
PRKGGRRRRAWGRRRRRKHKRRRRRRRPRKRLRLR

SEQ ID NO: 233 - protein, 35 aa, Artificial Sequence
  Based on Homo sapiens sequence
PRKGGRRRRAWGRRRRRKHRRRRRRRRPRKRLRLR

SEQ ID NO: 234 - protein, 35 aa, Artificial Sequence
  Based on Homo sapiens sequence
PRKGGRRRRAWGRRRRRKKHRRRRRRRPRKRLRLR

SEQ ID NO: 235 - protein, 35 aa, Artificial Sequence
  Based on Homo sapiens sequence
PRKGGRRRRAWGRRRRRKKKRRRRRRRPRKRLRLR

SEQ ID NO: 236 - protein, 35 aa, Artificial Sequence
  Based on Homo sapiens sequence
PRKGGRRRRAWGRRRRRKKRRRRRRRRPRKRLRLR

SEQ ID NO: 237 - protein, 35 aa, Artificial Sequence
  Based on Homo sapiens sequence
PRKGGRRRRAWGRRRRRKRHRRRRRRRPRKRLRLR

SEQ ID NO: 238 - protein, 35 aa, Artificial Sequence
  Based on Homo sapiens sequence
PRKGGRRRRAWGRRRRRKRKRRRRRRRPRKRLRLR

SEQ ID NO: 239 - protein, 35 aa, Artificial Sequence
  Based on Homo sapiens sequence
PRKGGRRRRAWGRRRRRKRRRRRRRRRPRKRLRLR

SEQ ID NO: 240 - protein, 35 aa, Artificial Sequence
  Based on Homo sapiens sequence
PRKGGRRRRAWGRRRRRRHHRRRRRRRPRKRLRLR

SEQ ID NO: 241 - protein, 35 aa, Artificial Sequence
  Based on Homo sapiens sequence
PRKGGRRRRAWGRRRRRRHRRRRRRRRPRKRLRLR

SEQ ID NO: 242 - protein, 35 aa, Artificial Sequence
  Based on Homo sapiens sequence
PRKGGRRRRAWGRRRRRRKHRRRRRRRPRKRLRLR

SEQ ID NO: 243 - protein, 35 aa, Artificial Sequence
  Based on Homo sapiens sequence
PRKGGRRRRAWGRRRRRRKKRRRRRRRPRKRLRLR

SEQ ID NO: 244 - protein, 35 aa, Artificial Sequence
  Based on Homo sapiens sequence
PRKGGRRRRAWGRRRRRRKRRRRRRRRPRKRLRLR

SEQ ID NO: 245 - protein, 35 aa, Artificial Sequence
  Based on Homo sapiens sequence
PRKGGRRRRAWGRRRRRRRHRRRRRRRPRKRLRLR

SEQ ID NO: 246 - protein, 93 aa, Homo sapiens
PRKGGSRRNAWGNQSYAELISQAIESAPEKRLTLAQIYEWMVRTVPYFKDKGDSNSSAGWKNSIRHNLSLHSKFIKVHNEATGKSSWWMLNPE

SEQ ID NO: 247 - protein, 38 aa, Homo sapiens
QDLDLDMYMENLECDMDNIISDLMDEGEGLDFNFEPDP

(19) United States
(12) Patent Application Publication
(10) Pub. No.: US 2020/0255489 A1
(43) Pub. Date: Aug. 13, 2020

(54) Novel Senolytic Peptides

(71) Applicant: Eternans Ltd, Surrey (GB)

(72) Inventors: Emel Timucin, Istanbul (TR); Ugur Sezerman, Istanbul (TR); Gunseli Bayram Akcapinar, Istanbul (TR); Yavuz Ahiska, Esher (GB); Süleyman Selim Çinarouglu, Istanbul (TR)

(21) Appl. No.: 16/652,815

(22) PCT Filed: Oct. 2, 2018
(86) PCT No.: PCT/GB2018/052812

§ 371 Date: Apr. 1, 2020

Related U.S. Application Data
(60) Provisional application No. 62/567,046, filed on Oct. 2, 2017.
(60) Provisional application No. 62/567,076, filed on Oct. 2, 2017.
(60) Provisional application No. 62/567,617, filed on Oct. 3, 2017.
(60) Provisional application No. 62/622,819, filed on Jan. 26, 2018.
(60) Provisional application No. 62/712,031, filed on Jul. 30, 2018.

(51) Int. Cl. C07K 14/47

(52) U.S. Cl. CPC C07K 14/4702; A61K 38/00; C07K 2317/73

(57) ABSTRACT

Artificial senolytic peptides which selectively kill senescent cells in a mammal when administered intermittently, a process which stimulates rejuvenation in a safe manner Also, methods of use for said artificial senolytic peptides for treating senescence-associated diseases and disorders by selectively killing senescent cells. Killing senescent cells reduces the inflammatory senescence-associated secretory phenotype and therefore reduce significantly the chronic inflammation in the metabolism. The diseases and disorders treatable with said senolytic peptide include all diseases with inflammatory origin including but not restricted to diabetes, cardiovascular diseases, pulmonary diseases, including COPD; asthma, emphysema, breathlessness; renal or hepatic insufficiency, cirrhosis, osteoarthritis; senescence-associated ophthalmic diseases and disorders; and senescence-associated dermatological diseases and disorders diabetic ulcers; kyphosis; scoliosis; weight loss; hair loss; muscle loss; loss of bone density; frailty and/or reduced fitness; hearing loss such as deafness.

### CROSS-REFERENCE TO RELATED APPLICATIONS

This application is a filing under 35 U.S.C. 371 of International Application No. PCT/GB2018/052812 filed Oct. 2, 2018, which is a continuation of and claims priority to U.S. Application Ser. No. 62/567,046, filed Oct. 2, 2017 and entitled “A NOVEL EFFICACIOUS SENOLYTIC AGENT,” and to U.S. Application Ser. No. 62/567,076, filed Oct. 2, 2017 and entitled “METHOD FOR USING A NOVEL SENOLYTIC PEPTIDE FOR TREATMENT OF SENESCENCE RELATED DISEASES AND DISORDERS,” U.S. Application Ser. No. 62/567,617, filed Oct. 3, 2017 and entitled “METHOD FOR USING A NOVEL SENOLYTIC PEPTIDE FOR TREATMENT OF SENESCENCE-RELATED DISEASES AND DISORDERS,” and to U.S. Application Ser. No. 62/622,819 filed Jan. 26, 2018 and entitled “ARTIFICIAL AND EFFICACIOUS SENOLYTIC AGENT,” and to U.S. Application Ser. No. US 62/712,031 filed Jul. 30, 2018 and entitled “REPURPOSING CELL PENETRATING PEPTIDES AND THEIR NOVEL DERIVATIVES AND IOPROMIDE AND IODO-ARYL CARBONATES FOR TREATMENT OF SENESCENCE-RELATED DISEASES AND DISORDERS,” each of which is incorporated herein in its entirety.

### REFERENCE TO SEQUENCE LISTING

The content of the ASCII text file of the sequence listing named “5442.15 Sequences File-247_ST25.txt” which is 104 KB in size and was created on Sep. 30, 2018 and included herewith is incorporated herein by reference in its entirety.

### BACKGROUND

Senescence and apoptosis are among mechanisms that, when activated, restrict tumor growth. Through apoptosis, damaged cells are cleared from the organism while senescent cells remain alive in the organism though permanently restricted from entering the cell cycle. Senescence may be associated with an increase in metabolic activity. In the majority of cases, senescent cells develop a defined, but heterogeneous, secretory profile termed as senescence-associated secretory phenotype (SASP). The SASP entails release of pro-inflammatory cytokines and chemokines, tissue-damaging proteases, factors that can affect stem and progenitor cell function, haemostatic factors, and growth factors, among others. Senescent cells that express the SASP can have substantial local and systemic pathogenic effects.

The SASP secretion comprises a range of different proteins, including several proteins known to play a role in aging and age-related diseases, including matrix metalloproteases such as MMP3, growth factors, chemokines such as CCL2 and CLL11, and prominent interleukins (ILs) such as ILL IL6, and IL8. Above a certain threshold, such factors can significantly impair tissue function. The chronic SASP secretion by senescent cells may impair the functioning of neighboring cells. As such, senescent cells are thought to be major contributors of inflammation. Theoretically, low, but chronic, levels of inflammation are drivers of age-related decline in function. Consistent with this theory, senescence and SASP are elevated in a number of fast-aging mouse models and, where tested, senescence clearance delays their decline in health.

Additionally, mutations occurring in the senescent cells may lead to changes that allow those senescent cells to escape from cell cycle arrest and eventually be convert to tumorigenic cells. Thus senescent cells that display resistance to apoptosis and accumulate with age are targets of anti-aging research. The main aim of such research has been to discover the molecular pathways that direct cells to senescence but not apoptosis and, eventfully, to develop agents that interfere with these pathways so that administration of such agents will induce apoptosis in a senescent cell in a safe and predictable manner

The forkhead box (FOX) protein family comprises the FOX class O subfamily (FoxO) that has 4 mammalian members: Forkhead box protein O1 (FoxO1) (FKHR, FoxO1A), Forkhead box protein O3 (FoxO3) (FKHRL1, FoxO3A), Forkhead box protein O4 (FoxO4) (AFX, AFX1, MLLT7), and Forkhead box protein O6 (FoxO6). FoxOs are transcription factors that play important roles in suppression of tumors. Despite the fact that the exact roles of FoxOs in senescence remain to be unraveled, it has been well-established for the FoxO4 that its mRNA and protein levels have been specifically increased in response to genotoxic activation of senescence. In line with this observation, FoxO4 function was essential for senescence activation by genotoxic damage, while the loss of FoxO4 function was associated with the apoptosis of senescent cells. Hence FoxO4 has a pivotal role in the cells' direction to either senescence or apoptosis. Interfering with this key molecule FoxO4 represents efficient way of blocking senescence and steering the cells' fate towards apoptosis.

Senescence has been characterized to be a complex phenomenon that can be activated by distinct signals. In particular, genotoxic activation of senescence has been characterized by the formation of DNA-SCARS (DNA Segments with Chromatin Alterations Reinforcing Senescence). When genotoxic damage is present, FoxO4 is recruited to DNA-SCARS that also includes tumor protein 53 (p53) as a major component. Oncogenic BRAF mutation at V600E are encountered in ˜7% of all human tumors with particularly enhanced occurrence in melanoma (˜70%). Additionally, melanoma cells were found to generally have an elevated number of DNA-SCARS containing FOXO4. It has been shown that the BRAF mutation promotes the FoxO4 leading to senescence, while at the same time it also causes phosphorylation of Serine 46 (S46) of p53, the condition which was known to strongly favor apoptosis over senescence. It has been shown for the DNA-damaged melanoma cells that interference with their FoxO4 expression results in a marked increase of apoptosis. Overall these findings pointed out the FoxO4 presence inhibited the apoptosis by the S46 phosphorylated p53. Furthermore, it has been shown that inhibition of the kinase that phosphorylates the S46 of p53 led to impaired apoptosis of the senescent cells even in the absence of FoxO4. These particular observations proposed a pivotal role for FoxO4 in restraining the apoptosis mediated by the S46 phosphorylated p53. Therefore, eradicating the senescence and inducing the apoptosis via blocking the action of FoxO4 on p53 is of interest, as discussed herein.

FoxO4 inhibition for senolytic therapy using naturally occurring peptide sequences interferes with the transcriptional activity of p53 and thus lead to unwanted side effects including tumor growth. Additionally, peptide sequencesentirely derived from the human FoxO4 protein, will maintain very similar characteristics to the endogenous FoxO4 such as DNA binding and thus these peptides will also interfere with the function of FoxOs.

It is well established that chronic inflammation is the cause of many diseases. As stated above, SASP may contribute to chronic inflammation in old age. However, there is no generic senolytic agent, but a number of drugs with separate Senescent Cell Anti-apoptotic Pathways (SCAPs) and cell types some of which are Dasatinib (which acts on Dependence receptor/Src kinase/tyrosine kinase and target Primary human and mouse preadipocytes (adipose-derived stem cells)), Quercetin (which acts on Bcl-2 family, p53/p21/serpine, & PI3K/AKT and target HUVECs, mouse bone marrow-derived mesenchymal stem cells), Navitoclax (which ascts on ABT263 and target MR-90 Cells, HUVECs), Piperlongumin (which acts on A1331852/A1155463 and target IMR-90 Cells, HUVECs), and Fisetin (which acts on PI3K/AKT and targets HUVECs). The molecular pathway of the Senolytic Peptides disclosed herein are different than those mentioned in this paragraph therefore the methods of use of these Senolytic Peptide(s) for said diseases and disorders are different.

### SUMMARY OF THE INVENTION

Disclosed herein are rationally designed peptides, referred to as the “Senolytic Peptides,” including those peptides included as a part of this application in the SEQUENCE LISTING as SEQ ID NO: 1 through SEQ ID NO: 245 which are effective to induce apoptosis of senescent cells in a subject, such as a mammal, by inhibiting the action of FoxO4 on p53. In some embodiments, the Senolytic Peptides disclosed herein are unnatural peptide(s) that are optimized to maximize their interference with the FoxO4, which is up-regulated in senescent cells. Additionally or alternatively, in some embodiments, the Senolytic Peptides may be designed to effectively block the CR3 domain of FoxO4 from interfering with the DNA binding function of p53 that is phosphorylated at Serine 46, particularly, from interfering with the bulky FH domain of FoxO4. Additionally or alternatively, in some embodiments the Senolytic Peptides may be rationally designed to minimize their interaction with the DBD of p53, other FoxOs, and the DNA duplex containing a FoxO consensus binding. Compared to the prior art, the Senolytic Peptides present a safer inhibitor of FoxO4 with minimal side effects resultant from interferences from p53DBD, other FoxOs and DNA.

In some embodiments, a method for selectively inducing apoptosis of senescent cells and/or for treating a senescence-associated disease or disorder comprises administering one or more of the Senolytic Peptides which minimize the interaction between FoxO4 and p53. In various embodiments, the method may comprise administering one or more of the Senolytic Peptide(s) via a treatment regime as disclosed herein or some variation thereof. In various embodiments, treatment regimes may vary as to administration frequency and/or dosing (for example, as to the a dosage that will be therapeutically effective). Particular treatment regimes may be specifically developed to treat different types of senescence-associated diseases or disorders. Induction of apoptosis in senescent cells (that is, killing senescent cells) reduces the inflammatory senescence-associated secretory phenotype and therefore significantly reduce the chronic inflammation in the metabolism. Further, said treatment may stimulate overall rejuvenation in a safe manner Thus, the diseases and disorders treatable via the Senolytic Peptides may include, but not limited to, all diseases with inflammatory origin including diabetes, cardiovascular diseases, pulmonary diseases, osteoarthritis; senescence-associated ophthalmic diseases and disorders; and senescence-associated dermatological diseases and disorders. The applicable regimes and individualization of the treatment for said diseases are presented under corresponding headings within this application.

In some embodiments, the Senolytic Peptides may not have to be continuously present to exert an effect. For examples, brief disruption of pro-survival pathways, such as by administration of a Senolytic Peptide, is adequate to kill senescent cells. Thus, in some embodiments, the Senolytic Peptides are suitable to be administered intermittently.

It is to be understood that both the foregoing general description and the following detailed description are exemplary and explanatory only and are not restrictive of the invention, as claimed.

### BRIEF DESCRIPTION OF THE DRAWINGS

FIG. 1A illustrates homology models of the FH of FoxO4 as predicted by SWISS-MODEL.

FIG. 1B illustrates homology models of the CR3 domains of FoxO4 as predicted by SWISS-MODEL.

FIG. 2 illustrates the complex formed by the FH and the CR3 domains of FoxO4 that was predicted by HADDOCK.

FIG. 3A illustrates the complex formed by the p53DBD and the FH domain of FoxO4 as predicted by HADDOCK.

FIG. 3B illustrates the complex formed by the p53DBD and the CR3 domain of FoxO4 as predicted by HADDOCK.

FIG. 4A illustrates the binding interfaces of the docked complexes of p53DBD and FoxO4. For comparison, p53DBD shown in FIGS. 4A and 4B were superimposed.

FIG. 4B illustrates the complex structure of p53DBD and its consensus DNA (PDB ID: 3kmd). The complex structures of p53, shown in FIGS. 4A and 4B confirms that the CR3 domain of FoxO4 and DNA binds to the same surface in the p53DBD. For comparison, p53DBD shown in FIGS. 4A and 4B were superimposed.

FIG. 5A illustrates the complex formed by the FH domain of FoxO4 with the CR3 domain of FoxO1 which was predicted by HADDOCK. Both of the FH domain of FoxO4 (FIG. 2) and the CR3 domain of FoxO1 were modeled using SWISS-MODEL. For comparison, FH domains of the complexes shown here and in FIG. 2 were superimposed

FIG. 5B illustrates the complex formed by the FH domain of FoxO4 with the CR3 domain of FoxO3 (FIG. 2) which was predicted by HADDOCK. The FH domain of FoxO4 was obtained by SWISS-MODEL while the CR2C-CR3 domain of FoxO3 were obtained from the crystal structure (PFB ID: 21qh).For comparison, FH domains of the complexes shown here and in FIG. 2 were superimposed.

FIG. 6A illustrates the complex formed by the FH domain of FoxO4 and its consensus DNA which was obtained from the crystal structure (PDB ID: 312c). The FH domains shown here and in FIG. 6B were superimposed for comparision. The CR3 domain of FoxO4 and FoxO consensus DNA binds to the same surface in the FH domain of FoxO4.

FIG. 6B illustrates the complex formed by the interface FH 602 and CR3 601 domains of the FoxO4 (the same complex shown in FIG. 2) which was predicted by HADDOCK. The FH domains shown here and in FIG. 6B were superimposed for comparision. The CR3 domain of FoxO4 and FoxO consensus DNA binds to the same surface in the FH domain of FoxO4.

FIG. 7A illustrates the critical findings of the NMR study (Wang et al., 2008) revealing the intramolecular and intermolecular interactions of the CR3 domain of FoxO4 with the FH domain of FoxO4 and p53 respectively. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53.

FIG. 7B illustrates the rationale of the disclosed subject matter. (A) T. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53. Given the findings of the NMR study which was illustrated in FIG. 7A, the rationale behind switching on the p53 mediated apoptosis relies on the saturation (inhibition) of the CR3 of FoxO4 by the designed peptides that possess a similar binding surface with the FH domain.

FIG. 8A illustrates possible interaction partners of the FH domain of FoxO4. FH domain of FoxO4 can interact with FoxO consensus DNA. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53. The peptides were optimized against the multi-targets shown in FIGS. 8A-D, such that the peptides that increase the affinity toward the CR3 domain of FoxO4 (FIG. 8D) but reduce it to CR3 domains of FoxO4 (FIG. 8B), to consensus FoxO4 DNA (FIG. 8A) and p53DBD (FIG. 8C). The specificity of the designed peptides was maximized towards FoxO4 that was marked with a shadow of the peptide representation, while the specificity towards any other interaction partners was kept at minimal level that is represented by cross signs.

FIG. 8B illustrates possible interaction partners of the FH domain of FoxO4. FH domain of FoxO4 can interact with CR3 domains of other FoxOs that are represented by an asterisk symbol. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53. The peptides were optimized against the multi-targets shown in FIG. 8, such that the peptides that increase the affinity toward the CR3 domain of FoxO4 (FIG. 8D) but reduce it to CR3 domains of FoxO4 (FIG. 8B), to consensus FoxO4 DNA (FIG. 8A) and p53DBD (FIG. 8C). The specificity of the designed peptides was maximized towards FoxO4 that was marked with a shadow of the peptide representation, while the specificity towards any other interaction partners was kept at minimal level that is represented by cross signs.

FIG. 8C illustrates possible interaction partners of the FH domain of FoxO4. FH domain of FoxO4 can interact with the DBD of p53. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53. The peptides were optimized against the multi-targets shown in FIG. 8, such that the peptides that increase the affinity toward the CR3 domain of FoxO4 (FIG. 8D) but reduce it to CR3 domains of FoxO4 (FIG. 8B), to consensus FoxO4 DNA (FIG. 8A) and p53DBD (FIG. 8C). The specificity of the designed peptides was maximized towards FoxO4 that was marked with a shadow of the peptide representation, while the specificity towards any other interaction partners was kept at minimal level that is represented by cross signs.

FIG. 8D illustrates possible interaction partners of the FH domain of FoxO4. FH domain of FoxO4 can interact with the CR3 domain of FoxO4. FH stands for Forkhead that is the DNA binding domain at the N-terminal of the FoxO4. CR3 stands for the Conserved Region 3 that is found at the C-terminal of FoxO4. The p53DBD indicated the DNA binding domain of p53. The peptides were optimized against the multi-targets shown in FIG. 8, such that the peptides that increase the affinity toward the CR3 domain of FoxO4 (FIG. 8D) but reduce it to CR3 domains of FoxO4 (FIG. 8B), to consensus FoxO4 DNA (FIG. 8A) and p53DBD (FIG. 8C). The specificity of the designed peptides was maximized towards FoxO4 that was marked with a shadow of the peptide representation, while the specificity towards any other interaction partners was kept at minimal level that is represented by cross signs.

FIG. 9 illustrates heat-map from ProteomicsDB database indicating the differences in expression levels of FoxO1, 3, and 4 in various tissues based on z-scored mRNA expression profiles.

FIG. 10A illustrates docking of the N-terminal of FH comprising the residues from 1 to 35 (FH-Nter¹⁻³⁵) to FoxO DNA as predicted by HADDOCK. The backbone of the FH-Nter¹⁻³⁵ shown in FIGS. 10A, B, C, D and E were superimposed.

FIG. 10B illustrates docking of the N-terminal of FH comprising the residues from 1 to 35 (FH-Nter¹⁻³⁵) to the CR2C-CR3 of FoxO1 as predicted by HADDOCK. The backbone of the FH-Nter¹⁻³⁵ shown in FIGS. 10A, B, C, D and E were superimposed.

FIG. 10C illustrates docking of the N-terminal of FH comprising the residues from 1 to 35 (FH-Nter¹⁻³⁵) to the CR3 of FoxO3 as predicted by HADDOCK. The backbone of the FH-Nter¹⁻³⁵ shown in FIGS. 10A, B, C, D and E were superimposed.

FIG. 10D illustrates docking of the N-terminal of FH comprising the residues from 1 to 35 (FH-Nter¹⁻³⁵) to p53DBD as predicted by HADDOCK. The backbone of the FH-Nter¹⁻³⁵ shown in FIGS. 10A, B, C, D and E were superimposed.

FIG. 10E illustrates docking of the N-terminal of FH comprising the residues from 1 to 35 (FH-Nter¹⁻³⁵) to the CR3 of FoxO4 as predicted by HADDOCK. The backbone of the FH-Nter¹⁻³⁵ shown in FIGS. 10A, B, C, D and E were superimposed.

FIG. 11A illustrates the positions at which the peptide(s) is mutated from the native FoxO4. The mutations present in the peptides shown in the SEQUENCE LISTING as SEQ ID NO: 1 through SEQ ID NO: 245 are represented by spheres in the backbone of the FH-Nter¹⁻³⁵ that is bound to the CR3 of FoxO4.

FIG. 11B illustrates the positions at which the peptide(s) is mutated from the native FoxO4. The mutations present in the peptides shown in the SEQUENCE LISTING as SEQ ID NO: 1 through SEQ ID NO: 245 are represented by spheres in the backbone of the FH-Nter¹⁻³⁵ that is bound to the DNA.

FIG. 11C illustrates the positions at which the peptide(s) is mutated from the native FoxO4. The mutations present in the peptides shown in the SEQUENCE LISTING as SEQ ID NO: 1 through SEQ ID NO: 245 are represented by spheres in the backbone of the FH-Nter¹⁻³⁵ that is bound to the CR3 of FoxO1.

FIG. 11D illustrates the positions at which the peptide(s) is mutated from the native FoxO4. The mutations present in the peptides shown in the SEQUENCE LISTING as SEQ ID NO: 1 through SEQ ID NO: 245 are represented by spheres in the backbone of the FH-Nter¹⁻³⁵ that is bound to the CR2C-CR3 of FoxO3.

FIG. 11E illustrates the positions at which the peptide(s) is mutated from the native FoxO4. The mutations present in the peptides shown in the SEQUENCE LISTING as SEQ ID NO: 1 through SEQ ID NO: 245 are represented by spheres in the backbone of the FH-Nter¹⁻³⁵ that is bound to p53DBD.

FIG. 12A illustrates the molecular interactions at the binding interface of the complexes formed by the FH-Nter¹⁻³⁵ and the CR3 of FoxO4.

FIG. 12B illustrates the molecular interactions at the binding interface of the complexes formed by the FH-Nter¹⁻³⁵ and FoxO DNA.

FIG. 12C illustrates the molecular interactions at the binding interface of the complexes formed by the FH-Nter¹⁻³⁵ and the CR3 of FoxO1.

FIG. 12D illustrates the molecular interactions at the binding interface of the complexes formed by the FH-Nter¹⁻³⁵ and the CR2C-CR3 of FoxO3.

FIG. 12E illustrates the molecular interactions at the binding interface of the complexes formed by the FH-Nter¹⁻³⁵ and p53DBD.

FIG. 13A illustrates the complex interactions of p53 TAD (PDB ID: 2b3g) with the FH domain of FoxO4. The S46 was mutated to aspartate (D) as the phosphate mimic

FIG. 13B illustrates the complex interactions of p53 TAD (PDB ID: 2b3g) with the FH-Nter¹⁻³⁵. The S46 was mutated to aspartate (D) as the phosphate mimic. The peptides shown in the SEQUENCE LISTING as SEQ ID NO: 1 through SEQ ID NO: 245 may be effective to attenuate these interactions to enhance the apoptotic function of the S46 phosphorylated p53.

FIG. 14A illustrates the dose in which the Senolytic Peptides shall be administered following guidance provided in the Impulse Regime. The Therapeutically Effective Dose and frequency of administration may vary within this regime. The Impulse Regime presents an optional follow up treatment course in which the Therapeutically Effective Dose may be increased or decreased. FIG. 14A depicts at least six different considerations: (1) Treatment Cycle (comprised of subject's initial assessment, administration of Therapeutically Effective Dose and final assessment); (2) Therapeutically Effective Dose delivered in 1, 2, or 3 days; (3) Optional further treatment cycle(s) may be required if senescent cell reduction is unsatisfactory; (4) Senescence Clearance Interval 2-4 weeks; (5) Initial senescence assessment via biopsy samples; and (6) Final senescence assessment via biopsy samples.

FIG. 14B illustrates the dose in which the Senolytic Peptides shall be administered following guidance provided in the Sustained Regime. The Therapeutically Effective Dose and frequency of administration may vary within this regime. The Sustained Regime presents an optional follow up treatment course in which the Therapeutically Effective Dose may be increased or decreased. FIG. 14B depicts: (1) Treatment Cycle (comprised of subject's initial assessment, administration of Therapeutically Effective Dose and final assessment); (2) Therapeutically Effective Dose delivered in 1, 2, or 3 weeks; (3) Optional further treatment cycle(s) may be required if senescent cell reduction is unsatisfactory. Therapeutically Effective Dose may be readjusted; (4) Senescence Clearance Interval 2-4 weeks; (5) Initial senescence assessment via biopsy samples; and (6) Final senescence assessment via biopsy samples.

FIG. 14C illustrates the dose in which the Senolytic Peptides shall be administered following guidance provided in the Gentle Regimes. The Therapeutically Effective Dose and frequency of administration may vary within this regime. The Gentle Regime presents an optional follow up treatment course in which the Therapeutically Effective Dose may be increased or decreased. FIG. 14C depicts: (1) Treatment Cycle (comprised of subject's initial assessment, administration of Therapeutically Effective Dose and final assessment); (2) Therapeutically Effective Dose delivered in 3 or 3 weeks; (3) Optional further treatment cycle(s) may be required if senescent cell reduction is unsatisfactory. Therapeutically Effective Dose may be readjusted; (4) Senescence Clearance Interval 2-4 weeks; (5) Initial senescence assessment via biopsy samples; and (6) Final senescence assessment via biopsy samples.

FIG. 15A illustrates SA-B-Gal positive, senescent IMR90 cells were produced by treatment with 100 NM Doxorubicin.

FIG. 15B illustrates Senolytic effect of the peptide listed as SEQ ID NO: 11 in the SEQUENCE LISTING on Doxorubicin induced senescent and non-senescent IMR90 cells.

FIG. 15C illustrates the Senolytic effect of FoxO4 DRI on Doxorubicin induced senescent and non-senescent IMR90 cells.

FIG. 16A illustrates SA-B-Gal positive, senescent WI-38 cells were produced by treatment with 100 NM Doxorubicin.

FIG. 16B illustrates Senolytic effect of the peptide listed as SEQ ID NO: 11 in the SEQUENCE LISTING on Doxorubicin induced senescent and non-senescent WI-38 cells.

FIG. 16C illustrates Senolytic effect of FoxO4 DRI on Doxorubicin induced senescent and non-senescent WI-38 cells.

FIG. 17A illustrates Experimental set up.

FIG. 17B illustrates Doxorubicin administered and the peptide listed as SEQ ID NO: 11 in the SEQUENCE LISTING treated mouse before and after Senolytic Peptide Treatment showing alopecia and reversal of alopecia, respectively.

FIG. 17C illustrates SA-B-Gal staining of Saline treated, Doxorubicin +PBS treated and Doxorubicin +the peptide listed as SEQ ID NO: 11 in the SEQUENCE LISTING treated mice kidney samples. Each kidney was flash-frozen in liquid nitrogen, immediately sectioned (10 μm) and stained via SA-(3-GAL staining. Mid-saggital sections were taken and multiple images were taken from similar depths.

FIG. 18 illustrates the position at which the peptides disclosed herein contain at least one mutation.

FIG. 19 illustrates haematological parameters measured from blood samples collected from BALB/c miced administered with SEQ DI NO: 11 at doses of 1 mg/kg, 10 mg/kg, 50 mg/kg, and 100 mg/kg of body weight according to Acute Systemic Toxicity Test (ISO 10993-=11). Statisitcally significant changes were shown (P<0.05).

FIG. 20A illustrates the Senolytic effect of the peptide (SEQ ID NO: 12) on Doxorubicin induced senescent and non-senescent WI-38 cells.

FIG. 20B illustrates the Senolytic effect of the peptide (SEQ ID NO: 12) on Doxorubicin induced senescent and non-senescent IMR90 cells.

### DETAILED DESCRIPTION OF THE INVENTION

With respect to the following disclosure, the following definitions apply:

“Administration” or “administering,” as used herein, refers to a method of giving a dosage of a compound or composition to subject, such as a vertebrate or invertebrate, including a mammal, a bird, a fish, or an amphibian, via a suitable mode of administration, for example, intrarespiratory, topical, oral, intravenous, intraperitoneal, intramuscular, buccal, rectal, sublingual. In various embodiments as will be disclosed herein, the preferred mode of administration can vary depending on various factors, such as the components being administered, the tissue site being targeted (e.g., a tissue in which the disease or disorder resides, is present, or is manifested), the particular disease or disorder involved, and the severity of the disease or disorder.

“Senescence related diseases or disorders,” as used herein, refers to, but is not limited to, any of the following diseases or disorders: renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. A prominent feature of aging is a gradual loss of function, or degeneration that occurs at the molecular, cellular, tissue, and organismal levels. Age-related degeneration gives rise to well-recognized pathologies, such as sarcopenia, atherosclerosis and heart failure, osteoporosis, pulmonary insufficiency, renal failure, neurodegeneration including macular degeneration, Alzheimer's disease, and Parkinson's disease, and many others. Although varying considerably, age-related pathologies generally become more prevalent with approximately exponential kinetics, beginning approximately 50 years of age for humans and mid-life for mammals

“Autoimmune diseases or disorders,” as used herein, refers to autoimmune diseases or disorders such, but not limited to, osteoporosis, psoriasis, oral mucositis, rheumatoid arthritis, inflammatory bowel disease, eczema, kyphosis, herniated intervertebral disc, and the pulmonary diseases, COPD, and idiopathic pulmonary fibrosis.

“Biological sample” or “biopsy sample,” as used herein, refers to a biological sample which is obtained from a subject by invasive, non-invasive or minimally invasive methods, for example, a blood sample, a serum sample, a plasma sample, a biopsy specimen, body fluids (for example, lung lavage, ascites, mucosal washings, synovial fluid, vitreous fluid, or spinal fluid), bone marrow, lymph nodes, tissue explant, skin tissue sample, vaginal tissue, organ culture, or any other tissue or cell preparation from a subject.

“Cancer,” as used herein, refers to, but is not limited to, cancers which are solid tumors or liquid tumors. Solid tumors may include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer (including squamous cell skin cancer), renal cancer, head and neck cancers, throat cancer, squamous carcinomas(e.g., that form on the moist mucosal linings of the nose, mouth, throat, etc.), bladder cancer, osteosarcoma (bone cancer), cervical cancer, endometrial cancer, esophageal cancer, liver cancer, and kidney cancer and further including the metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells. Liquid tumors may include, for example, cansers occuring in blood, bone marrow, and lymph nodes and include generally, leukemias (myeloid and lymphocytic) including acute lymphoblastic leukemia (ALL), acute myeloid leukemia (AML), chronic lymphocytic leukemia (CLL), chronic myelogenous leukemia (CML), and hairy cell leukemia lymphomas (e.g., Hodgkin lymphoma), and melanoma, including multiple myeloma).

“Cardiovascular disease,” as used herein, refers to, but is not limited to, angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance, and stroke.

“Inflammatory or autoimmune diseases or disorders,” as used herein, refers to, but is not limited to, inflammatory diseases or disorders, such as by way of non-limiting example, osteoarthritis, or autoimmune diseases or disorders, such as by way of non-limiting example, osteoporosis, psoriasis, oral mucositis, rheumatoid arthritis, inflammatory bowel disease, eczema, kyphosis, herniated intervertebral disc, and the pulmonary diseases, COPD and idiopathic pulmonary fibrosis.

“Peptidomimetics,” as used herein, refers to certain chemical compounds mimicking a natural peptide with the ability to interact with the target and exert the same biological effect. Peptidomimetics may be often implemented to overcome problems associated with the intrinsic properties of natural peptides such as stability against proteolytic degradation. Peptide bond surrogates and (3-turn dipeptide mimetics are included among examples of peptidomimetics.

“Pulmonary diseases and disorders,” as used herein, refers to, but is not limited to, idiopathic pulmonary fibrosis (IPF), chronic obstructive pulmonary disease (COPD), asthma, cystic fibrosis, bronchiectasis, and emphysema.

“Senescence-associated dermatological diseases and disorders,” as used herein, refers to, but is not limited to, psoriasis, vitiligo, and eczema, which are also inflammatory diseases and are discussed in greater detail. Other dermatological diseases and disorders that may be associated with senescence include rhytides (wrinkles due to aging); pruritis (linked to diabetes and aging); dysesthesia (chemotherapy side effect that is linked to diabetes and multiple sclerosis); psoriasis (as noted) and other papulosquamous disorders, for example, erythroderma, lichen planus, and lichenoid dermatosis; atopic dermatitis (a form of eczema and associated with inflammation); eczematous eruptions (often observed in aging subjects and linked to side effects of certain drugs). Other dermatological diseases and disorders associated with senescence include cutaneous lymphomas, eosinophilic dermatosis (linked to certain kinds of hemotologic cancers); reactive neutrophilic dermatosis; pemphigus, cutaneous lupus, pemphigoid and other immunobullous dermatosis fibrohistocytic proliferations of skin.

“Senolytics,” as used herein, refers to one or more of the Senolytic Peptides or a senolytic agents.

“Senescence-associated diseases and disorders,” as used herein, refers to, but is not limited to, a senescence-related disease or disorder, an inflammatory disease or disorder, an autoimmune disease or disorder, a cardiovascular disease or disorder, a pulmonary disease or disorder, an eye disease or disorder, a metabolic disease or disorder, a neurological disease or disorder, a senescence-associated dermatological disease or disorder, a nephrological disease or disorder.

“Senolytic Agent,” as used herein, refers to, but is not limited to: Dasatinib, Quercetin, Navitoclax, Piperlongumin, Fisetin, BCL-XL inhibitors A1331852 and A1155463, FoxO4-related peptides.

“Senolytic Peptide(s),” as used herein, refers to the novel, artificial peptide sequences disclosed herein, including the peptides shown in the SEQUENCE LISTING as SEQ ID NO: 1 through SEQ ID NO: 245, more specifically SEQ ID NO: 1 through SEQ ID NO: 12, more specifically SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or SEQ ID NO: 11, or SEQ ID NO: 12, and derivatives thereof.

“Subject,” as used herein, refers to a target of a treatment or therapy, including but not limited to, a human or a non-human mammal, a dog, a cat, a mouse, a rat, a cow, a sheep, a pig, a goat, a non-human primate or a bird, for example, a chicken, as well as any other vertebrate or invertebrate.

“Treat,” “treatment,” or “treating,” as used herein, refers to administration of a composition for therapeutic purposes.

Disclosed herein is a successful model for the interaction network between the domains of FoxO4 and p53 at atomic resolution, by which the selective inhibition of the action of FoxO4 on p53 was achieved.

Both FoxO4 and p53 have multiple domains with distinct functions. Prior art investigations as to possible interactions of FoxO4 with p53 implied that FoxO4 can interact with p53 through multiple domains. Not intending to be bound by theory, these potential interactions between FoxO4 and p53 may explain the cause of the restrain elicited by FoxO4 on the apoptotic function of p53. Hence in the rational design, the possible complexes of FoxO4 and p53 was elucidated at the atomic resolution.

The FoxO family of proteins express similar domain compositions such that they contain DNA binding domains entitled as the Forkhead (FH) domain and C-terminal domains entitled as CR1, CR2 and CR3 for transactivation. The FH and CR3 domains interact with each other and the binding surface of these interactions have been resolved in an NMR based study using FoxO3. FH includes basic (positively charged) and hydrophobic amino acids (R153, R154, W157 and G158) that contribute to this intramolecular interaction, while a central portion of CR3 that includes acidic (negatively charged) and hydrophobic amino acids (the amino acids from D623 to M633) interacts with the FH.

This NMR study also shed light on the action of FoxO3 on p53. Essentially, in vitro pull down assays were used to identify the critical domain(s) of both p53 and FoxO3 for their intermolecular interactions. According to these results, the DNA binding domain of p53 (p53DBD) was found to be necessary for binding to the FoxO3, while the C-terminal portion of the FoxO3 that includes the CR3 domain was revealed to be the most critical domain for p53 interaction. Further experiments showed that addition of the p53DBD disrupted the intramolecular interaction between the FH and the CR3 domains of FoxO3, in that the binding affinities of both of the FH-CR3 and p53DBD-CR3 complexes were comparable with each other. As resolved by NMR shift experiments, the binding interface of the CR3 domain of FoxO3 has been overlapped in both of the complexes formed by the FH domain of FoxO3 and the p53DBD. This particular finding implied a competition of the FH domain of FoxO3 with the p53DBD to bind to the same surface on the CR3 domain of FoxO3. Sequestration of the CR3 domain, particularly from the surface that it binds to p53DBD will circumvent its interaction with the p53DBD, liberating the p53DBD and its transcriptional activity to initiate apoptosis.

The FoxO proteins are very similar proteins that share high sequence similarity and domain composition. This similarity in the protein sequence was further observed at the functional level by in vivo experiments in mice. The resulting functional redundancy will suggest the validity of the findings on the FoxO3 for FoxO4. Hence, in connection to the insights obtained from the NMR findings, blocking of the CR3 domain of FoxO4 has the potential to inhibit the interaction between FoxO4 and p53 and thus liberate p53 in senescent cells.

Interference in the p53 structure or pathway may bear harmful consequences to cells and thus should be avoided by any means. Nevertheless the structural findings suggested that the CR3 domain of FoxO4 binds to and possibly inhibits the function of the p53DBD. Blocking of the CR3 domain will inhibit “the inhibition of the p53DBD” and will promote the function of p53DBD. Experiments conversely showed that promoting the inhibition of p53 by an oncoprotein, named gankryin, enhanced tumor growth. Gankyrin acts as a promoter of MDM2 which is the well-known inhibitor of p53. Thus interference with the CR3 domain of FoxO4, without any direct interference with the p53 protein, will have the potential to remove the said restrain on the p53 mediated apoptosis by FoxO4, eradicating the senescence.

In some embodiments, the FH-CR3 complex model resolved the inhibition of CR3 domain at atomic resolution. The subunits of this complex were generated using SWISS-MODEL. The structure of the full-length FH domain of FoxO4 (PRKGGSRRNAWGNQSYAELIS QAIES APEKRLTLAQIYEWMVRTVPYFKD KGDS NS S AGWKNSIRHNLSLHSKFIKVHNEATGKSSWWMLNPE, included in the SEQUENCE LISTING as SEQ ID NO: 246) was modeled using the crystal structure (PDB ID: 312c) as the template. Although the C-terminal of the FoxOs were identified to be unstructured in solution, the central core region of CR3 of FoxO4 was predicted to be in helical form by a secondary structure prediction algorithm and circular dichroism spectroscopy.

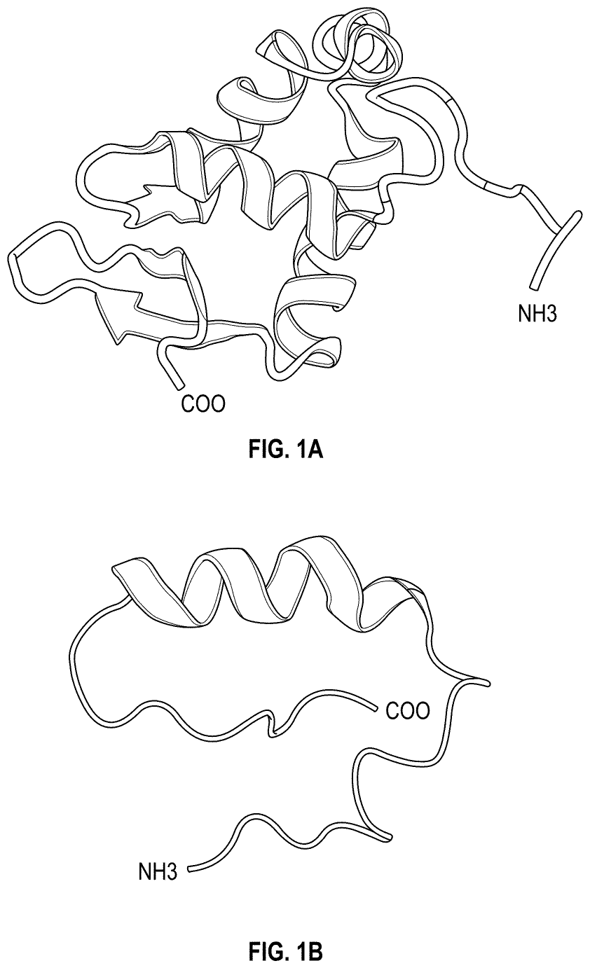

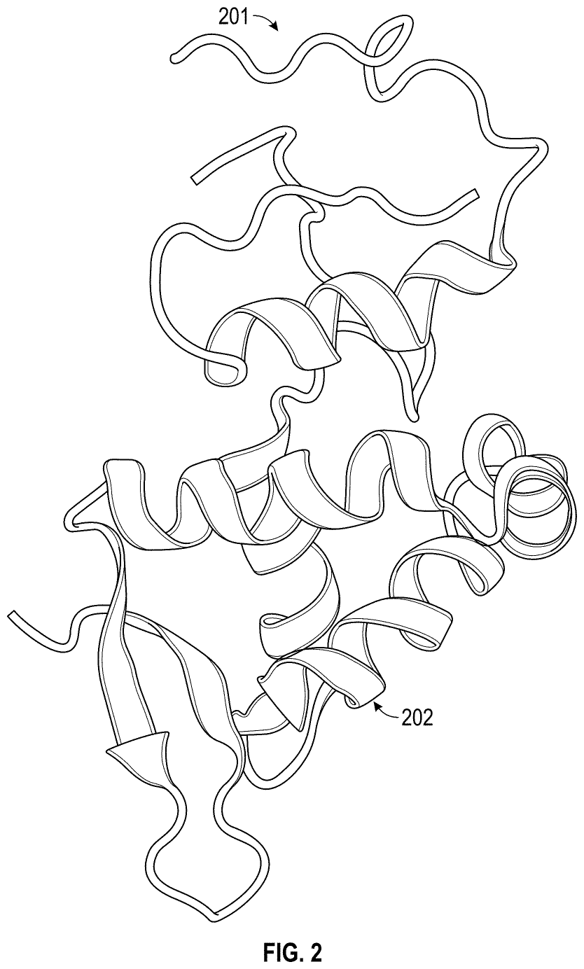

Furthermore, a crystallization study that resolved the C-terminal CR2 and CR3 (CR2C-CR3) domains of FoxO3 (PDB ID: 21qh) suggested parallel results with these predictions and displayed that the CR3 region forms a helical structure. Hence, when the modeling of the CR3 domain (QDLDLDMYMENLECDMDNIISDLMDEGEGLDFNFEPDP, included in the SEQUENCE LISTING as SEQ ID NO: 247), this structure (PDB ID: 21qh) was used as the template. These models of the FH and CR3 domains of FoxO4 were shown in FIG. 1A and 1B respectively. These models were used in the molecular docking simulations by using HADDOCK. The active residues that were included in the HADDOCK calculations were retrieved from the NMR study. The resulting complex was shown in FIG. 2, which illustrates the CR3 domain 201 and the FH domain 202.

In some embodiments, the interaction of FH domain of FoxO4 with the DBD of p53 was unraveled. The complexes of the DBD of p53 formed by

(i) the FH of FoxO4

(ii) the CR3 of FoxO4

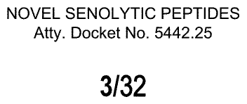

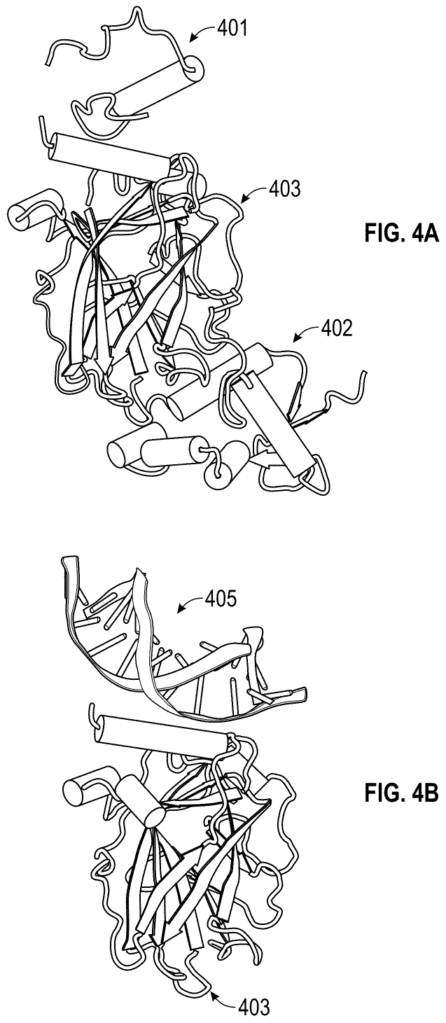

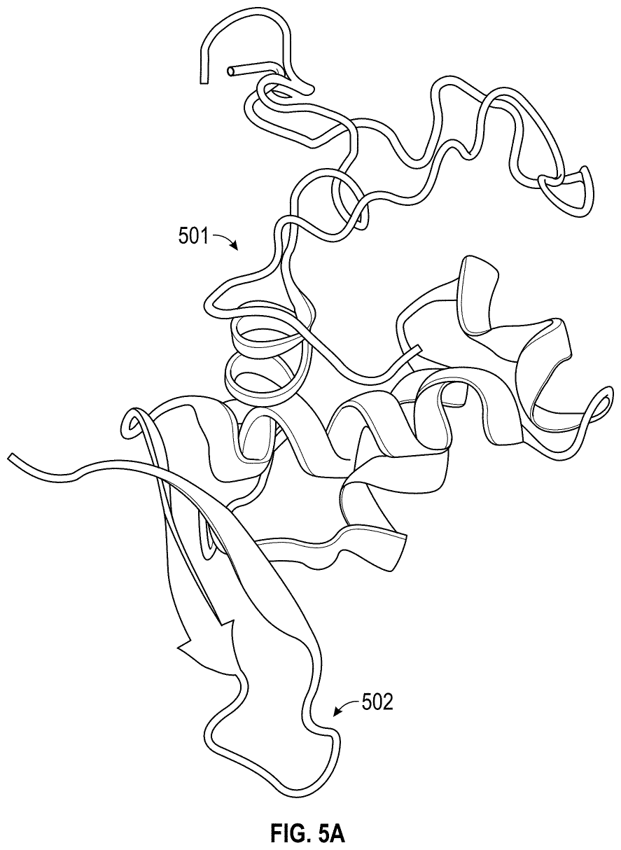

were built by the homology models of the FH (FIG. 1A), the CR3 of FoxO4 (FIG. 1B) and the crystal structure (PDB ID: 3kmd). The active residues for FH and CR3 were taken from the NMR study while the active residues for p53DBD were predicted to be the positively charged residues found on the surface. Despite the fact that the binding interface of the FH-p53DBD complex was not resolved, due to the high number of positively charged residues in the FH domain, a negatively charged interaction surface was tagged as active on the p53 for FH interaction. Explicitly, R273 was selected as the active residue of p53DBD for CR3 interaction, while it was E221 and E224 for FH interaction. The results from HADDOCK calculations were given in the FIG. 3A and 3B for the complexes FH-p53 and CR3-p53, respectively. When p53DBD 303 was superimposed in both models, it was revealed that the FH 302 and CR3 301 bind to distinct p53DBD 303 surfaces (FIGS. 3A and 3B). This observation unraveled the model for the full-length FoxO4 interaction with the p53DBD 403 (FIG. 4A). The CR3 domain 401 interacted with the DBD of p53 403 (FIG. 4A) from the same surface that was recruited for the DNA 405 interaction of p53 403 (PDB ID: 3kmd) (FIG. 4B). This particular observation indicates that the interaction of the CR3 401 of FoxO4 402with the DBD of p53 403 sequesters p53 403 from the transcriptional activity, highlighting the importance of blocking CR3 401 to liberate p53 403 activity. These structural models elucidated the molecular mechanism behind the apoptosis restrain elicited by FoxO4, highlighting the significance of blocking the CR3 domain 401 of FoxO4 in promoting the DNA binding function of p53 403.

In some embodiments, the possible interaction between FH domain of FoxO4 and the CR3 domain of other FoxO members was revealed. Due to the functional redundancy in FoxO members, inhibition of the CR3 domain of FoxO4 is circumvented by the interactions with the other FoxOs and DNA as well. Hence to enhance specificity of the design towards only FoxO4, the interactions between

(i) the FH of FoxO4 and the CR3 of another FoxO member, FoxO1

(ii) the FH of FoxO4 and the CR3 of another FoxO member, FoxO3

(iii) the FH of FoxO4 and DNA

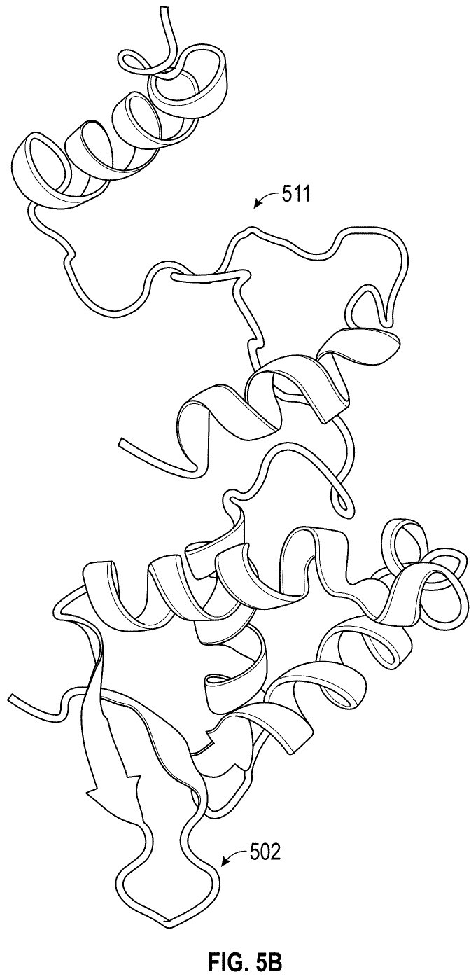

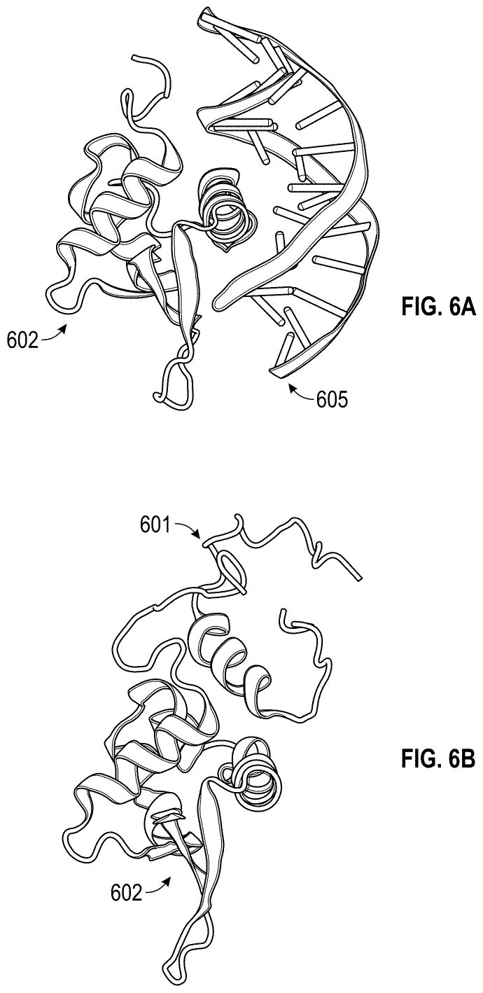

were also modeled. The first complex was generated by the HADDOCK using the homology models of the FH (FIG. 1A) and the CR3 domain 501 of FoxO1 (Figure SA) that were generated by SWISS-MODEL. The second complex was generated by the HADDOCK using the homology models of the FH (FIG. 1A) and the crystal structure of the CR3 of FoxO3 (PDB ID: 21qh). The active residues were obtained from the NMR shift experiment. The FoxO1-FoxO4 and FoxO3-FoxO4 complex formed by the FH 502 of FoxO4 and the CR3 501 of FoxO1 and FoxO3 were demonstrated in Figure SA. CR3 501 of FoxO1 (Figure SA) and CR2C-CR3 511 of FoxO3 (FIG. 5B) bind to the same surface of FH 202 with the CR3 201 of FoxO4 (FIG. 2). This similarity in the binding surfaces of different FoxO proteins implied a possible interference of other FoxO members with the inhibition of the CR3 of FoxO4. The third complex was obtained from the crystal structure (PDB ID: 312c) and shown in FIG. 6A (FH 602of FoxO4, DNA 605).

The complexes that were used are listed in Table 1.

TABLE 1
Complex structures.

| Complex | Partner 1 | | Partner 2 | | Complex |
|---|---|---|---|---|---|
| Number | Name | Structure | Name | Structure | Structure |
| 1 | FH | FoxO4 model | CR3 | FoxO4 model | Disclosed herein |
| 2 | FH | FoxO4 model | p53 DBD | PDB ID: 3kmd | Disclosed herein |
| 3 | CR3 | FoxO4 model | p53 DBD | PDB ID: 3kmd | Disclosed herein |
| 4 | FH | FoxO4 model | FoxO1 CR3 | FoxO1 model | Disclosed herein |
| 5 | FH | FoxO4 model | FoxO3 CR3 | PDB ID: 21qh | Disclosed herein |
| 6 | FH | PDB ID: 312c | FoxO DNA | PDB ID: 312c | [1] |
| 7 | FH | FoxO4 model | p53 TAD | PDB ID: 2b3g | Disclosed herein |

[1] Wang, F., et al., Biochemical and structural characterization of an intramolecular interaction in FOXO3a and its binding with p53. J Mol Biol, 2008. 384(3): p. 590-603

These models enabled structural investigations at the atomic resolution and optimization of the selectivity of the Senolytic Peptide(s). Due to the fact that the N-terminal of FH domain was found interacting in all of the complexes, the N-terminal of the FH domain was selected in the rational design of CR3 inhibition. The length was decided according to the visual inspections of the full-length FH domain structure. The N-terminal of the FH domain has a single a helix encompassing the residues between S15 and S26. The proceeding secondary structure is a (3 bridge that connects to the second a helix that is formed by the residues between A35 and V45. During design of the CR3 inhibitory peptides, the first 35 amino acids of the FH domain of FoxO4 and thus the first helix was chosen with regards to the solubility issues that may arise from longer peptides.

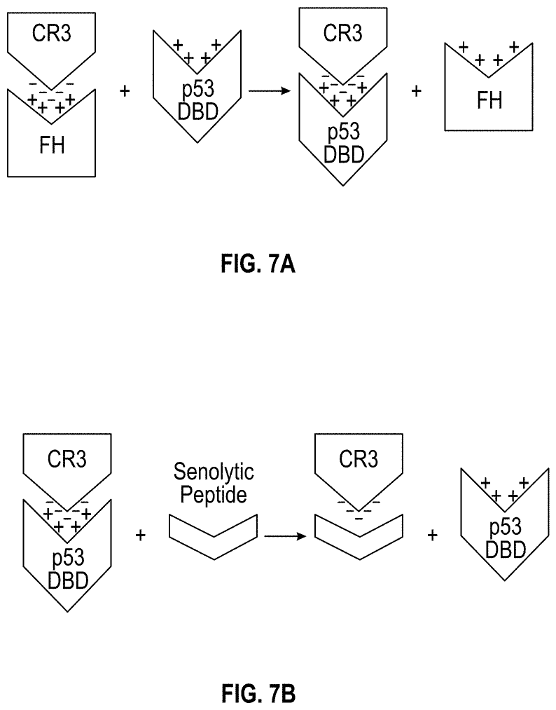

The NMR study findings are summarized in the FIG. 7A showing that the FH domain of FoxO4 interacts with the CR3 of FoxO4 from the same negatively charged surface that is used in the CR3 interaction with the p53DBD at a comparable affinity. FH domain of the FoxO4 was chosen as the template structure in the design of peptide inhibitor to block the CR3 domain of FoxO4 (FIG. 7B). The designed peptides based on the FH domain of FoxO4 might still harbor some of the functional roles that are played by the native FH domain This condition will lead to side-effects due to the interference with the other complexes formed by the FH domain of FoxO4.

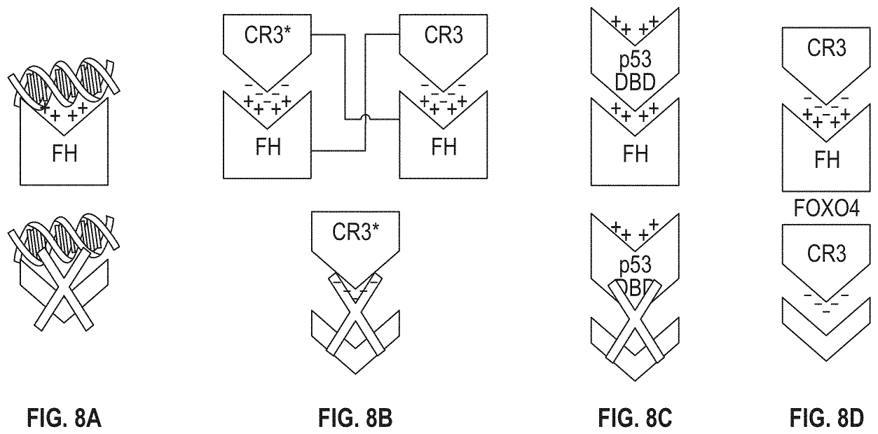

In some embodiments, the inventors proposed the selective inhibition of FoxO4 by designed peptides that show the maximum level of FoxO4 inhibition and also minimal side effects due to reduced interference by other interaction partners. To do so, all of the possible interacting partners of the designed peptides were analyzed (FIG. 8).

FoxOs are involved in many important cellular functions. DNA binding, as being one of the important functions of FoxO proteins, is modulated by the FH domain. Thus, any compounds that mimic FH domain of FoxO4 will also bind to FoxO consensus DNA, leading to interference with the DNA binding of FoxOs (FIG. 8A).

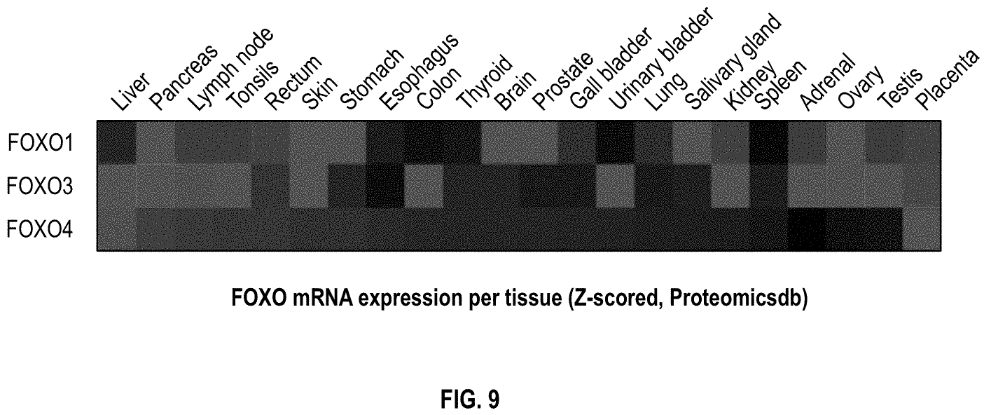

Said functional redundancy in FoxO members will lead to formation of hetero domain interactions in that FH domain of FoxO4 with the CR3 domain of other FoxOs (FIG. 8B), and thus will lead to inhibition of other FoxOs as well. The heat-maps from Proteomicdb compared the difference in FoxO1, 3, and 4 expressions in various tissues. According these expression profiles, FoxO4 in non-senescent cells is expressed only marginally throughout the body, while FoxO1 and FoxO3 expressions are higher in non-senescent cells that are found particularly in the tissues such as liver, pancreas, colon, ovaries, lymph nodes, and bladder (FIG. 9). This profile suggests that nonspecific inhibition of the CR3 domain of other FoxOs than FoxO4 will lead to undesired side effects in normal cells. Essentially, in vivo experiments using FoxO knockout mice showed that loss of these proteins lead to tumor growth. If one deletes more FoxOs (Compare FoxO1/Fox03/Fox04 mutant mice with FoxO3−/− mice), a more frequent, and faster (early in age) cancer growth was observed. According to another recent finding on FoxO3, the repression of which promoted survival pathways and metastasis of melanoma cells. Parallel to the paradigm that interference with p53 will lead to undesired consequences, any interferences with the other FoxOs will end up with unwanted outcomes such as skin tumors. Hence, to eliminate the risk of such consequences, the Senolytic Peptides, for example, the peptides shown in the SEQUENCE LISTING as SEQ ID NO: 1 through SEQ ID NO: 245, are further optimized to minimize their interaction with the other FoxOs, namely FoxO1 and FoxO3 was minimized (FIG. 8B).

The FH domain was also found to interact with the p53DBD although this interaction is relatively weak compared to the CR3 domain binding. Therefore, the designed peptides might also interfere with the p53 as well (FIG. 8D). Thus these potential interactions of the designed peptides were aimed to be at minimum to avoid undesired consequences. During design, the peptides were selected have a minimal interaction with the DNA, other FoxO members and also p53DBD, while its interaction with the CR3 of FoxO4 was maximized (FIG. 8E). In some embodiments, the Senolytic Peptides, for example, the peptides shown in the SEQUENCE LISTING as SEQ ID NO: 1 through SEQ ID NO: 245, were selected to be specific only to the CR3 of FoxO4, but not the other partners including p53DBD, DNA, FoxO1 and FoxO3.

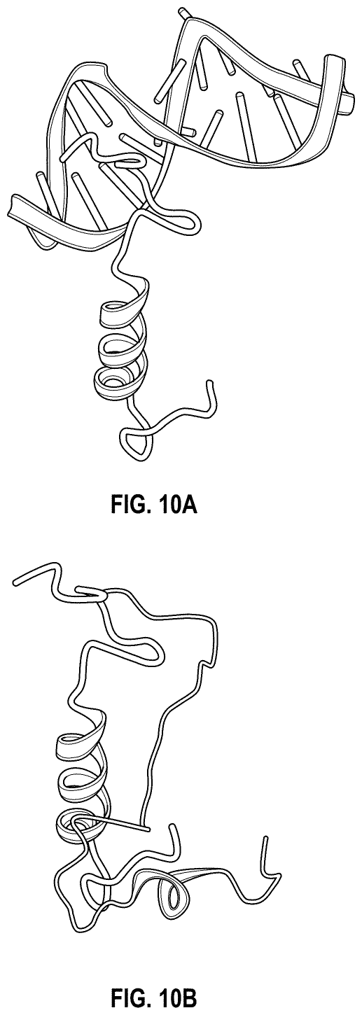

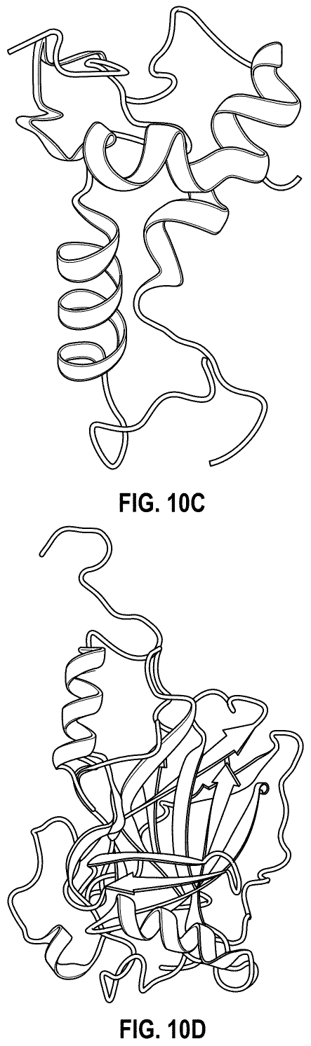

The N-terminal of FH domain of FoxO4 comprising the residues from 1 to 35 (FH-Nter¹⁻³⁵) were obtained from the model resolved in FIG. 1A. The FH-Nter¹⁻³⁵ was re-docked to DNA (FIG. 10A), CR3 of FoxO1 (FIG. 10B), the CR3 of FoxO3 (FIG. 10C), the p53DBD (FIG. 10D) and the CR3 domain of FoxO4 (FIG. 10E), using HADDOCK. The binding interface of all of these complexes were analyzed and the prediction for stabilizing mutations were decided for the FH-CR3 FoxO4 complex (FIG. 10E). For the rest of the complexes destabilizing or neutral mutations were selected.

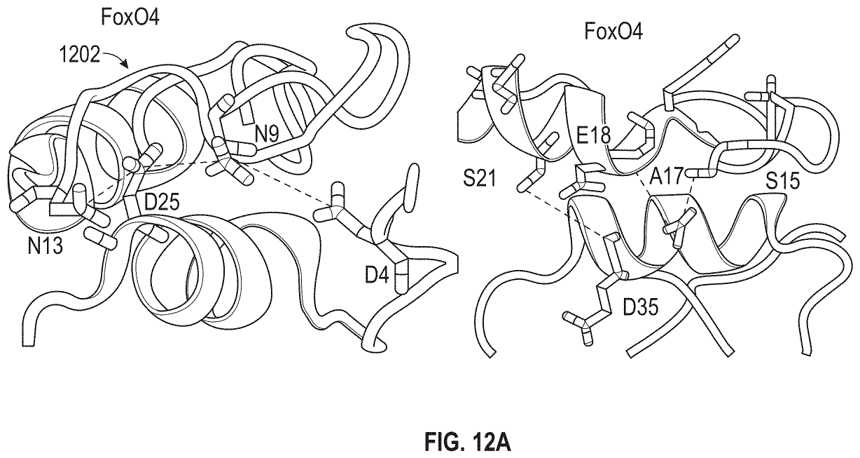

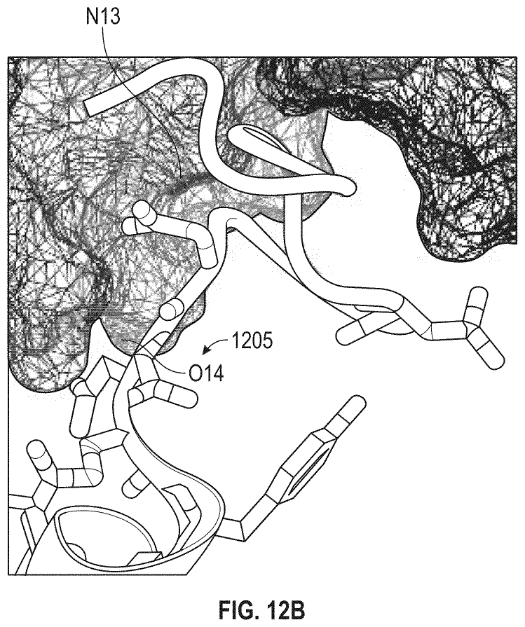

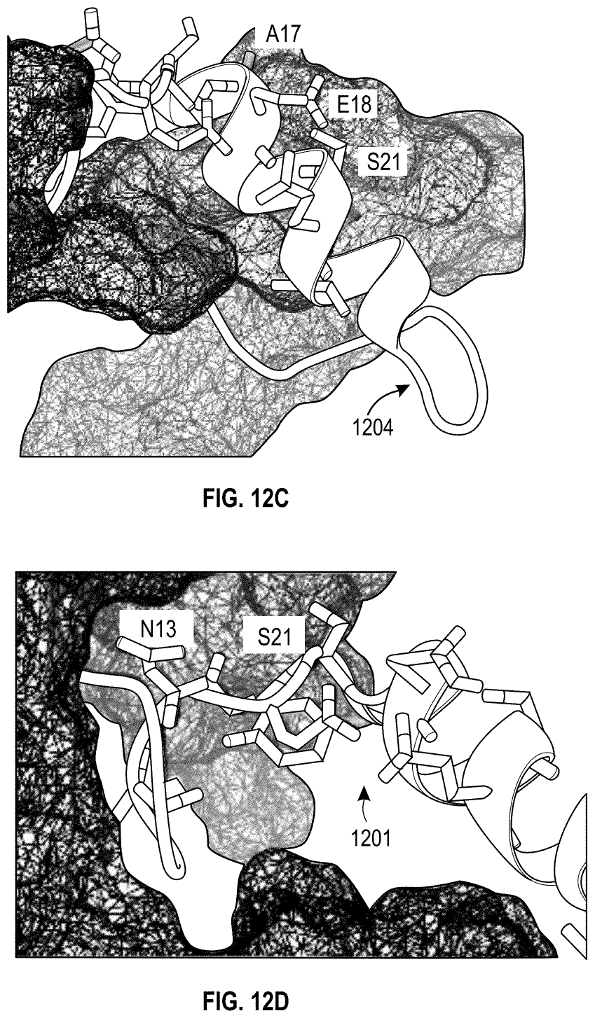

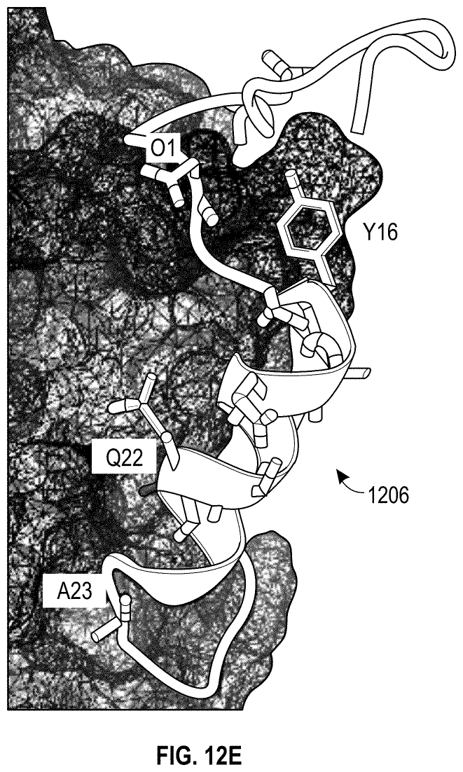

In some embodiments, to enhance the selectivity of the disclosed peptide(s) towards the CR3 domain of FoxO4, stabilizing mutations of the interactions between the FH domain of FoxO4 and the CR3 domain of FoxO4 (FIG. 10E) was selected. From this perspective, the interaction network between the FH and the CR of FoxO4 was revealed in the FIG. 12A. According to this network, the binding affinity of the designed peptide towards the CR3 of FoxO4 is to be enhanced by mutating S6, N9, N13, S15, A17, E18 and/or S21 to a positively charged amino acid such as K or R. The left panel in FIG. 12A depicts that if a positively charged amino acid is substituted at the positions of S6, N9 and N13, potential electrostatic interactions between any of the aspartic acids D4 or D25 and positively charged mutations at the positions of S6, N9 and N13 stabilize the complex. The right panel in FIG. 12A similarly shows that possible electrostatic interactions between the negatively charged amino acids of the CR3 domain (D22 and D35) and the positively charged mutations at the positions S15, A17, El 8 and/or S21 will enhance the binding. In some embodiments, the mutations present in the Senolytic Peptides disclosed herein, for example, the peptides shown in the SEQUENCE LISTING as SEQ ID NO: 1 through SEQ ID NO: 245, may be effective to maximize the specificity of the peptides towards FoxO4.

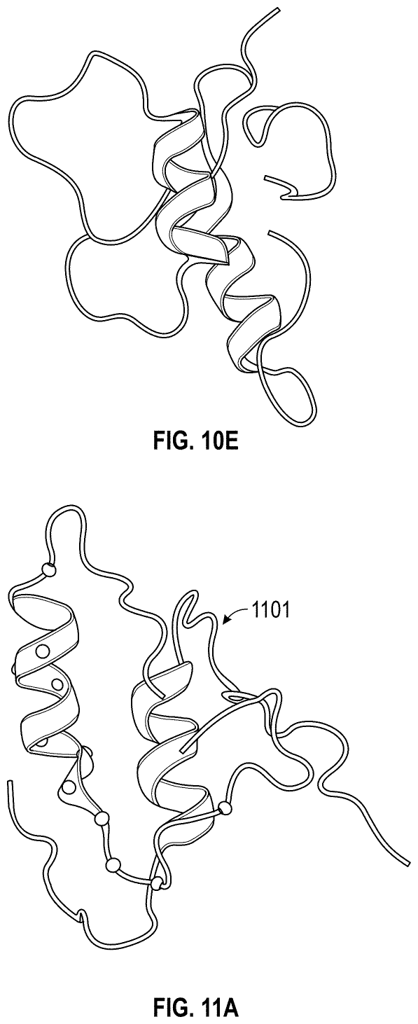

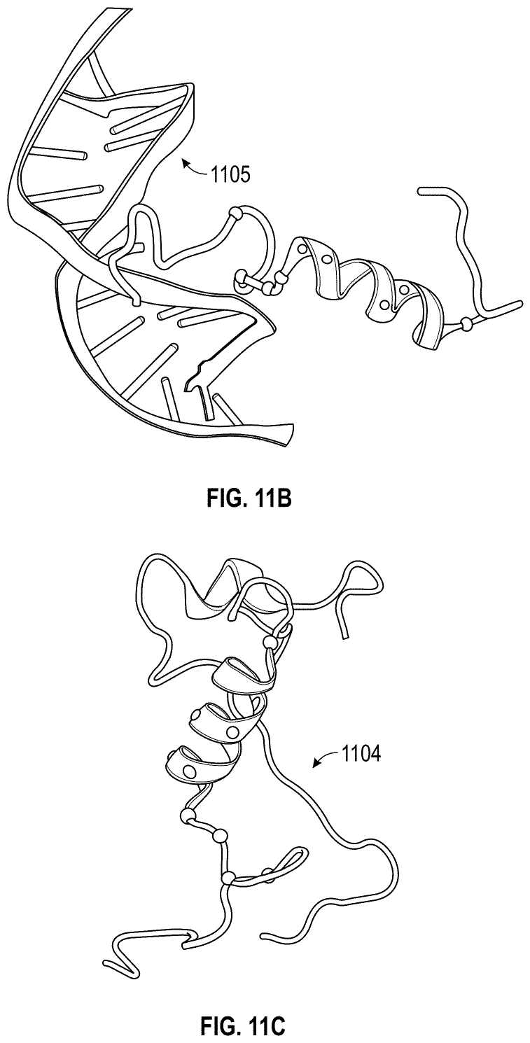

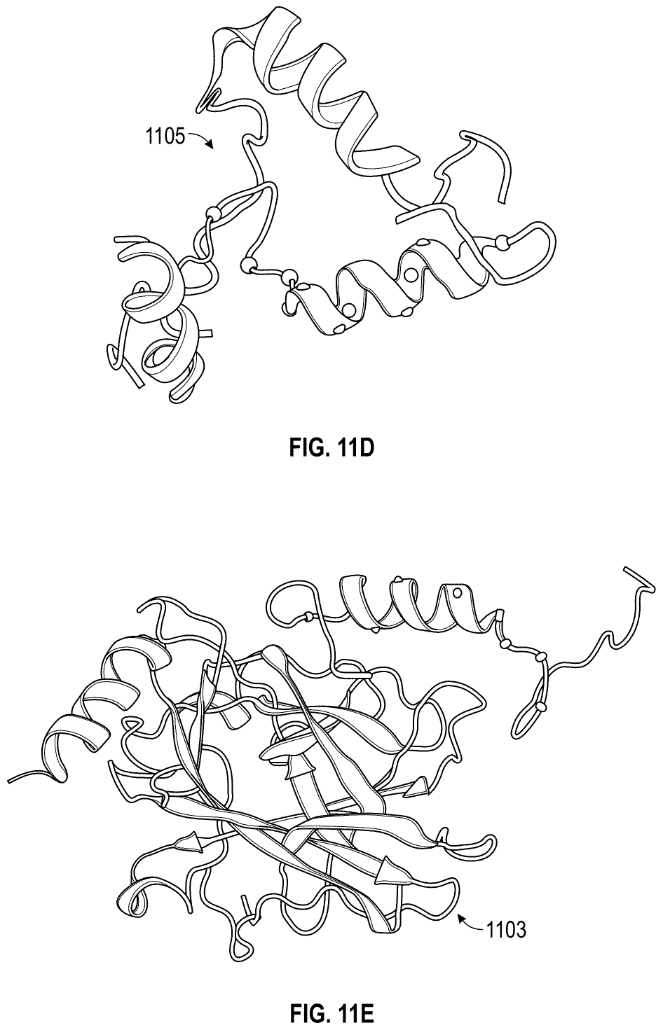

These positions were also investigated in all of the other complexes that the peptide can form with other partners such as DNA 1105, CR3 domain 1101 of other FoxOs (1104 and 1105) and also p53DBD 1103 (FIGS. 11A-E). Structural investigations demonstrated that, as N13 and Q14 was located at the binding interface of FH-Nter¹⁻³⁵ and DNA, mutating these to larger amino acids such as K or R will disrupt the binding of the peptide to DNA 1205 (FIG. 12B). Among the selected amino acids for mutation; A17, E18 and S21 are located to the binding interface of the FH-Nter¹⁻³⁵ and FoxO1 (FIG. 12C); N13 and S21 are found at the interface of the complex formed by FoxO3 (FIG. 12D); while Y16 and Q22 reside at the interface of the complex formed by the p53DBD (FIG. 12E). Similar to the conclusions drawn for the impact of the mutations on the DNA interaction with FH-Nter¹⁻³⁵ (FIG. 12B), mutations of these amino acids to positively charged amino acids, as in the Senolytic Peptide(s), will disrupt the complex interactions formed by DNA, CR3 domains of other FoxOs (FoxO1 and FoxO3) and p53DBD, while they will enhance the interactions formed by FoxO4.

The role of p53 in senescence has been well characterized. Although p53 is considered to be a canonical inducer of senescence, p53 was also found to suppress senescence by converting it to quiescence (a reversible form of senescence). Essentially, the results showed that the transactivation of p53 by the MDM2 inhibitor, nutlin 3, suppressed the senescence. This dual role of p53 as being both inducer and suppressor of senescence highlighted the importance of the transactivation function of p53 in blocking the senescence in cells.

The transactivation domain (TAD) is at its N terminus and important for interaction with transcriptional coactivators and corepressors. The TAD is composed of two homologous subdomains, TAD1 (residues 1-40) and TAD2 (residues 41-61), which both contain conserved Φ-X-X-Φ-Φ sequence motifs (Φ=hydrophobic and X=any amino acid) common to many proteins regulating transcription. The TAD is followed by a proline-rich region (residues 63-97) and then by the highly conserved DNA-binding domain (residues 102-292) that exhibits sequence-specific DNA binding. In its activated state, p53 is extensively modified in both the N- and C-terminal regions of the protein. These modifications, especially phosphorylation of serine and threonine residues in the N-terminal transactivation domain, affect p53 stability and activity by modulating the affinity of protein-protein interactions. These modifications can either positively or negatively affect p53 and add a second layer of complex regulation to the divergent interactions of the p53 transactivation domain.

Phosphorylation of p53 affects its retention in the nucleus, transcriptional activity, DNA binding, degradation, and interaction with co-activators. Essentially, phosphorylation of p53 on serine 46 determines promoter selection and whether apoptosis is attenuated or amplified. Phosphorylation of serine 46 has no effect on the interaction of p53 with MDM2, the observation which can also be inferred from the structural findings showing the MDM2 interaction with the TAD1 of p53 whereas S46 lies in the TAD2. This phosphorylation abrogates the autoregulatory feedback loop through which MDM2 inhibits the apoptotic function of p53 and induces PTEN, which favors apoptosis. Further, the genome-wide level of phosphorylations of the p53TAD, particularly at two positions, Serine 46 (pS46) located at TAD2 and Serine 15 (pS15) located at TAD1, was analyzed. Correspondingly, the findings showed that the extent of phosphorylated S46 of p53 bound to DNA is considerably higher in cells that are directed towards apoptosis, while the degree of phosphorylation at S15 remains indifferent. Overall, these results underscored the importance of the phosphorylated S46 in the p53TAD for apoptosis.

Given the finding stating that FoxO4 suppresses the apoptosis mediated by the p53 that is phosphorylated at S46 in senescent cells, the binding of FoxO4 to the p53TAD that is phosphorylated at S46 will be one of inhibitory action of FoxO4 on p53. Hence, liberation of the FoxO4, particularly from the surface on the p53TAD that is phosphorylated at S46 will circumvent this inhibitory action of FoxO4 on p53, liberating the apoptotic function of p53.

To model this interaction, the atomic structures of the p53 and FoxO4 was analyzed. The p53TAD is unstructured in solution. Nevertheless, a high resolution X-ray structure is available for the TAD2 which contains the S46. The structure is a complex structure showing the interaction of TAD2 with another protein partner that involved in DNA repair. Particularly, this domain TAD2 stood out with a number of the negatively charged amino acids such as aspartate and glutamate. Further phosphorylation at the Serine 46 will be an addition to the negative charges on p53TAD. Hence, in this highly negatively charged states S46 phosphorylated p53 will interact with the FH domain of FoxO4 which contain mostly positive charged amino acids.

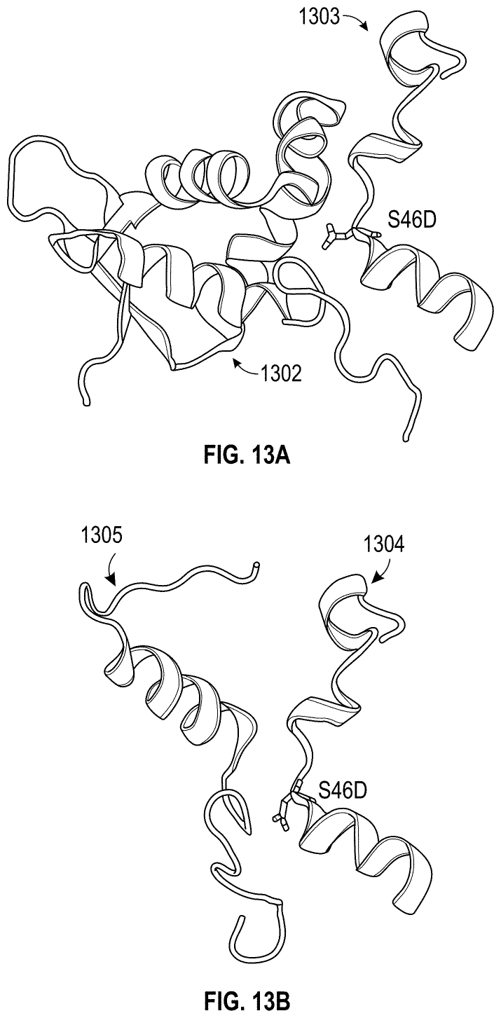

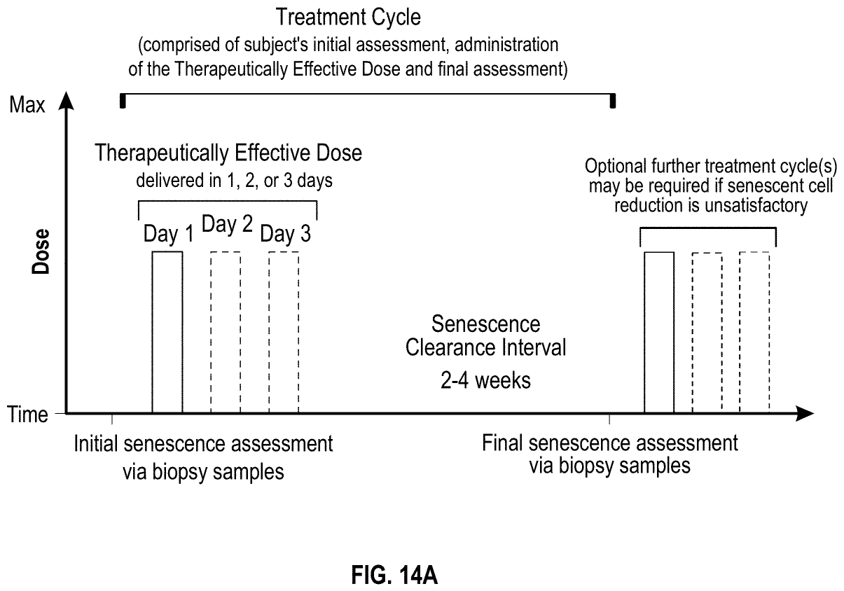

In the models, FH and the FH-Nter¹⁻³⁵ peptide was docked to the p53TAD structure retrieved from 2b3g (see FIG. 13A). The S46 in the structure was mutated to D (aspartate) as the phosphate mimic The docking results suggest that FH interacts with the surface of p53TAD2 that includes S46D as a phosphate mimic. Similarly, FH-Nter¹⁻³⁵ peptide interacts with the p53TAD2 in the same surface. It was clarified that FH domain of FoxO4 will occupy the transactivation domain of p53 when phosphorylated at S46. As similarly explained by the interference of p53DBD by the CR3 domain of FoxO4, the possible interaction of FH of FoxO4 1302with the S46 phosphorylated p53TAD 1303 (FIG. 13A) will correlate with the inhibition of the p53 mediated apoptosis. To interfere with this binding the FH-Nter¹⁻³⁵ peptide interaction with S46 phosphorylated p53TAD2 is analyzed (FIG. 13B). It has been shown that FH-Nter¹⁻³⁵ interacts with the S46 phosphorylated p53TAD from a smaller surface that also includes S46D. Owing to the smaller size of the FH-Nter¹⁻³⁵ than the FH domain of FoxO4, the steric blockage of p53TAD by the FH of FoxO4 will be reduced by the Senolytic Peptide(s). This steric blockage may occur in p53TAD2 subdomain and also may occur in other subdomains p53TAD1. The inhibitory action of FoxO4 on the p53 mediated apoptosis, may be explained by the said blockage of the p53TAD regions that are essential for activators and regulatory proteins by the FH domain. Hence, the inhibitory action of FoxO4 on the p53 mediated apoptosis, that will be resulted from the possible interaction between the FH of FoxO4 and the S46 phosphorylated p53TAD p53TAD will be eliminated by the Senolytic Peptide(s). From this perspective, the Senolytic Peptide(s), for example, shown in the SEQUENCE LISTING as SEQ ID NO: 1 through SEQ ID NO: 245, are applicable to also inhibit the plausible interaction between the FH and p53TAD. By replacing the FH, the Senolytic Peptide(s) might interact with p53TAD. The Senolytic Peptide(s) were optimized to have a comparable or enhanced binding affinity towards the S46 phosphorylated p53TAD. In some embodiments, the Senolytic Peptides my exhibit an increased or an unchanged interference with the p53TAD that is phosphorylated at the Serine 46 region, compared to that of the FH domain of the FoxO4.

In some embodiments, FoxO4 and p53 are proposed to interact from multiple domains. The said NMR study suggested that FoxO4 will bind to p53DBD through the CR3 domain. While in vivo findings showing that FoxO4 inhibits the apoptosis mediated by the S46 phosphorylated p53, along with the likelihood of strong electrostatic interactions between the FH domain of FoxO4, that contains positively charged amino acids, and the p53TAD, that contains negatively charged amino acids, implied that FoxO4 will also bind to p53TAD that is phosphorylated at S46 and restrains its apoptotic activity. The possible interactions between FoxO4 and p53, leading FoxO4 to inhibition of apoptosis mediated by S46 phosphorylated p53 in senescent cells, wherein the senescent cell expresses the senescence-associated secretory phenotype (SASP) were delineated. These interactions are formed between

(i) The CR3 of FoxO4 and the p53DBD, and

(ii) The FH of FoxO4 and the S46 phosphorylated p53TAD.

In some embodiments, the Senolytic Peptides, for example, the peptides shown in the SEQUENCE LISTING as SEQ ID NO: 1 through SEQ ID NO: 245, are designed to optimize these interactions, for example, to liberate the FoxO4 protein from p53. Revealed by the analysis of binding interfaces of the complexes in FIG. 11, the particular mutations of the FH-Nter¹⁻³⁵ present in the Senolytic Peptide(s), for example, included in the SEQUENCE LISTING as SEQ ID NO: 1 through SEQ ID NO: 245, may be effective to maximize the CR3 of FoxO4 interaction (FIG. 11A), while the same mutations will have destabilizing (or neutral) im acts on the interactions between the rest of the complexes (FIG. 11B-E). Concomitantly the mutations were selected to have stabilizing or neutral impacts on the binding of the FH-Nter¹⁻³⁵ to the p53TAD that is phosphorylated at S46.

In some embodiments, the Senolytic Peptides refer to rationally designed selective inhibitors of the FoxO4 interactions with the p53. In some embodiments, the peptides may be generally represented:

PRKGGRRRRAWGXRRXRXXXRRRRRRRPRKRLRLR

wherein “X” is one of the amino acids: R, H or K, and wherein any derived L-peptide is not a fragment of a native human protein. In some embodiments, the Senolytic Peptide is any amino acid sequence comprising any one of SEQ ID NO: 1 through SEQ ID NO: 245, which are reproduced herein in Table 2.

TABLE 2
Senolytic Peptide(s).

| | |
|---|---|
| SEQ ID NO: 1 | PRKGGRRRRA WGHRRHRHHH RRRRRRRPRK |
| | RLRLR |
| SEQ ID NO: 2 | PRKGGRRRRA WGKRRKRKKK RRRRRRRPRK |
| | RLRLR |
| SEQ ID NO: 3 | PRKGGRRRRA WGRRRHRHKK RRRRRRRPRK |
| | RLRLR |
| SEQ ID NO: 4 | PRKGGRRRRA WGRRRHRRHH RRRRRRRPRK |
| | RLRLR |
| SEQ ID NO: 5 | PRKGGRRRRA WGRRRKRRHH RRRRRRRPRK |
| | RLRLR |
| SEQ ID NO: 6 | PRKGGRRRRA WGRRRKRRHK RRRRRRRPRK |
| | RLRLR |
| SEQ ID NO: 7 | PRKGGRRRRA WGRRRKRRKK RRRRRRRPRK |
| | RLRLR |
| SEQ ID NO: 8 | PRKGGRRRRA WGRRRRRRHK RRRRRRRPRK |
| | RLRLR |
| SEQ ID NO: 9 | PRKGGRRRRA WGRRRRRRRK RRRRRRRPRK |
| | RLRLR |
| SEQ ID NO: 10 | PRKGGRRRRA WGRRRRRRRR RRRRRRRPRK |
| | RLRLR |
| SEQ ID NO: 11 | dRPRKGGRRRR AWGRRRRRRR RRRRRRRAPR |
| | KRLTL |
| SEQ ID NO: 12 | PRKGGSRRRA WGNQRYARLI RQAIESAPEK |
| | RLTLA |
| SEQ ID NO: 13 | PRKGGRRRRA WGHRRHRHHK RRRRRRRPRK |
| | RLRLR |
| SEQ ID NO: 14 | PRKGGRRRRA WGHRRHRHHR RRRRRRRPRK |
| | RLRLR |
| SEQ ID NO: 15 | PRKGGRRRRA WGHRRHRHKH RRRRRRRPRK |
| | RLRLR |
| SEQ ID NO: 16 | PRKGGRRRRA WGHRRHRHKK RRRRRRRPRK |
| | RLRLR |
| SEQ ID NO: 17 | PRKGGRRRRA WGHRRHRHKR RRRRRRRPRK |
| | RLRLR |
| SEQ ID NO: 18 | PRKGGRRRRA WGHRRHRHRH RRRRRRRPRK |
| | RLRLR |
| SEQ ID NO: 19 | PRKGGRRRRA WGHRRHRHRK RRRRRRRPRK |
| | RLRLR |
| SEQ ID NO: 20 | PRKGGRRRRA WGHRRHRHRR RRRRRRRPRK |
| | RLRLR |
| SEQ ID NO: 21 | PRKGGRRRRA WGHRRHRKHH RRRRRRRPRK |
| | RLRLR |
| SEQ ID NO: 22 | PRKGGRRRRA WGHRRHRKHK RRRRRRRPRK |
| | RLRLR |
| SEQ ID NO: 23 | PRKGGRRRRA WGHRRHRKHR RRRRRRRPRK |
| | RLRLR |
| SEQ ID NO: 24 | PRKGGRRRRA WGHRRHRKKH RRRRRRRPRK |
| | RLRLR |
| SEQ ID NO: 25 | PRKGGRRRRA WGHRRHRKKK RRRRRRRPRK |
| | RLRLR |
| SEQ ID NO: 26 | PRKGGRRRRA WGHRRHRKKR RRRRRRRPRK |
| | RLRLR |
| SEQ ID NO: 27 | PRKGGRRRRA WGHRRHRKRH RRRRRRRPRK |
| | RLRLR |
| SEQ ID NO: 28 | PRKGGRRRRA WGHRRHRKRK RRRRRRRPRK |
| | RLRLR |
| SEQ ID NO: 29 | PRKGGRRRRA WGHRRHRKRR RRRRRRRPRK |
| | RLRLR |
| SEQ ID NO: 30 | PRKGGRRRRA WGHRRHRRHH RRRRRRRPRK |
| | RLRLR |
| SEQ ID NO: 31 | PRKGGRRRRA WGHRRHRRHK RRRRRRRPRK |
| | RLRLR |
| SEQ ID NO: 32 | PRKGGRRRRA WGHRRHRRHR RRRRRRRPRK |
| | RLRLR |
| SEQ ID NO: 33 | PRKGGRRRRA WGHRRHRRKH RRRRRRRPRK |
| | RLRLR |
| SEQ ID NO: 34 | PRKGGRRRRA WGHRRHRRKK RRRRRRRPRK |
| | RLRLR |
| SEQ ID NO: 35 | PRKGGRRRRA WGHRRHRRKR RRRRRRRPRK |
| | RLRLR |
| SEQ ID NO: 36 | PRKGGRRRRA WGHRRHRRRH RRRRRRRPRK |
| | RLRLR |
| SEQ ID NO: 37 | PRKGGRRRRA WGHRRHRRRK RRRRRRRPRK |
| | RLRLR |
| SEQ ID NO: 38 | PRKGGRRRRA WGHRRHRRRR RRRRRRRPRK |
| | RLRLR |
| SEQ ID NO: 39 | PRKGGRRRRA WGHRRKRHHH RRRRRRRPRK |
| | RLRLR |
| SEQ ID NO: 40 | PRKGGRRRRA WGHRRKRHHK RRRRRRRPRK |
| | RLRLR |
| SEQ ID NO: 41 | PRKGGRRRRA WGHRRKRHHR RRRRRRRPRK |
| | RLRLR |
| SEQ ID NO: 42 | PRKGGRRRRA WGHRRKRHKH RRRRRRRPRK |
| | RLRLR |
| SEQ ID NO: 43 | PRKGGRRRRA WGHRRKRHKK RRRRRRRPRK |
| | RLRLR |
| SEQ ID NO: 44 | PRKGGRRRRA WGHRRKRHKR RRRRRRRPRK |
| | RLRLR |
| SEQ ID NO: 45 | PRKGGRRRRA WGHRRKRHRH RRRRRRRPRK |
| | RLRLR |
| SEQ ID NO: 46 | PRKGGRRRRA WGHRRKRHRK RRRRRRRPRK |
| | RLRLR |
| SEQ ID NO: 47 | PRKGGRRRRA WGHRRKRHRR RRRRRRRPRK |
| | RLRLR |
| SEQ ID NO: 48 | PRKGGRRRRA WGHRRKRKHH RRRRRRRPRK |
| | RLRLR |
| SEQ ID NO: 49 | PRKGGRRRRA WGHRRKRKHK RRRRRRRPRK |
| | RLRLR |
| SEQ ID NO: 50 | PRKGGRRRRA WGHRRKRKHR RRRRRRRPRK |
| | RLRLR |
| SEQ ID NO: 51 | PRKGGRRRRA WGHRRKRKKH RRRRRRRPRK |
| | RLRLR |
| SEQ ID NO: 52 | PRKGGRRRRA WGHRRKRKKK RRRRRRRPRK |
| | RLRLR |
| SEQ ID NO: 53 | PRKGGRRRRA WGHRRKRKKR RRRRRRRPRK |
| | RLRLR |
| SEQ ID NO: 54 | PRKGGRRRRA WGHRRKRKRH RRRRRRRPRK |
| | RLRLR |
| SEQ ID NO: 55 | PRKGGRRRRA WGHRRKRKRK RRRRRRRPRK |
| | RLRLR |
| SEQ ID NO: 56 | PRKGGRRRRA WGHRRKRKRR RRRRRRRPRK |
| | RLRLR |
| SEQ ID NO: 57 | PRKGGRRRRA WGHRRKRRHH RRRRRRRPRK |
| | RLRLR |
| SEQ ID NO: 58 | PRKGGRRRRA WGHRRKRRHK RRRRRRRPRK |
| | RLRLR |
| SEQ ID NO: 59 | PRKGGRRRRA WGHRRKRRHR RRRRRRRPRK |
| | RLRLR |
| SEQ ID NO: 60 | PRKGGRRRRA WGHRRKRRKH RRRRRRRPRK |
| | RLRLR |
| SEQ ID NO: 61 | PRKGGRRRRA WGHRRKRRKK RRRRRRRPRK |
| | RLRLR |
| SEQ ID NO: 62 | PRKGGRRRRA WGHRRKRRKR RRRRRRRPRK |
| | RLRLR |
| SEQ ID NO: 63 | PRKGGRRRRA WGHRRKRRRH RRRRRRRPRK |
| | RLRLR |
| SEQ ID NO: 64 | PRKGGRRRRA WGHRRKRRRK RRRRRRRPRK |
| | RLRLR |
| SEQ ID NO: 65 | PRKGGRRRRA WGHRRKRRRR RRRRRRRPRK |
| | RLRLR |
| SEQ ID NO: 66 | PRKGGRRRRA WGHRRRRHHH RRRRRRRPRK |
| | RLRLR |
| SEQ ID NO: 67 | PRKGGRRRRA WGHRRRRHHK RRRRRRRPRK |
| | RLRLR |
| SEQ ID NO: 68 | PRKGGRRRRA WGHRRRRHHR RRRRRRRPRK |
| | RLRLR |
| SEQ ID NO: 69 | PRKGGRRRRA WGHRRRRHKH RRRRRRRPRK |
| | RLRLR |
| SEQ ID NO: 70 | PRKGGRRRRA WGHRRRRHKK RRRRRRRPRK |
| | RLRLR |
| SEQ ID NO: 71 | PRKGGRRRRA WGHRRRRHKR RRRRRRRPRK |
| | RLRLR |
| SEQ ID NO: 72 | PRKGGRRRRA WGHRRRRHRH RRRRRRRPRK |
| | RLRLR |
| SEQ ID NO: 73 | PRKGGRRRRA WGHRRRRHRK RRRRRRRPRK |
| | RLRLR |
| SEQ ID NO: 74 | PRKGGRRRRA WGHRRRRHRR RRRRRRRPRK |
| | RLRLR |
| SEQ ID NO: 75 | PRKGGRRRRA WGHRRRRKHH RRRRRRRPRK |
| | RLRLR |
| SEQ ID NO: 76 | PRKGGRRRRA WGHRRRRKHK RRRRRRRPRK |
| | RLRLR |
| SEQ ID NO: 77 | PRKGGRRRRA WGHRRRRKHR RRRRRRRPRK |
| | RLRLR |
| SEQ ID NO: 78 | PRKGGRRRRA WGHRRRRKKH RRRRRRRPRK |
| | RLRLR |
| SEQ ID NO: 79 | PRKGGRRRRA WGHRRRRKKK RRRRRRRPRK |
| | RLRLR |
| SEQ ID NO: 80 | PRKGGRRRRA WGHRRRRKKR RRRRRRRPRK |
| | RLRLR |
| SEQ ID NO: 81 | PRKGGRRRRA WGHRRRRKRH RRRRRRRPRK |
| | RLRLR |
| SEQ ID NO: 82 | PRKGGRRRRA WGHRRRRKRK RRRRRRRPRK |
| | RLRLR |
| SEQ ID NO: 83 | PRKGGRRRRA WGHRRRRKRR RRRRRRRPRK |
| | RLRLR |
| SEQ ID NO: 84 | PRKGGRRRRA WGHRRRRRHH RRRRRRRPRK |
| | RLRLR |
| SEQ ID NO: 85 | PRKGGRRRRA WGHRRRRRHK RRRRRRRPRK |
| | RLRLR |
| SEQ ID NO: 86 | PRKGGRRRRA WGHRRRRRHR RRRRRRRPRK |
| | RLRLR |
| SEQ ID NO: 87 | PRKGGRRRRA WGHRRRRRKH RRRRRRRPRK |
| | RLRLR |
| SEQ ID NO: 88 | PRKGGRRRRA WGHRRRRRKK RRRRRRRPRK |
| | RLRLR |
| SEQ ID NO: 89 | PRKGGRRRRA WGHRRRRRKR RRRRRRRPRK |
| | RLRLR |
| SEQ ID NO: 90 | PRKGGRRRRA WGHRRRRRRH RRRRRRRPRK |
| | RLRLR |
| SEQ ID NO: 91 | PRKGGRRRRA WGHRRRRRRK RRRRRRRPRK |
| | RLRLR |
| SEQ ID NO: 92 | PRKGGRRRRA WGHRRRRRRR RRRRRRRPRK |
| | RLRLR |
| SEQ ID NO: 93 | PRKGGRRRRA WGKRRHRHHH RRRRRRRPRK |
| | RLRLR |
| SEQ ID NO: 94 | PRKGGRRRRA WGKRRHRHHK RRRRRRRPRK |
| | RLRLR |
| SEQ ID NO: 95 | PRKGGRRRRA WGKRRHRHHR RRRRRRRPRK |
| | RLRLR |
| SEQ ID NO: 96 | PRKGGRRRRA WGKRRHRHKH RRRRRRRPRK |
| | RLRLR |
| SEQ ID NO: 97 | PRKGGRRRRA WGKRRHRHKK RRRRRRRPRK |
| | RLRLR |
| SEQ ID NO: 98 | PRKGGRRRRA WGKRRHRHKR RRRRRRRPRK |
| | RLRLR |
| SEQ ID NO: 99 | PRKGGRRRRA WGKRRHRHRH RRRRRRRPRK |
| | RLRLR |
| SEQ ID NO: 100 | PRKGGRRRRA WGKRRHRHRK RRRRRRRPRK |
| | RLRLR |
| SEQ ID NO: 101 | PRKGGRRRRA WGKRRHRHRR RRRRRRRPRK |
| | RLRLR |
| SEQ ID NO: 102 | PRKGGRRRRA WGKRRHRKHH RRRRRRRPRK |
| | RLRLR |
| SEQ ID NO: 103 | PRKGGRRRRA WGKRRHRKHK RRRRRRRPRK |
| | RLRLR |
| SEQ ID NO: 104 | PRKGGRRRRA WGKRRHRKHR RRRRRRRPRK |
| | RLRLR |
| SEQ ID NO: 105 | PRKGGRRRRA WGKRRHRKKH RRRRRRRPRK |
| | RLRLR |
| SEQ ID NO: 106 | PRKGGRRRRA WGKRRHRKKK RRRRRRRPRK |
| | RLRLR |
| SEQ ID NO: 107 | PRKGGRRRRA WGKRRHRKKR RRRRRRRPRK |
| | RLRLR |
| SEQ ID NO: 108 | PRKGGRRRRA WGKRRHRKRH RRRRRRRPRK |
| | RLRLR |
| SEQ ID NO: 109 | PRKGGRRRRA WGKRRHRKRK RRRRRRRPRK |
| | RLRLR |
| SEQ ID NO: 110 | PRKGGRRRRA WGKRRHRKRR RRRRRRRPRK |
| | RLRLR |
| SEQ ID NO: 111 | PRKGGRRRRA WGKRRHRRHH RRRRRRRPRK |
| | RLRLR |
| SEQ ID NO: 112 | PRKGGRRRRA WGKRRHRRHK RRRRRRRPRK |
| | RLRLR |
| SEQ ID NO: 113 | PRKGGRRRRA WGKRRHRRHR RRRRRRRPRK |
| | RLRLR |
| SEQ ID NO: 114 | PRKGGRRRRA WGKRRHRRKH RRRRRRRPRK |
| | RLRLR |
| SEQ ID NO: 115 | PRKGGRRRRA WGKRRHRRKK RRRRRRRPRK |
| | RLRLR |
| SEQ ID NO: 116 | PRKGGRRRRA WGKRRHRRKR RRRRRRRPRK |
| | RLRLR |
| SEQ ID NO: 117 | PRKGGRRRRA WGKRRHRRRH RRRRRRRPRK |
| | RLRLR |
| SEQ ID NO: 118 | PRKGGRRRRA WGKRRHRRRK RRRRRRRPRK |
| | RLRLR |
| SEQ ID NO: 119 | PRKGGRRRRA WGKRRHRRRR RRRRRRRPRK |
| | RLRLR |
| SEQ ID NO: 120 | PRKGGRRRRA WGKRRKRHHH RRRRRRRPRK |
| | RLRLR |
| SEQ ID NO: 121 | PRKGGRRRRA WGKRRKRHHK RRRRRRRPRK |
| | RLRLR |
| SEQ ID NO: 122 | PRKGGRRRRA WGKRRKRHHR RRRRRRRPRK |
| | RLRLR |
| SEQ ID NO: 123 | PRKGGRRRRA WGKRRKRHKH RRRRRRRPRK |
| | RLRLR |
| SEQ ID NO: 124 | PRKGGRRRRA WGKRRKRHKK RRRRRRRPRK |
| | RLRLR |
| SEQ ID NO: 125 | PRKGGRRRRA WGKRRKRHKR RRRRRRRPRK |
| | RLRLR |
| SEQ ID NO: 126 | PRKGGRRRRA WGKRRKRHRH RRRRRRRPRK |
| | RLRLR |
| SEQ ID NO: 127 | PRKGGRRRRA WGKRRKRHRK RRRRRRRPRK |
| | RLRLR |
| SEQ ID NO: 128 | PRKGGRRRRA WGKRRKRHRR RRRRRRRPRK |
| | RLRLR |
| SEQ ID NO: 129 | PRKGGRRRRA WGKRRKRKHH RRRRRRRPRK |
| | RLRLR |
| SEQ ID NO: 130 | PRKGGRRRRA WGKRRKRKHK RRRRRRRPRK |
| | RLRLR |
| SEQ ID NO: 131 | PRKGGRRRRA WGKRRKRKHR RRRRRRRPRK |
| | RLRLR |
| SEQ ID NO: 132 | PRKGGRRRRA WGKRRKRKKH RRRRRRRPRK |
| | RLRLR |
| SEQ ID NO: 133 | PRKGGRRRRA WGKRRKRKKR RRRRRRRPRK |
| | RLRLR |
| SEQ ID NO: 134 | PRKGGRRRRA WGKRRKRKRH RRRRRRRPRK |
| | RLRLR |
| SEQ ID NO: 135 | PRKGGRRRRA WGKRRKRKRK RRRRRRRPRK |
| | RLRLR |
| SEQ ID NO: 136 | PRKGGRRRRA WGKRRKRKRR RRRRRRRPRK |
| | RLRLR |
| SEQ ID NO: 137 | PRKGGRRRRA WGKRRKRRHH RRRRRRRPRK |
| | RLRLR |
| SEQ ID NO: 138 | PRKGGRRRRA WGKRRKRRHK RRRRRRRPRK |
| | RLRLR |
| SEQ ID NO: 139 | PRKGGRRRRA WGKRRKRRHR RRRRRRRPRK |
| | RLRLR |
| SEQ ID NO: 140 | PRKGGRRRRA WGKRRKRRKH RRRRRRRPRK |
| | RLRLR |
| SEQ ID NO: 141 | PRKGGRRRRA WGKRRKRRKK RRRRRRRPRK |
| | RLRLR |
| SEQ ID NO: 142 | PRKGGRRRRA WGKRRKRRKR RRRRRRRPRK |
| | RLRLR |
| SEQ ID NO: 143 | PRKGGRRRRA WGKRRKRRRH RRRRRRRPRK |
| | RLRLR |
| SEQ ID NO: 144 | PRKGGRRRRA WGKRRKRRRK RRRRRRRPRK |
| | RLRLR |
| SEQ ID NO: 145 | PRKGGRRRRA WGKRRKRRRR RRRRRRRPRK |
| | RLRLR |
| SEQ ID NO: 146 | PRKGGRRRRA WGKRRRRHHH RRRRRRRPRK |
| | RLRLR |
| SEQ ID NO: 147 | PRKGGRRRRA WGKRRRRHHK RRRRRRRPRK |
| | RLRLR |
| SEQ ID NO: 148 | PRKGGRRRRA WGKRRRRHHR RRRRRRRPRK |
| | RLRLR |
| SEQ ID NO: 149 | PRKGGRRRRA WGKRRRRHKH RRRRRRRPRK |
| | RLRLR |
| SEQ ID NO: 150 | PRKGGRRRRA WGKRRRRHKK RRRRRRRPRK |
| | RLRLR |
| SEQ ID NO: 151 | PRKGGRRRRA WGKRRRRHKR RRRRRRRPRK |
| | RLRLR |
| SEQ ID NO: 152 | PRKGGRRRRA WGKRRRRHRH RRRRRRRPRK |
| | RLRLR |
| SEQ ID NO: 153 | PRKGGRRRRA WGKRRRRHRK RRRRRRRPRK |
| | RLRLR |
| SEQ ID NO: 154 | PRKGGRRRRA WGKRRRRHRR RRRRRRRPRK |
| | RLRLR |
| SEQ ID NO: 155 | PRKGGRRRRA WGKRRRRKHH RRRRRRRPRK |
| | RLRLR |
| SEQ ID NO: 156 | PRKGGRRRRA WGKRRRRKHK RRRRRRRPRK |
| | RLRLR |
| SEQ ID NO: 157 | PRKGGRRRRA WGKRRRRKHR RRRRRRRPRK |
| | RLRLR |
| SEQ ID NO: 158 | PRKGGRRRRA WGKRRRRKKH RRRRRRRPRK |
| | RLRLR |
| SEQ ID NO: 159 | PRKGGRRRRA WGKRRRRKKK RRRRRRRPRK |
| | RLRLR |
| SEQ ID NO: 160 | PRKGGRRRRA WGKRRRRKKR RRRRRRRPRK |
| | RLRLR |
| SEQ ID NO: 161 | PRKGGRRRRA WGKRRRRKRH RRRRRRRPRK |
| | RLRLR |
| SEQ ID NO: 162 | PRKGGRRRRA WGKRRRRKRK RRRRRRRPRK |
| | RLRLR |
| SEQ ID NO: 163 | PRKGGRRRRA WGKRRRRKRR RRRRRRRPRK |
| | RLRLR |
| SEQ ID NO: 164 | PRKGGRRRRA WGKRRRRRHH RRRRRRRPRK |
| | RLRLR |
| SEQ ID NO: 165 | PRKGGRRRRA WGKRRRRRHK RRRRRRRPRK |
| | RLRLR |
| SEQ ID NO: 166 | PRKGGRRRRA WGKRRRRRHR RRRRRRRPRK |
| | RLRLR |
| SEQ ID NO: 167 | PRKGGRRRRA WGKRRRRRKH RRRRRRRPRK |
| | RLRLR |
| SEQ ID NO: 168 | PRKGGRRRRA WGKRRRRRKK RRRRRRRPRK |
| | RLRLR |
| SEQ ID NO: 169 | PRKGGRRRRA WGKRRRRRKR RRRRRRRPRK |
| | RLRLR |
| SEQ ID NO: 170 | PRKGGRRRRA WGKRRRRRRH RRRRRRRPRK |
| | RLRLR |
| SEQ ID NO: 171 | PRKGGRRRRA WGKRRRRRRK RRRRRRRPRK |
| | RLRLR |
| SEQ ID NO: 172 | PRKGGRRRRA WGKRRRRRRR RRRRRRRPRK |
| | RLRLR |
| SEQ ID NO: 173 | PRKGGRRRRA WGRRRHRHHH RRRRRRRPRK |
| | RLRLR |
| SEQ ID NO: 174 | PRKGGRRRRA WGRRRHRHHK RRRRRRRPRK |
| | RLRLR |
| SEQ ID NO: 175 | PRKGGRRRRA WGRRRHRHHR RRRRRRRPRK |
| | RLRLR |
| SEQ ID NO: 176 | PRKGGRRRRA WGRRRHRHKH RRRRRRRPRK |
| | RLRLR |
| SEQ ID NO: 177 | PRKGGRRRRA WGRRRHRHKR RRRRRRRPRK |
| | RLRLR |
| SEQ ID NO: 178 | PRKGGRRRRA WGRRRHRHRH RRRRRRRPRK |
| | RLRLR |
| SEQ ID NO: 179 | PRKGGRRRRA WGRRRHRHRK RRRRRRRPRK |
| | RLRLR |
| SEQ ID NO: 180 | PRKGGRRRRA WGRRRHRHRR RRRRRRRPRK |
| | RLRLR |
| SEQ ID NO: 181 | PRKGGRRRRA WGRRRHRKHH RRRRRRRPRK |
| | RLRLR |
| SEQ ID NO: 182 | PRKGGRRRRA WGRRRHRKHK RRRRRRRPRK |
| | RLRLR |
| SEQ ID NO: 183 | PRKGGRRRRA WGRRRHRKHR RRRRRRRPRK |
| | RLRLR |
| SEQ ID NO: 184 | PRKGGRRRRA WGRRRHRKKH RRRRRRRPRK |
| | RLRLR |
| SEQ ID NO: 185 | PRKGGRRRRA WGRRRHRKKK RRRRRRRPRK |
| | RLRLR |
| SEQ ID NO: 186 | PRKGGRRRRA WGRRRHRKKR RRRRRRRPRK |
| | RLRLR |
| SEQ ID NO: 187 | PRKGGRRRRA WGRRRHRKRH RRRRRRRPRK |
| | RLRLR |
| SEQ ID NO: 188 | PRKGGRRRRA WGRRRHRKRK RRRRRRRPRK |
| | RLRLR |
| SEQ ID NO: 189 | PRKGGRRRRA WGRRRHRKRR RRRRRRRPRK |
| | RLRLR |
| SEQ ID NO: 190 | PRKGGRRRRA WGRRRHRRHK RRRRRRRPRK |
| | RLRLR |
| SEQ ID NO: 191 | PRKGGRRRRA WGRRRHRRHR RRRRRRRPRK |
| | RLRLR |
| SEQ ID NO: 192 | PRKGGRRRRA WGRRRHRRKH RRRRRRRPRK |
| | RLRLR |
| SEQ ID NO: 193 | PRKGGRRRRA WGRRRHRRKK RRRRRRRPRK |
| | RLRLR |
| SEQ ID NO: 194 | PRKGGRRRRA WGRRRHRRKR RRRRRRRPRK |
| | RLRLR |
| SEQ ID NO: 195 | PRKGGRRRRA WGRRRHRRRH RRRRRRRPRK |
| | RLRLR |
| SEQ ID NO: 196 | PRKGGRRRRA WGRRRHRRRK RRRRRRRPRK |
| | RLRLR |
| SEQ ID NO: 197 | PRKGGRRRRA WGRRRHRRRR RRRRRRRPRK |
| | RLRLR |
| SEQ ID NO: 198 | PRKGGRRRRA WGRRRKRHHH RRRRRRRPRK |
| | RLRLR |
| SEQ ID NO: 199 | PRKGGRRRRA WGRRRKRHHK RRRRRRRPRK |
| | RLRLR |
| SEQ ID NO: 200 | PRKGGRRRRA WGRRRKRHHR RRRRRRRPRK |
| | RLRLR |
| SEQ ID NO: 201 | PRKGGRRRRA WGRRRKRHKH RRRRRRRPRK |
| | RLRLR |
| SEQ ID NO: 202 | PRKGGRRRRA WGRRRKRHKK RRRRRRRPRK |
| | RLRLR |
| SEQ ID NO: 203 | PRKGGRRRRA WGRRRKRHKR RRRRRRRPRK |
| | RLRLR |
| SEQ ID NO: 204 | PRKGGRRRRA WGRRRKRHRH RRRRRRRPRK |
| | RLRLR |
| SEQ ID NO: 205 | PRKGGRRRRA WGRRRKRHRK RRRRRRRPRK |
| | RLRLR |
| SEQ ID NO: 206 | PRKGGRRRRA WGRRRKRHRR RRRRRRRPRK |
| | RLRLR |
| SEQ ID NO: 207 | PRKGGRRRRA WGRRRKRKHH RRRRRRRPRK |
| | RLRLR |
| SEQ ID NO: 208 | PRKGGRRRRA WGRRRKRKHK RRRRRRRPRK |
| | RLRLR |
| SEQ ID NO: 209 | PRKGGRRRRA WGRRRKRKHR RRRRRRRPRK |
| | RLRLR |
| SEQ ID NO: 210 | PRKGGRRRRA WGRRRKRKKH RRRRRRRPRK |
| | RLRLR |
| SEQ ID NO: 211 | PRKGGRRRRA WGRRRKRKKK RRRRRRRPRK |
| | RLRLR |
| SEQ ID NO: 212 | PRKGGRRRRA WGRRRKRKKR RRRRRRRPRK |
| | RLRLR |
| SEQ ID NO: 213 | PRKGGRRRRA WGRRRKRKRH RRRRRRRPRK |
| | RLRLR |
| SEQ ID NO: 214 | PRKGGRRRRA WGRRRKRKRK RRRRRRRPRK |
| | RLRLR |
| SEQ ID NO: 215 | PRKGGRRRRA WGRRRKRKRR RRRRRRRPRK |
| | RLRLR |
| SEQ ID NO: 216 | PRKGGRRRRA WGRRRKRRHR RRRRRRRPRK |
| | RLRLR |
| SEQ ID NO: 217 | PRKGGRRRRA WGRRRKRRKH RRRRRRRPRK |
| | RLRLR |
| SEQ ID NO: 218 | PRKGGRRRRA WGRRRKRRKR RRRRRRRPRK |
| | RLRLR |
| SEQ ID NO: 219 | PRKGGRRRRA WGRRRKRRRH RRRRRRRPRK |
| | RLRLR |
| SEQ ID NO: 220 | PRKGGRRRRA WGRRRKRRRK RRRRRRRPRK |
| | RLRLR |
| SEQ ID NO: 221 | PRKGGRRRRA WGRRRKRRRR RRRRRRRPRK |
| | RLRLR |
| SEQ ID NO: 222 | PRKGGRRRRA WGRRRRRHHH RRRRRRRPRK |
| | RLRLR |
| SEQ ID NO: 223 | PRKGGRRRRA WGRRRRRHHK RRRRRRRPRK |
| | RLRLR |
| SEQ ID NO: 224 | PRKGGRRRRA WGRRRRRHHR RRRRRRRPRK |
| | RLRLR |
| SEQ ID NO: 225 | PRKGGRRRRA WGRRRRRHKH RRRRRRRPRK |
| | RLRLR |
| SEQ ID NO: 226 | PRKGGRRRRA WGRRRRRHKK RRRRRRRPRK |
| | RLRLR |
| SEQ ID NO: 227 | PRKGGRRRRA WGRRRRRHKR RRRRRRRPRK |
| | RLRLR |
| SEQ ID NO: 228 | PRKGGRRRRA WGRRRRRHRH RRRRRRRPRK |
| | RLRLR |
| SEQ ID NO: 229 | PRKGGRRRRA WGRRRRRHRK RRRRRRRPRK |
| | RLRLR |
| SEQ ID NO: 230 | PRKGGRRRRA WGRRRRRHRR RRRRRRRPRK |
| | RLRLR |
| SEQ ID NO: 231 | PRKGGRRRRA WGRRRRRKHH RRRRRRRPRK |
| | RLRLR |
| SEQ ID NO: 232 | PRKGGRRRRA WGRRRRRKHK RRRRRRRPRK |
| | RLRLR |
| SEQ ID NO: 233 | PRKGGRRRRA WGRRRRRKHR RRRRRRRPRK |
| | RLRLR |
| SEQ ID NO: 234 | PRKGGRRRRA WGRRRRRKKH RRRRRRRPRK |
| | RLRLR |
| SEQ ID NO: 235 | PRKGGRRRRA WGRRRRRKKK RRRRRRRPRK |
| | RLRLR |
| SEQ ID NO: 236 | PRKGGRRRRA WGRRRRRKKR RRRRRRRPRK |
| | RLRLR |
| SEQ ID NO: 237 | PRKGGRRRRA WGRRRRRKRH RRRRRRRPRK |
| | RLRLR |
| SEQ ID NO: 238 | PRKGGRRRRA WGRRRRRKRK RRRRRRRPRK |
| | RLRLR |
| SEQ ID NO: 239 | PRKGGRRRRA WGRRRRRKRR RRRRRRRPRK |
| | RLRLR |
| SEQ ID NO: 240 | PRKGGRRRRA WGRRRRRRHH RRRRRRRPRK |
| | RLRLR |
| SEQ ID NO: 241 | PRKGGRRRRA WGRRRRRRHR RRRRRRRPRK |
| | RLRLR |
| SEQ ID NO: 242 | PRKGGRRRRA WGRRRRRRKH RRRRRRRPRK |
| | RLRLR |
| SEQ ID NO: 243 | PRKGGRRRRA WGRRRRRRKK RRRRRRRPRK |
| | RLRLR |
| SEQ ID NO: 244 | PRKGGRRRRA WGRRRRRRKR RRRRRRRPRK |
| | RLRLR |
| SEQ ID NO: 245 | PRKGGRRRRA WGRRRRRRRH RRRRRRRPRK |
| | RLRLR |

Additionally or alternatively, in some embodiments the Senolytic Peptide is any amino acid sequence comprising any peptide having at least 70% identity one of to SEQ ID NO: 1 through SEQ ID NO: 245, or having at least 75% identity one of to SEQ ID NO: 1 through SEQ ID NO: 245, or having at least 80% identity one of to SEQ ID NO: 1 through SEQ ID NO: 245, or having at least 85% identity one of to SEQ ID NO: 1 through SEQ ID NO: 245, or having at least 90% identity one of to SEQ ID NO: 1 through SEQ ID NO: 245, or having at least 95% identity one of to SEQ ID NO: 1 through SEQ ID NO: 245, or having at least 96% identity one of to SEQ ID NO: 1 through SEQ ID NO: 245, or having at least 97% identity one of to SEQ ID NO: 1 through SEQ ID NO: 245, or having at least 98% identity one of to SEQ ID NO: 1 through SEQ ID NO: 245, or having at least 99% identity one of to SEQ ID NO: 1 through SEQ ID NO: 245.

Additionally or alternatively, in some embodiments the Senolytic Peptide is any amino acid sequence comprising any peptide deviating by not more than 10 amino acids from one of to SEQ ID NO: 1 through SEQ ID NO: 245, or deviating by not more than 9 amino acids from one of to SEQ ID NO: 1 through SEQ ID NO: 245, or deviating by not more than 8 amino acids from one of to SEQ ID NO: 1 through SEQ ID NO: 245, or deviating by not more than 7 amino acids from one of to SEQ ID NO: 1 through SEQ ID NO: 245, or deviating by not more than 6 amino acids from one of to SEQ ID NO: 1 through SEQ ID NO: 245, or deviating by not more than 5 amino acids from one of to SEQ ID NO: 1 through SEQ ID NO: 245, or deviating by not more than 4 amino acids from one of to SEQ ID NO: 1 through SEQ ID NO: 245, or deviating by not more than 3 amino acids from one of to SEQ ID NO: 1 through SEQ ID NO: 245, or deviating by not more than 2 amino acids from one of to SEQ ID NO: 1 through SEQ ID NO: 245, or deviating by not more than 1 amino acid from one of to SEQ ID NO: 1 through SEQ ID NO: 245.

In some embodiments, the Senolytic Peptide is any amino acid sequence comprising any peptide one of SEQ ID NO: 1 through SEQ ID NO: 12. Additionally or alternatively, in some embodiments the Senolytic Peptide is any amino acid sequence comprising any peptide having at least 70% identity one of to SEQ ID NO: 1 through SEQ ID NO: 12, or having at least 75% identity one of to SEQ ID NO: 1 through SEQ ID NO: 12, or having at least 80% identity one of to SEQ ID NO: 1 through SEQ ID NO: 12, or having at least 85% identity one of to SEQ ID NO: 1 through SEQ ID NO: 12, or having at least 90% identity one of to SEQ ID NO: 1 through SEQ ID NO: 12, or having at least 95% identity one of to SEQ ID NO: 1 through SEQ ID NO: 12, or having at least 96% identity one of to SEQ ID NO: 1 through SEQ ID NO: 12, or having at least 97% identity one of to SEQ ID NO: 1 through SEQ ID NO: 12, or having at least 98% identity one of to SEQ ID NO: 1 through SEQ ID NO: 12, or having at least 99% identity one of to SEQ ID NO: 1 through SEQ ID NO: 12.

Additionally or alternatively, in some embodiments the Senolytic Peptide is any amino acid sequence comprising any peptide deviating by not more than 10 amino acids from one of to SEQ ID NO: 1 through SEQ ID NO: 12, or deviating by not more than 9 amino acids from one of to SEQ ID NO: 1 through SEQ ID NO: 12, or deviating by not more than 8 amino acids from one of to SEQ ID NO: 1 through SEQ ID NO: 12, or deviating by not more than 7 amino acids from one of to SEQ ID NO: 1 through SEQ ID NO: 12, or deviating by not more than 6 amino acids from one of to SEQ ID NO: 1 through SEQ ID NO: 12, or deviating by not more than 5 amino acids from one of to SEQ ID NO: 1 through SEQ ID NO: 12, or deviating by not more than 4 amino acids from one of to SEQ ID NO: 1 through SEQ ID NO: 12, or deviating by not more than 3 amino acids from one of to SEQ ID NO: 1 through SEQ ID NO: 12, or deviating by not more than 2 amino acids from one of to SEQ ID NO: 1 through SEQ ID NO: 12, or deviating by not more than 1 amino acid from one of to SEQ ID NO: 1 through SEQ ID NO: 12.

In some embodiments, the Senolytic Peptide is any amino acid sequence comprising any peptide one of SEQ ID NO: 1, alternatively, any amino acid sequence comprising any peptide one of SEQ ID NO: 2, alternatively, any amino acid sequence comprising any peptide one of SEQ ID NO: 3, alternatively, any amino acid sequence comprising any peptide one of SEQ ID NO: 4, alternatively, any amino acid sequence comprising any peptide one of SEQ ID NO: 5, alternatively, any amino acid sequence comprising any peptide one of SEQ ID NO: 6, alternatively, any amino acid sequence comprising any peptide one of SEQ ID NO: 7, alternatively, any amino acid sequence comprising any peptide one of SEQ ID NO: 8, alternatively, any amino acid sequence comprising any peptide one of SEQ ID NO: 9, alternatively, any amino acid sequence comprising any peptide one of SEQ ID NO: 10, alternatively, any amino acid sequence comprising any peptide one of SEQ ID NO: 11 alternatively, any amino acid sequence comprising any peptide one of SEQ ID NO: 12.

In some embodiments, the Senolytic Peptide(s) may be characterized with respect to mutations to the native FoxO4 sequence, as shown illustratively in FIG. 18 (N.S., in FIG. 18). For example, the Senolytic Peptide(s) may be represented P-R-K-G-G-1-R-R-2-A-W-G-3-4-5-6-7-8-9-10-11-12-13-14-15-16-17-P-18-K-R-L-19-L-20 (S.P., in FIG. 18), wherein the numbers refer critical positions that exhibit significant interaction with the CR3 domain of FoxO4 (FIG. 12A). Therefore, not intending to be bound by theory, certain mutations to these numbered positions, might enhance the binding of the peptide to FoxO4. In a some embodiments, the mutations present at these positions include one of the amino acids: A, D, E, F, H, I, K, L, N, R, Q, S, Tor Y, for example, so as to enhance the binding affinity of the peptide to FoxO4. Additionally or alternatively, in some embodiments, the Senolytic Peptides include changes (e.g., mutations) at the numbered positions to non-natural amino acids or peptidomimetics can be used to mimic charged amino acids for the numbered positions.

In some embodiments, the Senolytic Peptide is the unique artificial peptide comprising all or substantially all D-amino acids or a mixture of D- and L-amino acids and contains at least 11 mutations to the native FoxO4. Additionally or alternatively, in some embodiments, the Senolytic Peptide(s) is expressed by addition of single or multiple D-amino acids to the N- and/or C-termini, for example, to increase the bioavability of the peptide. For example, in some embodiments, peptides with D-amino acids can mimic L-peptides and modification to D-retroinverso (DRI) forms often change the peptide to a more potent form in vitro and in vivo. These DRI peptide forms have also been shown to be therapeutically effective in clinical trials. As will be recognized by those skilled in the art the one or more of the peptides shown in the SEQUENCE LISTING as SEQ ID NO: 1 through SEQ ID NO: 245, could be expressed by using D-amino acids and in a retroreversed sequence. In some embodiments, the Senolytic Peptide(s) is expressed by using D-amino acids and in a retroreversed sequence.

In some embodiments, the Senolytic Peptide(s) is administered for therapeutic, symptom reducing, or at least partially preventative use against senescence-related pathologies.

In some embodiments, the Senolytic Peptide(s) is suitably administered by fusing to a peptide that facilitates the cell entry from N- or C-terminal. For example, when used as a senolytic agents the Senolytic Peptide(s) need to be enabled to enter into a mammalian cell. Peptides, often called cell-penetrating peptides (CPPs) or protein transduction domains (PTDs), are non-invasive vectors that can efficiently transport various biologically active molecules inside cells. These peptides can transport cargoes ranging from peptides to nanoparticles through a covalent or non-covalent linkage. The transport of the smallest cargo to large 120 kDa proteins was shown to be successful both in vitro and in vivo. Moreover, activable CPPs (ACPPs) have recently reported to perform targeted delivery to cancer cells that overexpress metalloproteinase-2.

In some embodiments of the Senolytic Peptide(s) is expressed using peptidomimetics. For example, peptidomimetics are chemical compounds that may be effective to mimick a natural peptide and to interact with a target so as to exert the same biological effect. Peptidomimetics are often recruited to overcome problems associated with the intrinsic properties of natural peptides such as stability against proteolytic degradation. Peptide bond surrogates and (3-turn dipeptide mimetics among examples of peptidomimetics.

In some edmbodiments, the Senolytic Peptide(s) could be expressed in a cyclic form. For example, in some applications, cyclic peptides may be more effective than their linear counterparts, for example, due to the conformational rigidity. Moreover peptides having a cyclic structure may be relatively resistant to proteolytic cleavage by exopeptidases due to the termini and by endopeptidases as their structure is more rigid than linear peptides.

In some embodiments, the Senolytic Peptide(s) could be expressed as a product of polynucleotides. For example, in some embodiments, polynucleotides that are complementary to at least a portion of a nucleotide sequence encoding the Senolytic Peptide(s) (e.g., a short interfering nucleic acid, an antisense polynucleotide or a peptide nucleic acid) may be prepared using the peptide sequences available in the art to alter gene and/or protein expression. These polynucleotides may specifically bind to or hybridize to certain nucleic acid molecules.

In some embodiments, the polynucleotides may be delivered by a recombinant vector in which the polynucleotide of interest has been incorporated. Additionally or alternaively, in some embodiments, the recombinant viral vector may be a recombinant expression vector into which a polynucleotide sequence that encodes the designed peptides has been incorporated. The recombinant vector or the recombinant expression vector may be a viral recombinant vector or a viral recombinant expression vector. As will be recognized by those skilled in the art the Senolytic Peptide(s) could be expressed via a vector.

In some embodiments, the Senolytic Peptide(s) may comprise one or more unnatural or unusual amino acids, for example, that are not naturally encoded amino acids. These may be non-proteinogenic amino acids which can occur naturally in plant or bacteria post-translationally or are chemically synthesized as pharmacological motifs. Unnatural amino acids are widely used in combinatorial libraries or as chiral building blocks. These modified amino acids often contribute to special biological activity, and are often incorporated into therapeutic peptidomimetic ligands to enhance peptide pharmacological activity and potency. Unusual or unnatural amino acid modified peptides can improve selectivity, receptor binding, modify activity (agonists and antagonists), increase in vivo half-life, enhance transport across cell membranes. As will be recognized by those skilled in the art, the disclosed CPPs are expressed by a wide range of unnatural, non-standard amino acid peptide modifications. In some embodiments, modifications to CPPs include incorporation of unnatural/unusual amino acids into the sequence using D-amino acids, L-amino acids, N-methyl amino acids, homo amino acids, alpha-methyl amino acids, beta (homo) amino acids, gamma amino acids, helix/turn stabilizing compound or backbone modification peptoides. Additionally or alternatively, in some embodiments, CPP sequence(s) are modified by insertion of natural occurring unusual amino acids such as citrulline (Cit), hydroxyproline (Hyp), beta-alanine, ornithine (Orn), norleucine (Nle), 3-nitrotyrosine, pyroglutamic acid (Pyr), nitroarginine, Alanine Modified Amino Acid Analogs, Alicyclic Amino Acids Analogs, Aromatic Amino Acids Analogs, Aspartic Acid Analogs, Beta-Amino Acids Analogs, Cysteine Amino Acid Analogs, DAB (2,4-Diaminobutyric Acid) Analogs, DAP (2,3-Diaminopropionic Acid) Analogs, Depsipeptides, Glutamic Acid Amino Acid Analogs, Glutamine Amino Acid Analogs, Glycine Amino Acid Analogs, Heterocyclic Amino Acid Analogs, Histidine Amino Acid Analogs, Homo-Amino Acid Analogs, Isoleucine Amino Acid Analogs, Leucine Amino Acid Analogs, Linear Core Amino Acid Analogs, Lysine Amino Acid Analogs, Methionine Amino Acid Analogs, N-Methyl Amino Acids Analogs, Norleucine Amino Acid Analogs, Norvaline Amino Acid Analogs, Ornithine Amino Acid Analogs, Statine Amino Acid Analogs, Penicillamine Amino Acid Analogs, PEGylated Amino Acids, Phenylalanine Amino Acid Analogs, Phenylglycine Amino Acid Analogs, Proline Amino Acid Analogs, Pyroglutamine Amino Acid Analogs, Serine Amino Acid Analogs, Threonine Amino Acid Analogs, Tryptophan Amino Acid Analogs, Tyrosine Amino Acid Analogs, Valine Amino Acid Analogs, Post-Translational Modification (PTM) Modified Amino Acids, Deiminated Arginines (Citrulline), DL-Citrulline, L-Citrulline, Methylated Aminod Acids, Lysine(Me), Lysine(Me2), Lysine(Me3), Arginine(Me), Arginine(Me)2 asymmetrical, Arginine(Me)2 symmetrical, Phosphorylated Amino Acids, Phosphoserine, phosphothreonine, phosphotyrosine.

In various embodiments, the Senolytic Peptide(s), for example, one or more of SEQ ID NO: 1 through SEQ ID NO: 245 as shown in the SEQUENCE LISTING or derivatives thereof, for example, peptides containing not more of 30% mutations with respect to these sequences, may also interfere with CR3 domain of FoxO4 thereby interfering with SCAP. Furthermore, the Senolytic Peptide(s) that are formed by combining or shuffling some part of one or more peptide sequences that is at least 6 amino acid in length shall carry the CR3 domain binding affinity thereby interfering with FoxO4.

In some embodiments, methods for selectively inducing apoptosis in (e.g., killing) senescent cells in a subject who has a senescence-associated disease or disorder may generally comprise administering one or more of the Senolytic Peptide(s) to the subject in need thereof, for example, according to one or more of the administration methods described herein. For example, in some embodiments, the method may comprise causing an artificial peptide comprising an amino acid sequence having at least 90% identify to any one of SEQ ID NO: 1 through SEQ ID NO: 245 to interfere with the CR3 domain of Forkhead box protein O4 (FoxO4) of the senescent cell.

The proportion of senescent cells in a tissue of a mammal varies with the biological age and could vary substantially depending on the cohort that mammal subject belongs to. Moreover, the proportion of senescent cells present may further vary with the type of the tissue in a given subject. These variations may create a challenge in specifying a dose for the senolytic agent for a rejuvenation therapy. Additional complexities in specifying a dose arise when said senescent cell proportion is above a certain threshold and therefore accelerated apoptosis could result in frailty. Fortunately, unlike for cancer interventions, complete elimination of senescent cells may not be necessary for achieving beneficial effects.

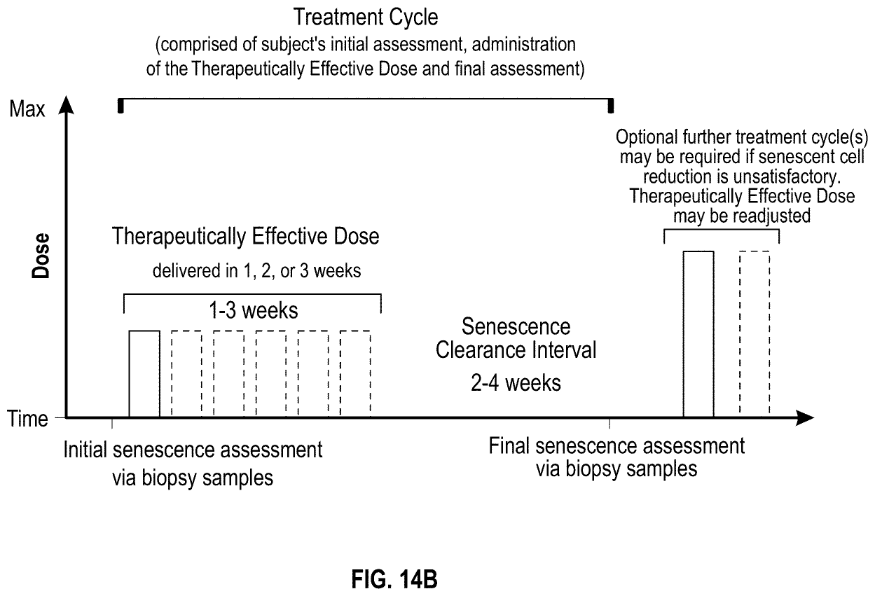

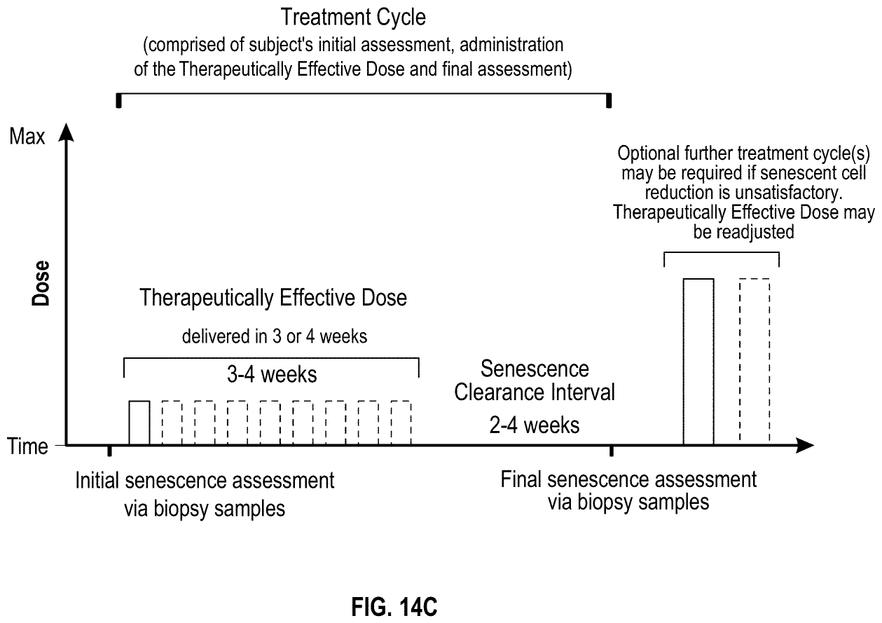

In various embodiments, the Senolytic Peptides may be administered via any suitable methodolgy. For example, disclosed herein three different treatment regimes, namely an Impulse Regime, a Sustained Regime, and Gentle Regime (shown illustratively in FIGS. 14A, 14B, and 14C) via which the Senolytic Peptide(s) may be administered.

In some embodiments, a method is provided for treating a senescence-associated disease or disorder comprising administering to a subject in need thereof a Therapeutically Effective Dose of the Senolytic Peptide(s), for example, the Senolytic Peptide(s) may be administered intermittently in one or multiple treatment cycles. Each treatment cycle may extend over 1 or 2 or 3 days. Each administration may include equivalent doses adjusted so as to cumulatively reach the Therapeutically Effective Dose at the end of each treatment cycle. In some embodiments, the Therapeutically Effective Dose is administered equivalently in a single day or intermittently in two consecutive days or in three consecutive days or 2 or 3 administration in alternate days. This regime of administration of the Senolytic Peptides is referred to herein as the Impulse Regime (FIG. 14A). In the Impulse Regime, the Therapeutically Effective Dose is achieved through one or two subsequent administration(s). In some embodiments, after the first cycle, there may be a two week-four week senescence clearance interval, for example, allowing a period of time effective for a decrease in senescent cells. In some embodiments, the subject is evaluated, both before the Impulse Shock Regime and after Senescence Clearance Interval period, by one skilled in the art to determine levels of various SASP markers for determination of the Therapeutically Effective Dose and Follow-up Treatment, respectively.

Additionally or alternatively, in some embodiments, the Therapeutically Effective Dose is achieved through a single or multiple administrations in a period of 1-3 weeks. The quantity of the Senolytic Peptide for each administration is equivalent and adjusted to cumulatively reach the Therapeutically Effective Dose at the end of the treatment. This regime of administration of the Senolytic Peptides is refered to as the Sustained Regime (FIG. 14B). The Therapeutically Effective Dose administered in the Sustained Regime may be higher than the Therapeutically Effective Dose as would be administered in the Impulse Regime.

Additionally or alternatively, in some embodiments The Therapeutically Effective Dose is administered intermittently in one or multiple treatment cycles wherein each treatment cycle comprised of 1 or 2 or 3 or 4 or 5 or 6 administration days equally distributed in 1-3 weeks where each administration is in equivalent doses adjusted to cumulatively reach the Therapeutically Effective Dose at the end of each treatment cycle.

In some embodiments, after the first cycle, there is a two or three weeks senescence clearance interval allowing a period of time effective for a decrease in senescent cells. In some embodiments, the subject may be evaluated, both before the Sustained Regime and after Senescence Clearance Interval period, by one skilled in the art based on the levels of various SASP markers for determination of the Therapeutically Effective Dose and Follow-up Treatment, respectively.

In some embodiments, the Therapeutically Effective Dose is achieved through single or multiple administration cycles in the period of 3-4 weeks. This regime of administration of the Senolytic Peptides is referred to herein as the Gentle Regime (FIG. 14C). Both the Therapeutically Effective Dose and the quantity of the Senolytic Peptide for a single administration in the Gentle Regime may be lower than those in the Impulse Regime and Sustained Regime. The Therapeutically Effective Dose may be administered intermittently in one or multiple treatment cycles. Each treatment cycle may be comprised of 1 or 2 or 3 or 4 or 5 or 6 or 7 or 8 or 9 administration days equally distributed in 3-4 weeks. Each administration may be in an equivalent dose adjusted so as to cumulatively reach the Therapeutically Effective Dose at the end of each treatment cycle.

In some embodiments, after the first treatment cycle, there is a two or three weeks Senescence Clearance Interval allowing a period of time for the treatment becoming effective with gradual decrease in senescent cells. In some embodiments, the subject may be evaluated both before the Gentle Regime and after Senescence Clearance Interval period, by those skilled in the art based on the levels of various SASP markers for determination of the Therapeutically Effective Dose and Follow-up Treatment, respectively.

In some embodiments, the Therapeutically Effective Dose amount is delivered by intraperitoneal injection (IP) or intravenous injection (IV). In some embodiments Therapeutically Effective Dose amount is further adjusted depending on whether it is delivered by IP or IV.

In various embodiments, the treatment regimes is defined with respect to the amount of Senolytic Peptide, both in terms of the quantity administered and the frequency sufficient for the desired therapeutic effect, that is, the Therapeutically Effective Dose. A therapeutic effect entails, to some extent, some effect with respect to one or more of the symptoms of the disease, and includes curing a disease. “Curing” means that the symptoms of active disease are eliminated. However, certain long-term or permanent effects of the disease may exist even after a cure is obtained (such as where extensive tissue damage is present). The Therapeutically Effective Dose is typically one which is sufficient to achieve the desired effect and may vary according to the nature and severity of the disease condition, and the potency of the particular Senolytic Peptide administered, its purity, and its composition (e.g. 90%, or 95% or 98% etc.). In some embodiments, different doses may be employed for embodiments where the Senolytic Peptide(s) is administered for preventive use rather than for treatment of an active disease. In some embodiments Therapeutically Effective Dose for the Senolytic Peptides is as described in Schedule 1.

As discussed herein below, examples 1-5 indicate that the Senolytic Peptide(s) can safely be administered at doses of 5 mg, 10 mg, and 15 mg. More particularly, Example 4 indicates that the Senolytic Peptide(s) is non-toxic at levels upto 100 mg/kg. Due to the molecular similarity of the various Senolytic Peptides, particularly, SEQ ID NO: 1 through SEQ ID NO: 11 and peptides having at least 90% identify thereto, atomistic structures are similar and therefore they exert their senolytic effects in a similar way. In some embodiments, the Therapeutically Effective Dose amount can further depend upon the patient's height, weight, sex, age and medical history.

In some embodiments, treatment methods, such as according to the Impulse Regime, Sustained Regime, or Gentle Regime, may include monitoring the population of the senescent cells on convenient biological samples taken from the subject at the beginning and 2 or 4 weeks after the treatment to determine the effectiveness of the therapy and or whether a second or a third treatment course is required. In some embodiments, an individualized treatment course may be implemented and may include monitoring of senescent cell population at the beginning of a therapy and a certain period after each treatment course and adjusting the treatment course or the dose or the treatment regime. Additionally or alternatively, in some embodiments the administration of a Therapeutically Effective Dose may depend upon the subject's state of health and subject's response to the treatment throughout the period of the therapy. In some embodiments, the biological sample shall be the skin biopsy specimens obtained from skin tissue of the subject is collected with minimally invasive methods. In some embodiments, the detection of the senescent cells may be achieved using senescence associated markers

Although senescent cells diverge from other quiescent and terminally differentiated cells, they do not display a unique phenotype but a variety of phenotypes which define the senescent state. Hallmarks of the senescent cells in this state include permanent and irreversible growth arrest; increase in cell size; expression of senescence-associated beta-galactosidase (SA-β-Gal) enzyme resulting in increased lysosomal content; expression of a tumor suppressor, p16^{INK4a}. Two biomarkers have been extensively used for identification of senescent cells:
SA-β-Gal: Enzyme activity assayed at pH 6 using X-Gal as the substrate. p16^{INK4a}: Monitoring the expression levels of p16^{INK4a} protein which is a CDK4/6 inhibitor and is involved in maintenance of growth arrest.

In some embodiments, the Senolytic Peptide(s) do not have to be continuously present to exert an intended effect. For example, brief disruption of pro-survival pathways is adequate to kill senescent cells. Thus the Senolytic Peptide(s) can be effective as Senolytic Peptides when administered intermittently.

It has already been reported that other known senolytic agents such as the tyrosine kinase inhibitor (D) and the flavonoid, quercetin (Q), were shown to induce apoptosis in senescent cells. Intermittent administration of D+Q alleviated frailty, neurological dysfunction, osteoporosis, and vertebral disk degeneration related to loss of glycosaminoglycans on an accelerated aging-like state. Furthermore, in mice with impaired mobility due to radiation of one of their legs 3 months previously, tread-mill endurance improved within 4 days after completing a single course of D+Q. Said improvement persisted for at least 7 months. D+Q has an elimination half-life of a few hours. These outcomes following intermittent or single courses of agents with short elimination half-lives are consistent with the long-lasting type of effect expected from reducing senescent cell abundance, as opposed to what would be expected if D+Q had to be continuously present to suppress or activate cellular processes by occupying a receptor or acting on an enzyme.

Thus, intermittent rather than continuous treatment with senolytics may be effective in alleviating senescence-related diseases or disorders, allowing these agents to be administered during periods of good health and potentially decreasing risk of side-effects. In all of the shock dose regimes, there will be an optional follow-up treatment wherein the Therapeutically Effective Dose can be increased or decreased. In between the initial treatment and follow-up treatment there will be a non-treatment interval, wherein the non-treatment course duration could vary depending on the subject's state of health and/or the subject's response to the treatment which can be monitored by the said detection of senescent cells in the subject throughout the period of the therapy.

In some embodiments, at least one Senolytic Peptide may be administered in a Therapeutically Effective Dose with at least one or more other available senolytic agent which together act additively or synergistically to selectively kill senescent cells. For example, in some embodiments, a senolytic drug administration method is used for dose optimization of the senolytic agents of any kind including but not limited to Dasatinib, Quercetin, Navitoclax, Piperlongumin, Fisetin, BCL-XL inhibitors A1331852 and A1155463, FOXO4-related peptide or combinations of these.

The effectiveness of the Senolytic Peptide treatment can be determined by a person skilled in the medical and clinical arts by employing combination of diagnostic methods, including physical examination, assessment and monitoring of clinical symptoms, and performance of analytical tests and methods described herein, may be used for monitoring the health status of the subject, in addition to the senescent cell monitoring described herein for the treatment regimes. For example in one embodiment the effectiveness of the Senolytic Peptides when treating a subject for pulmonary diseases and disorders Pulmonary Function Tests (https://www.nhlbi.nih.gov/health/health-topics/topics/lft) can be conducted. Pulmonary function tests, or PFTs, measure how well subject's lungs work. They include tests that measure lung size and air flow, such as spirometry and lung volume tests. Other tests measure how well gases such as oxygen get in and out of subject's blood. These tests include pulse oximetry and arterial blood gas tests. Another pulmonary function test, called fractional exhaled nitric oxide (FeNO), measures nitric oxide, which is a marker for inflammation in the lungs. The subject may have one or more of these tests to diagnose lung and airway diseases, compare subject's lung function to expected levels of function, monitor if your disease is stable or worsening, and see if said senolytic treatment is beneficial.

It should be noted that FoxO4 is expressed in a tissues during fast growth or regeneration. As such, in some embodiments a senolytic therapy using the Senolytic Peptide(s) may exclude such cohorts.

In some embodiments using the Senolytic Peptide(s) on a subject, senescent cell population in the Biological skin sample of the subject undergoing treatment will be monitored before, and after a treatment course. In some embodiments, monitoring is performed during a treatment course and/or between the treatment courses or cycles.

Method of Use of the Senolytic Peptides—Schedule 1

In some embodiments, the senolytic agent comprises any one of the Senolytic Peptides and in some embodiments, the senolytic agent is administered in a treatment window comprising 11 to 28 days.

For example, in some embodiments, the Senolytic agent is administered daily or alternating days for 14 days followed by minimum 14 days off.

In some embodiments, the senolytic agent is administered daily for 13 days followed by minimum 14 days off.

In some embodiments, the senolytic agent is administered daily or alternating days for 12 days followed by minimum 14 days off.

In some embodiments, the senolytic agent is administered daily for 11 days followed by minimum 14 days off.

In some embodiments, the senolytic agent is administered daily or alternating days for 10 days followed by minimum 14 days off.

In some embodiments, the senolytic agent is administered daily for 9 days followed by minimum 14 days off.

In some embodiments, the senolytic agent is administered daily or alternating days for 8 days followed by minimum 12 days off.

In some embodiments, the senolytic agent is administered daily for 7 days followed by minimum 12 days off.

In some embodiments, the senolytic agent is administered daily or alternating days for 6 days followed by minimum 12 days off.

In some embodiments, the senolytic agent is administered daily for 5 days followed by minimum 12 days off.

In some embodiments, the senolytic agent is administered daily or alternating days for 4 days followed by minimum 12 days off.

In some embodiments, the senolytic agent is administered daily for 3 days followed by minimum 10 days off.

In some embodiments, the senolytic agent is administered daily for 2 days followed by minimum 10 days off.

In some embodiments, the senolytic agent is administered for 1 day followed by minimum 10 days off.

In some embodiments, the senolytic agent is administered daily for 14 days in a dose of about 0.5 mg/kg to 20 mg/kg.

In some embodiments, the senolytic agent is administered daily for 14 days in a dose of about 0.5 mg/kg to 15 mg/kg.

In some embodiments, the senolytic agent is administered daily for 14 days in a dose of about 0.5 mg/kg to 10 mg/kg.

In some embodiments, the senolytic agent is administered daily for 14 days in a dose of about 0.5 mg/kg to 5 mg/kg.

In some embodiments, the senolytic agent is administered daily for 14 days in a dose of about 0.5 mg/kg to 3 mg/kg.

In some embodiments, the senolytic agent is administered daily for 14 days in a dose of about 1400 mg per day.

In some embodiments, the senolytic agent is administered daily for 14 days in a dose of about 1000 mg.

In some embodiments, the senolytic agent is administered daily for 14 days in a dose of about 700 mg.

In some embodiments, the senolytic agent is administered daily for 14 days in a dose of about 200 mg.

In some embodiments, the senolytic agent is administered daily for 14 days in a dose of about 35 mg.

In some embodiments, the senolytic agent is administered daily for 7 days in a dose of about 35 mg to 1400 mg.

In some embodiments, the senolytic agent is administered daily for 7 days in a dose of about 35 mg to 1000 mg.

In some embodiments, the senolytic agent is administered daily for 7 days in a dose of about 35 mg to 700 mg.

In some embodiments, the senolytic agent is administered daily for 7 days in a dose of about 35 mg to 200 mg.

In some embodiments, the senolytic agent is administered daily for 7 days in a dose of about 1400 mg.

In some embodiments, the senolytic agent is administered daily for 7 days in a dose of about 1000 mg.

In some embodiments, the senolytic agent is administered daily for 7 days in a dose of about 700 mg.

In some embodiments, the senolytic agent is administered daily for 7 days in a dose of about 200 mg.

In some embodiments, the senolytic agent is administered daily for 7 days in a dose of about 35 mg.

In some embodiments, the senolytic agent is administered in one of the above doses daily for 1, 2, 3, 4, 5, or 6 days or 8, 9, 10, 11, 12, 13 days. Additionally or alternatively, in sotme embodiments, the senolytic agent is administered in one of the above doses are administered alternative daily for 2, 4, or 6 days or 8, 10, 12, 14 days.

In some embodiments the doses described in this Schedule 1 shall be a Therapeutically Sufficient Dose for the administration of any one of the Senolytic agents.

In some embodiments the regiment of administration in this Schedule 1 shall be the method of administration of any one of the Senolytic agents.

In some emdodiments Senolytic Peptide(s) is administered as described in this Schedule 1 together with another senolytic agent including but not limited to Navitoclax (ABT-263), Fisetin, A133185240, A115546340, Quercetin, Dasatinib, Piperlongumine, 17-AAG (tanespimycin), Geldanamycin 17-DMAG (alvespimycin), Famotidine, Deferoxamine, Mitoxantrane, Lapatinib, Neratinib with therapeutically sufficient doses for each of these snolytical agents but strictly in the regiments as decribed herein this Schedule 1.

The effectiveness of the Senolytic Peptide treatment can be determined by a person skilled in the medical and clinical arts by employing combination of diagnostic methods, including physical examination, assessment and monitoring of clinical symptoms, and performance of analytical tests and methods described herein, may be used for monitoring the health status of the subject, in addition to the monitoring described herein for the treatment regimes.

Medical Therapies Uing the Senolytic Peptides

The effectiveness of the Senolytic Peptide(s) with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, subject self-assessment, assessment and monitoring of clinical symptoms, performance of analytical tests and methods, including clinical laboratory tests, physical tests, and exploratory surgery, for example, may be used for monitoring the health status of the subject and the effectiveness of the Senolytic Peptide. The effects of the methods of treatment described herein can be analyzed using techniques known in the art, such as comparing symptoms of subjects suffering from or at risk of a particular disease or disorder that have received the composition comprising the Senolytic Peptide with those of subjects who were not treated with the Senolytic Peptide or who received a placebo treatment.

A subject in need of treatment with the Senolytic Peptide(s) as described herein may be a human or may be a non-human primate or other animal (i.e., veterinary use) who has developed symptoms of a senescence cell-associated disease or disorder or who is at risk for developing a senescence cell-associated disease or disorder. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, elephants, bears and other domestic, farm, and zoo animals.

In some embodiments, administration of the Senolytic Peptide(s) described herein can prolong survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving the Senolytic Peptide would provide clinical benefit to delay development or reduce severity of a disease, including an age-related disease or disorder.

In some embodiments, use of the Senolytic Peptides may be restricted during wound healing (including pre- or post-operations). When a wound is present, senescent cells may be induced around the wound. Senescent cells make growth factors that are required for wound healing. However, this innate mechanism is not disturbed unless the Senolytic Peptide(s) is administered at the time of the wound healing.

Method of Use of the Senolytic Peptides in Senescence-Associated Diseases and Disorders

Cellular senescence is a cell fate that involves essentially irreversible replicative arrest, apoptosis resistance, and frequently increased protein synthesis, metabolic shifts with increased glycolysis, decreased fatty acid oxidation, increased reactive oxygen species generation, and acquisition of a senescence-associated secretory phenotype (SASP).

Methods are provided herein for treating conditions, diseases or disorders related to, associated with, or caused by cellular senescence in a subject in need thereof. In some embodiments the method of use for the treatment of the above stated senescence-associated diseases and disorders comprises administration of a Therapeutically Effective Dose of the Senolytic Peptides, for example, in one of the three different treatment regimes namely Impulse Regime, Sustained Regime, and Gentle Regime. In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the dose regime is applied subject to the decision of the doctor overseeing the therapy.

Method of Use of the Senolytic Peptide(s) for treatment of general Age-Related Diseases and Disorders

In some embodiments, the Senolytic Peptide(s) inhibits senescence of adult stem cells or inhibits accumulation, kills, or otherwise facilitates removal of adult stem cells that have become senescent. Therefore, the Senolytic Peptides may also be useful for treating or preventing of an age-related disease or disorder that occurs as part of the natural aging process or that occurs when the subject is exposed to a senescence inducing agent or factor (e.g., irradiation, chemotherapy, smoking tobacco, high-fat/high sugar diet, other environmental factors).

In some embodiments, frailty as an aging-associated decline may be treated or prevented (i.e., the likelihood of occurrence of is reduced) by administering the Senolytic Peptide. The effectiveness of the senolytic therapy can be measured by monitoring the fraility index of the patient.

In some embodiments, the age related disease or disorder is scoliosis. Effectiveness of the senolytic therapy are measured by, inter alia, physical examination of spine, ribs, hips and shoulders and/or X-RAY, CT and/or MRI to determine bone curvature.

In some embodiments involving treatment of age-related diseases and disorders the Sustained Regime is most suitable (FIG. 14B). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the impulse Regime or Gentle Regime (FIG. 14A-C) or the method of administration described in Schedule 1 are applied subject to the decision of the doctor overseeing the therapy.

The effectiveness of a method of treatment described herein may be manifested by reducing the number of symptoms of an age-related disorder or age-sensitive trait associated with a senescence-inducing stimulus, decreasing the severity of one or more symptoms, or delaying the progression of an age-related disorder or age-sensitive trait associated with a senescence-inducing stimulus. In some embodiments, preventing an age-related disorder or age-sensitive trait associated with a senescence-inducing stimulus refers to preventing (i.e., reducing the likelihood of occurrence) or delaying onset of an age-related disorder or age-sensitive trait associated with a senescence-inducing stimulus, or reoccurrence of one or more age-related disorder or age-sensitive trait associated with a senescence-inducing stimulus.

Method Use of the Senolytic Peptide(s) in Tissue Rejuvenation

It has been reported in the reference that a senolytic agent could influence the health span of mice in which senescence and the concomitant loss of tissue homeostasis were allowed to develop spontaneously as a consequence of aging. The results of in vivo mouse experiments indicate that a senolytic agent can reduce cellular senescence and counteract hair loss (objectively measured as the fur density) and general frailty (objectively measured by increased running wheel activity) in mice.

Tissues contain high levels of senescent cells, which due to chronic SASP secretion, would inflict permanent reprogramming on their neighboring cells. Senescence was recently shown to trigger tissue reprogramming in vivo, leading to Nanog-positive cells in the vicinity of areas of senescence. Senescent cells might thus trigger reprogramming of neighboring cells into more pluripotent cells. However, since the release of SASP factors (such as IL6) is continuous, they would effectively make this change permanent and keep their neighboring recipient cells locked in this stem like state. If the number of senescent cells are reduced to that of relatively young tissues with few senescent cells then a transient SASP response, causing temporary cell reprogramming and subsequent proliferation/differentiation responses would be able to replenish damaged and lost cells. In some embodiments the Senolytic Peptide(s) is applied for tissue rejuvenation therapy of a scalp tissue or an osteoarthritic joint or a pulmonary tissue or a renal tissue. In some embodiments senolytic peptide is used for scalp treatment and hair regeneration by applying the senolytic tissue externally. In some embodiments the method of delivery of senolytic peptides in treatment of senescence-associated diseases and disorders are detailed herein this document.

In some embodiments wherein the treatment aims tissue rejuvenation the Impulse Regime most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) are applied subject to the decision of the doctor overseeing the therapy.

Method Use of the Senolytic Peptide(s) for Treatment of Senescence-Associated Dermatological Disease or Disorders

Senescence-associated diseases or disorders treatable by administering the Senolytic Peptide described herein include dermatological diseases or disorders. Several dermatological diseases or disorder are associated with the accumulation of senescent cells. In some embodiments, said dermatological diseases and disorders are psoriasis, vitiligo and eczema. Among them, vitiligo is an acquired disorder characterized by depigmentation. In addition to genetic susceptibility and autoimmunity, oxidative stress and underlying premature melanocyte senescence are considered to be key factors in vitiligo progression. Melanocytes from non-lesional vitiligo lesions were shown to exhibit a pre-senescent phenotype in vitro.

Therefore, in some embodiments, Senolytic Peptide(s) is suitable to cure or treat dermatological diseases and disorders by killing the senescent cells.

In some embodiments involving treatment of dermatological diseases and disorders the Impulse Regime is most suitable (FIG. 14A). In various embodiments the Therapeutically Effective Dose and/or the duration of treatment of above stated regimes could be altered depending on the subject's state of health and/or the subject's response to the treatment which can be monitored. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) are applied subject to the decision of the doctor overseeing the therapy. In certain appropriate senescence-associated dermatological diseases and disorders the method of application of the senolytic peptides could be external on the surface of the body such that skin cell penetration of said peptides is achieved as described in detailed herein this document.

### Method Use of the Senolytic Peptide(s) for Treatment of Inflammatory and Autoimmune Diseases and Disorders

Chronic inflammation is a major factor in wide range of disease associated with old age. Recently a global clinical trial of 10,000 subjects who had previous heart attacks of an anti-inflammatory drug which targeted a portion of the inflammatory pathway—focusing specifically on interleukin- lbeta (IL-1(3), a cell-signaling protein—showed that it reduced their risk of further heart attacks or strokes. The drug prevents these cells from going into overdrive, but presumably leaves the remaining immune system intact.

The senescence-associated secretory phenotype (SASP) comprises a range of different proteins, including several proteins known to play a role in aging and age-related diseases, including chemokines such as CCL2 and CLL11 and prominent interleukins such as IL-1, IL-6 and IL-12. When above a certain threshold, such factors can significantly impair tissue function and impair functioning of neighboring cells. As such, senescent cells are thought to be major contributors of inflammation; a theory stating that low, but chronic, levels of inflammation are drivers of age-related decline.

In some embodiments, chronic inflammatory diseases involve Rheumatoid arthritis targeting the joints. Individuals with Rheumatoid arthritis exhibit accelerated immunosenescence possibly as a result of inflammatory mechanisms.

In some embodiments, chronic inflammatory diseases involve osteoarthritis characterized by progressive tissue remodeling and loss of joint function and paralleled by increased age. It is the most prevalent disease of the synovial joints. During osteoarthritis, levels of various senescence markers increase in chondrocytes with SASP profiles similar to classical senescent cells which in turn supports the hypothesis that senescence of cells within joint tissues may play a pathological role in the causation of osteoarthritis. Therefore, the Senolytic Peptide(s) is suitable to cure or manage chronic inflammation by substantially stopping SASP by killing the senescent cells.

In some embodiments, the Senolytic Peptide(s) is effective in the treatment of osteoarthritis. During the senolytic treatment, the osteoarthritis disease parameters which are measured include, inter alia, joint pain, redness, stiffness and/or swelling and joint motion range, X-RAY and/or MRI for bone spurs, blood tests and joint fluid analyses to rule out other causes.

In some embodiments, the Senolytic Peptide(s) is effective in the treatment of kyphosis. During the senolytic treatment, the kyphosis disease parameters which are measured include, inter alia, measurement of spine curvature by X-RAY, CT and/or MRI.

In some embodiments involving treatment of inflammatory or autoimmune diseases and disorders Gentle Regime is most suitable (FIG. 14C). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Impulse Regime or Sustained Regimes (FIG. 14A-C) a or the method of administration described in Schedule 1 reapplied subject to the decision of the doctor overseeing the therapy.

Method Use of the Senolytic Peptide(s) for treatment of Cardiovascular Diseases and Disorders

In one embodiment, the senescence-associated disease or disorder treated by the methods described herein is a cardiovascular disease. In some embodiments, cardiovascular disease is caused by atherosclerosis and is the primary cause of mortality in the developed countries.

There is a growing body of evidence for inflammation as a key process in atherosclerosis into clinical and public health practice. Atherosclerosis is a disease of major arteries in which high levels of low-density lipoprotein bearing oxidative modifications accumulate in vessel walls, attracting phagocytic immune cells to form plaques. Telomere shortening and oxidative stress caused by smooth-muscle proliferation and declining levels of endothelial nitric oxide synthase during plaque formation and expansion cause senescence induction. Human and mouse atheromas have been reported to exhibit senescent vascular smooth muscle and endothelial cells. Basic science and epidemiological studies have developed an impressive case that atherogenesis is essentially an inflammatory response to a variety of risk factors and the consequences of this response lead to the development of acute coronary syndrome. These findings raise the possibility of multi-step involvement of senescent cells in atherogenesis. Therefore, in some embodiments, the Senolytic Peptide(s) is suitable to slow down the progression of cardiovascular disease by reducing the chronic inflammation in the body substantially by stopping SASP.

During the Senolytic Peptide treatment of (cardio)vascular disease, (cardio)vascular disease parameters which are measured include, inter alia, cardiac ejection fraction, blood vessel stiffness and blood pressure.

Senolytic therapy is effective in treatment of atherosclerosis. During the Senolytic Peptide treatment atherosclerosis disease parameters which are measured include blood tests including measurements of cholesterol, glucose, electrocardiogram, angiography, computerised tomography scan and/or ophthalmoscopy.

The effectiveness of one or more Senolytic Peptides for treating or preventing (i.e., reducing or decreasing the likelihood of developing or occurrence of) a cardiovascular disease (e.g., atherosclerosis) can readily be determined by a person skilled in the medical and clinical arts. Health status of the subject may be monitored by one or any combination of diagnostic methods, including but not limited to physical examination, assessment and monitoring of clinical symptoms, and performance of analytical tests and methods described herein and practiced in the art (e.g., angiography, electrocardiography, stress test, non-stress test). The effects of the treatment of the Senolytic Peptide can be analyzed using techniques known in the art, such as comparing symptoms of subjects suffering from or at risk of cardiovascular disease that have received the treatment with those of subjects without such a treatment or with placebo treatment.

In some embodiments involving treatment of cardiovascular diseases and disorders the Impulse Regime is the most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) or the method of administration described in Schedule 1 are applied subject to the decision of the doctor overseeing the therapy.

Method Use of the Senolytic Peptide(s) for Treatment of Pulmonary Diseases and Disorders

In one embodiment, the senescence-associated disease or disorder treated by the methods described herein is a Pulmonary Diseases and Disorders.

In some embodiments, Pulmonary Disease and Disorders treated by the methods decribed herein include Chronic obstructive pulmonary disease (COPD)/emphysema characterized by lung inflammation induced by accelerated lung aging involving inflammatory mediators such as tumor necrosis factor alpha, interleukin-1, interleukin-6, reactive oxygen species and proteases. Mechanisms involved in COPD include telomere shortening, cellular senescence, activation of PI3 kinase-mTOR signaling, impaired autophagy, mitochondrial dysfunction, stem cell exhaustion, epigenetic changes, abnormal microRNA profiles, immunosenescence, and a low-grade chronic inflammation.

In some embodiments, Pulmonary Disease and Disorders treated by the methods decribed herein include Idiophatic Pulmonary Fibrosis (IPF). IPF is the most common and severe idiopathic interstitial pneumonia. In familial interstitial pneumonia, the telomerase complex is affected by the genetic lesions present eventually leading to telomere shortening in both leukocytes and pulmonary tissue which is also observed in sporadic IPF. Pathology of IPF points out to a mechanism with the involvement of cellular senescence in disease progression.

In both COPD and IPF, premature cellular senescence likely affects distinct progenitors cells (mesenchymal stem cells in COPD, alveolar epithelial precursors in IPF), leading to stem cell exhaustion.

Therefore, the Senolytic Peptide(s) is suitable for treatment of premature senescence involved in Pulmonary Diseases or Disorders by removing the senescent cells in controlled and safe manner.

In one embodiment, methods are provided for treating ore preventing (i.e., reducing the likelihood of occurrence of) a senescence-associated disease or disorder that is a pulmonary disease or disorder by killing senescent cells associated with the disease or disorder particularly the senescence of pulmonary artery-smooth muscle cells in a subject who has the disease or disorder by administering the Senolytic Peptide.

In some embodiments, the Senolytic Peptide(s) is effective in the treatment of lung emphysema. During the senolytic therapy, the lung emphysema disease parameters which are measured include, inter alia, breathlessness, chest size, decreased breath sounds through the stethoscope, fingertip shape, style of breathing, hypoxemia, hypercaria, cyanosis, malnutrition. Lung volume, lung ejection capacity, dead volume in the lungs, airflow changes after bronchodilator medication, chest X-RAY and CT scan of the chest and red blood cell counts.

In some embodiments, the Senolytic Peptide(s) is effective in the treatment of COPD. During the senolytic therapy of the COPD disease parameters which are measured include, inter alia, spirometry and lung functional tests as described for lung emphysema, including breathlessness, chest size, decreased breath sounds through the stethoscope, fingertip shape, style of breathing, hypoxemia, hypercaria, cyanosis, malnutrition, lung volume, lung ejection capacity, dead volume in the lungs, airflow changes after bronchodilator medication, chest X-RAY and CT scan of the chest and red blood cell counts.

In some embodiments involving treatment of Pulmonary Diseases and Disorders seneloytic peptide delivery is done via a nebuliser. When used with a nebuliser, the Senolytic Peptide (e.g., which is soluble) may be mixed with saline solutions and the dose specifications and administration timetable are as described in the Schedule 1. In other embodiments Impulse Regime is most suitable (FIG. 14A). In various embodiments the Therapeutically Effective Dose and/or the duration of treatment of above stated regimes could be altered depending on the subject's state of health and/or the subject's response to the treatment. During the therapy, a subjects progress can be monitored appropriate measurements including those presented hereinpresented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) or the method of administration described in Schedule 1 are applied subject to the decision of the doctor overseeing the therapy.

Method Use of the Senolytic Peptide(s) for treatment of Neurological Diseases and Disorders

Chronic inflammation is a major contributor to a range of neurodegeneration the progressive dysfunction and loss of neurons in the central nervous system whereas neurodegeneration is the major cause of cognitive and motor dysfunction. While neuronal degeneration is well-known in Alzheimer's and Parkinson's diseases, it is also observed in neurotrophic infections, traumatic brain and spinal cord injury, stroke, neoplastic disorders, prion diseases, multiple sclerosis and amyotrophic lateral sclerosis. The adaptive immune response is implicated in neurodegenerative diseases contributing to tissue damage, but also plays important roles in resolving inflammation and mediating neuroprotection and repair. Therefore, the Senolytic Peptide(s) is suitable to slow down the progression of neurodegeneration by reducing the chronic inflammation in the body substantially by stopping SASP.

Senescence-associated diseases or disorders treatable by administering the Senolytic Peptide described herein include neurological diseases or disorders. Such senescence-associated diseases and disorders include Parkinson's disease, Alzheimer's disease (and other dementias), motor neuron dysfunction (MND), mild cognitive impairment (MCI), Huntington's disease, and diseases and disorders of the eyes, such as age-related macular degeneration.

The effectiveness of one or more Senolytic Peptides described herein and monitoring of a subject who receives one or more Senolytic Peptides can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of administering one or more Senolytic Peptides can be analyzed using techniques known in the art, such as comparing symptoms of subjects suffering from or at risk of Alzheimer's disease that have received the treatment with those of subjects without such a treatment or with placebo treatment.

In some embodiments, the Senolytic Peptide(s) is effective in the treatment of Alzheimer's disease. During the senolytic therapy the alzheimer's disease parameters which are measured include, inter alia, changes in ability to carry out daily activities, and changes in behavior and personality, tests of memory, problem solving, attention, counting, and language, blood and urine tests, brain scans, such as computed tomography (CT), magnetic resonance imaging (MRI), or positron emission tomography (PET) and/or biomarker analysis.

In some embodiments, the Senolytic Peptide(s) is effective in the treatment of Parkinson's disease. During the senolytic therapy the parkinson's disease parameters which are measured include, inter alia, analysis for tremors, limb or neck stiffness, general fitness and balance and/or locomotor function.

In some embodiments, the Senolytic Peptide(s) is effective in the treatment of depression. depression parameters that are to be measured are, inter alia, physical examination, sadness or depressed mood most of the day, major changes in weight, insomnia or excessive sleep, fatigue or loss of energy most of the day, feelings of hopelessness or worthlessness or excessive guilt, problems with concentration or decision making, recurring thoughts of death or suicide.

In some embodiments involving treatment of neurological diseases and Disorders Gentle regimes Regime is most suitable (FIG. 14C). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Impuse Regime or Sustained Regimes (FIG. 14A-B) or the method of administration described in Schedule 1 are applied subject to the decision of the doctor overseeing the therapy.

Method Use of the Senolytic Peptide(s) for Treatment of Ophthalmic Diseases and Disorders

Senescence-associated diseases or disorders treatable by administering the Senolytic Peptide described herein include ophthalmic diseases or disorders. Such ophthalmic diseases and disorders include but not limited to age-related macular degeneration, cataracts, glaucoma, vision loss, presbyopia. In some embodiments, Ophthalmic Diseases or Disorders involve age related macular degeneration (AMD) resulting in irreversible blindness which is associated with the degradation of retinal pigment epithelium (RPE) cells, photoreceptors, and choriocapillaris. Oxidative stress, inflammation (IL-17 involvement) and some genetic factors are known to be involved in AMD pathogenesis. Oxidative stress can induce DNA damage response (DDR), autophagy, and cell senescence.

Therefore, in some embodiments, the Senolytic Peptide(s) is suitable for treatment of premature senescence involved Ophthalmic Diseases and Disorders by removing the senescent cells in controlled and safe manner

In some embodiments, methods are provided herein for treating or preventing (i.e., reducing the likelihood of occurrence of; delaying the onset or development of, or inhibiting, retarding, slowing, or impeding progression or severity of) an ophthalmic disease, disorder, or condition (e.g., presbyopia, cataracts, macular degeneration); for selectively killing senescent cells in an eye of a subject, and/or inducing collagen production in the eye of a subject in need thereof by administering at least one Senolytic Peptide which may be combined with at least one therapeutically acceptable excipient to form a composition comprising the Senolytic Peptide(s)) directly to an eye.

In some embodiments involving treatment of Ophthalmic Diseases and Disorders Impulse Regime is most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) or the method of administration described in Schedule 1 are applied subject to the decision of the doctor overseeing the therapy.

Method Use of the Senolytic Peptide(s) for Treatment of Metabolic Diseases or Disorders

Senescence-associated diseases or disorders treatable by administering the Senolytic Peptide include metabolic diseases or disorders. Such senescence-associated diseases and disorders include diabetes, metabolic syndrome, diabetic ulcers, and obesity.

Over the past couple of decades, research has suggested that a chronic type of inflammation that affects the whole body is linked to diseases like type 2 diabetes. Diabetes is thought to be a consequence of chronic, low-grade inflammation, which appears to change the way glucose is absorbed by cells. Anakinra, a biologic anti-inflammatory drug, has been found to improve some diabetes symptoms by blocking the cytokine protein IL-1, as stated above the key instigator of the immune and inflammatory response. Therefore, the Senolytic Peptide(s) is suitable to slow down the progression of the diabetes by reducing the chronic inflammation in the body substantially by stopping SASP.

Subjects suffering from type 2 diabetes can be identified using standard diagnostic methods known in the art for type 2 diabetes. Generally, diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or physical examination of a subject. Subjects at risk of developing type 2 diabetes include those who have a family history of type 2 diabetes and those who have other risk factors such as excess weight, fat distribution, inactivity, race, age, prediabetes, and/or gestational diabetes.

The effectiveness of the Senolytic Peptide can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. A subject who is receiving one or more of the Senolytic Peptides described herein for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic (3-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass Index (BMI) is a measurement tool used to determine excess body weight, and is calculated from the height and weight of a subject. A human is considered overweight when the person has a BMI of 25-29; a person is considered obese when the person has a BMI of 30-39, and a person is considered severely obese when the person has a BMI of 40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorptiometry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly, on the lower extremities. Patients with diabetic venous ulcer exhibit elevated presence of cellular senescence at sites of chronic wounds. Chronic inflammation is also observed at sites of chronic wounds, such as diabetic ulcers suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more of cardiovascular disease, liver steatosis, hyperlipidemia, diabetes, and insulin resistance. A subject with metabolic syndrome may present with a cluster of metabolic disorders or abnormalities which may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

In some embodiments, the Senolytic Peptide(s) is effective in the treatment of diabetes type II. During the senolytic therapy, the diabetes type II disease parameters which are measured include, inter alia, basal blood glucose levels, average blood glucose levels over a period of time (2-3 months; A1C test), fasting plasma glucose, oral glucose tolerance test, plasma glucose test N.B.

In some embodiments, the Senolytic Peptide(s) is effective in the treatment of obesity. During the senolytic therapy the obesity parameters which are measured include alia body weight, Body-Mass-Index (BMI), waist circumference, waist-to-hip ratio, skinfold thicknesses, and bioelectrical impedance, magnetic resonance imaging anr/or dual energy X-ray absorptiometry.

In some embodiments, the Senolytic Peptide(s) is effective in the treatment of metabolic syndrome. During the senolytic therapy the metabolic syndrome disease parameters which are measured include, inter alia, measurements for obesity (see above, e.g. waist circumference), blood levels of triglicerides, HDL cholesterol, blood pressure, fasting glucose.

In some embodiments, the Senolytic Peptide(s) is effective in the treatment of hepatic insufficiency. During the senolytic therapy, the hepatic insufficiency disease parameters which are measured include, inter alia, blood AST and ALT values.

In some embodiments, the Senolytic Peptide(s) is effective in the treatment of cirrhosis. During the senolytic therapy, the cirrhosis disease parameters which are measured include, inter alia, measurements of blood-clotting factors and international normalized ratio for blood clotting, liver stiffness by magnetic resonance elastography, lver imaging by CT and/or MRI, physical examination, blood testing for bilirubin and creatinine, and/or liver biopsy analysis for liver damage.

In some embodiments involving treatment of metabolic diseases disorders Impulse Regime is most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) or the method of administration described in Schedule 1 are applied subject to the decision of the doctor overseeing the therapy.

Method Use of the Senolytic Peptide(s) for treatment of Renal Dysfunction:

Nephrological pathologies, such as glomerular disease, arise in the elderly. Glomerulonephritis is characterized by inflammation of the kidney and by the expression of two proteins, IL1α and IL1β. IL1α and IL1β are considered master regulators of SASP. Glomerular disease is associated with elevated presence of senescent cells, especially in fibrotic kidneys. Therefore, the Senolytic Peptide(s) is suitable to renal dysfunction by substantially stopping SASP by killing the senescent cells.

In some embodiments, the Senolytic Peptide(s) is effective in the treatment of renal insufficiency. During the senolytic therapy, the renal insufficiency disease parameters which are measured include, inter alia, blood pressure, heart/lung sound analysis, nervous system exam, urinalysis for protein content, analysis for creatinine clearance and level of Blood Urea Nitrogen, CT, MRI and/or ultrasound of abdomen and kidneys, kidney biopsy for damage analysis.

Glomerulosclerosis is another pathology associated with renal aging supported by the accumulation of senescent cells as indicated by an increase in the levels of senescence markers such as p16 and SA-β-Gal by aging.

In some embodiments, renal dysfunction is as a result of nephrological pathologies, such as glomerulosclerosis. Senolytic Peptide(s) is effective in the treatment of glomerulosclerosis. During the senolytic therapy, the glomerulosclerosis disease parameters which are measured include, inter alia, swellings in limbs, weight gains, changes in urine due to proteinuria, distortion or compression of the small capillaries in the glomerulus that filter blood in a biopsy and plasma Urea or protein concentration, blood pressure, glomerular filtration rate, and/or kidney ultrasound.

In some embodiments involving treatment of renal dysfunction and disorders Impulse Regime is most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) or the method of administration described in Schedule 1 are applied subject to the decision of the doctor overseeing the therapy.

Method Use of the Senolytic Peptide(s) as an Adjuvant Agent in a Cancer Therapy and for Preventing Metastasis

Stress-induced premature senescence (SIPS) occurs rapidly in response to various stresses such as chemotherapeutic drugs and ionizing radiation. Both stresses cause substantial collateral macromolecular damage to non-neoplastic cells and responsible for the early aging phenotypes frequently observed in cancer survivors. Contrary to chronic senescence resulting from normal aging mechanisms and declining macromolecular repair mechanisms, therapy-induced senescence results from abrupt exogenous stresses placed on tissues during cancer therapy. The Senolytic Peptide(s) may be administered to the subjects who may also have cancer, not as a primary cure but as an adjuvant to prevent metastasis using the methods as described herein. Metastasis of a cancer occurs when the cancer cells (e.g., tumor cells) spread beyond the anatomical site of origin and initial colonization to other areas throughout the body of the subject.

In some embodiments, the subject may be considered in partial or complete cancer remission wherein methods for described herein for killing senescent cells are not intended as a primary treatment for cancer. In some embodiments, said subjects exhibit certain levels of cell degeneration that may eventually lead to cancer.

In one embodiment, methods are provided for preventing (i.e., reducing the likelihood of occurrence of), inhibiting, or retarding metastasis in a subject who has a cancer by administering the Senolytic Peptide as described herein. In some embodiments, the Senolytic Peptide is administered as described in the Gentle regimes Regime within a treatment window (i.e., treatment course).

Such the Senolytic Peptide when administered in a Therapeutically Effective Dose to a subject who has a cancer according to the methods described herein may inhibit tumor proliferation.

The methods described herein are also useful for inhibiting, retarding or slowing progression of metastatic cancer of any one of the types of tumors described in the medical art.

The methods described herein are also useful for inhibiting, retarding or slowing progression of metastatic cancer of any one of the types of tumors described in the medical art.

In some embodiments, the cancer type is but not limited to metastatic melanoma, resistant breast cancer or radiotherapy resistant glioblastoma.

Senolytic Peptide(s) is effective in the treatment of metastatic melanoma or as an adjuvant drug. During the senolytic therapy, the metastatic melanoma disease parameters which are measured include, inter alia, a reduction in tumor size and/or metastazation.

In some embodiments, the Senolytic Peptide(s) is effective in the treatment of resistant breast cancer or as an adjuvant drug. During the senolytic therapy, the resistant breast cancer disease parameters which are measured include a reduction in tumor size and/or metastazation.

In some embodiments, the Senolytic Peptide(s) is effective in the treatment of a resistant glioblastoma or as an advuvant drug. During the senolytic therapy, the resistant glioblastoma disease parameters which are measured include, inter alia, a reduction in tumor size and/or metastazation.

In some embodiments wherein the treatment with the Senolytic Peptide(s) aims the inhibition of metastasis in a subject who has a cancer the Sustained Regime (FIG. 14B) is most suitable. In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Impulse Regime or Gentle Regimes (FIG. 14A,C) or the method of administration described in Schedule 1 are applied subject to the decision of the doctor overseeing the therapy.

Method of Use of the Senolytic Peptide(s) for treatment of Progeroid Syndromes

Progeroid syndromes (PSs) are a group of fatal, severe and rare genetic disorders which mimic premature aging while exhibiting various clinical features and phenotypes. PSs mimic many of the characteristics of human ageing such as hair loss, short stature, skin tightness, cardiovascular diseases and osteoporosis. Therefore, the Senolytic Peptides may also be useful for treating or alleviation of the effects of Progeroid Syndromes that occur as a result of premature aging process induced by congenital genetic mutations in individuals. Although all progeroid syndromes are characterized by similar clinical features, their underlying mechanisms can vary depending on the mutated gene and the pathway that is consequently altered. As a result of genomic instabilities due to the mutated genes, premature senescence emerges as a key factor underlying these conditions.

In some embodiments, these syndromes include clinically and genetically heterogeneous diseases such as ataxia-telangiectasia, Bloom syndrome, Cockayne syndrome, Fanconi anaemia, Hutchinson-Gilford Progeria syndrome, Rothmund-Thomson syndrome, trichothiodystrophy, xeroderma pigmentosum, and Werner syndrome (aka adult progeria).

In some embodiments, Progeroid Syndromes include Hutchinson-Gilford Progeria Syndrome (HGPS) which is a rare , fatal and genetic condition of childhood, characterized by growth reduction, failure to thrive, a typical facial appearance (prominent forehead, protuberant eyes, thin nose with a beaked tip, thin lips, micrognathia and protruding ears) and distinct dermatologic features (generalized alopecia, aged-looking skin, sclerotic and dimpled skin over the abdomen and extremities, prominent cutaneous vasculature, dyspigmentation, nail hypoplasia and loss of subcutaneous fat). Individuals with HGPS exhibit atherosclerosis, lipodystrophy, heart infarction and death during puberty.

During the senolytic treatment HGPS disease parameters are measured by, inter alia, features of accelerated aging, hair loss (alopecia), aged-looking skin, joint abnormalities, and a loss of fat under the skin.

In some embodiments, Progeroid Syndromes include Trichothiodystrophy characterized by brittle hair causing hair loss, neurological defects, bone abnormalities and fitness decline.

In other embodiments, PSs include Werner Syndrome characterized by the dramatic, rapid appearance of features associated with normal aging in affected individuals. Affected individuals usually develop accopanying disorders of aging early in life, such as cataracts, skin ulcers, type 2 diabetes, diminished fertility, atherosclerosis, osteoporosis, and some types of cancer.

In some embodiments, premature aging-associated decline and symptoms may be treated or prevented (i.e., the likelihood of occurrence of is reduced) by administering the Senolytic Peptide.

In some embodiments involving treatment or alleviation of Progeroid Syndromes the Impulse Regime is most suitable (FIG. 14A). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regime (FIG. 14B-C) or the method of administration described in Schedule 1 are applied subject to the decision of the doctor overseeing the therapy.

The effectiveness of a method of treatment described herein may be manifested by reducing the number of symptoms of a premature aging disease or progeroid trait associated with a senescence-inducing stimulus, decreasing the severity of one or more symptoms, or delaying the progression of a premature aging disease or progeroid trait associated with a senescence-inducing stimulus. In some embodiments, preventing a premature aging disease or progeroid trait associated with a senescence-inducing stimulus refers to preventing (i.e., reducing the likelihood of occurrence) or delaying onset of a premature aging disease or progeroid trait associated with a senescence-inducing stimulus, or reoccurrence of one or more premature aging disease or progeroid trait associated with a senescence-inducing stimulus.

Method of Use of the Senolytic Peptide(s) to Reduce the Side Effects of Chemotherapy and Radiotherapy

Tumor proliferation may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells. It has been demonstrated that a senolytic agent lowers the threshold for senescent cells to enter apoptosis after DNA damage. In some embodiments the Senolytic Peptide(s) is used as a senolytic agent against chemotoxicity or radiation damage by removing the senescent cells in controlled and safe manner

Because cells may be induced to senesce by cancer therapies, such as radiation and certain chemotherapy drugs (e.g., doxorubicin; paclitaxel; gemcitabine; pomalidomide; lenalidomide), the Senolytic Peptide(s) described herein may be administered after the chemotherapy or radiotherapy to kill (or facilitate killing) of these senescent cells.

As discussed herein and understood in the art, establishment of senescence, such as shown by the presence of a senescence-associated secretory phenotype (SASP), occurs over several days; therefore, administering the Senolytic Peptide to kill senescent cells, and thereby reduce the likelihood of occurrence or reduce the extent of metastasis, is initiated when senescence has been established. As discussed herein, the following treatment courses for administration of the Senolytic Peptide may be used in methods described herein for treating or preventing (i.e., reducing the likelihood of occurrence, or reducing the severity) a chemotherapy or radiotherapy side effect. Removal or destruction of senescent cells may ameliorate acute toxicity, including acute toxicity comprising energy imbalance, of a chemotherapy or radiotherapy. Acute toxic side effects include but are not limited to gastrointestinal toxicity (e.g., nausea, vomiting, constipation, anorexia, diarrhea), peripheral neuropathy, fatigue, malaise, low physical activity, hematological toxicity (e.g., anemia), hepatotoxicity, alopecia (hair loss), pain, infection, mucositis, fluid retention, dermatological toxicity (e.g., rashes, dermatitis, hyperpigmentation, urticaria, photosensitivity, nail changes), mouth (e.g., oral mucositis), gum or throat problems, or any toxic side effect caused by a chemotherapy or radiotherapy.

Accordingly, in some embodiments, methods are provided herein for ameliorating (reducing, inhibiting, or preventing occurrence (i.e., reducing the likelihood of occurrence)) acute toxicity or reducing severity of a toxic side effect (i.e., deleterious side effect) of a chemotherapy or radiotherapy or both in a subject who receives the therapy, wherein the method comprises administering to the subject an agent that selectively kills, removes, or destroys or facilitates selective destruction of senescent cells. Administration of the Senolytic Peptide for treating or reducing the likelihood of occurrence, or reducing the severity of a chemotherapy or radiotherapy side effect may be accomplished by the same treatment courses described above for treatment/prevention of metastasis. As described for treating or preventing (i.e., reducing the likelihood of occurrence of) metastasis, the Senolytic Peptide is administered in a Therapeutically Effective Dose during the off-chemotherapy or off-radiotherapy time interval or after the chemotherapy or radiotherapy treatment regimen has been completed.

In some embodiments wherein the treatment aims the reduction of the side effects of chemotherapy and radiotherapy the Sustained Regime is most suitable (FIG. 14B). In various embodiments the Therapeutically Effective Dose and/or the duration of treatment of above stated regimes could be altered depending on the subject's state of health and/or the subject's response to the treatment. During the therapy, a subject's progress can be monitored by appropriate measurements including those presented herein or by detecting the senescent cell population on a biopsy sample taken from the subject at the beginning and throughout the period of the therapy and thus monitoring the senescent cell population decline. In various embodiments when the subject has other diseases or disorders, an individualized treatment course of the Sustained Regime or Gentle Regimes (FIG. 14B-C) are applied subject to the decision of the doctor overseeing the therapy.

The number of cycles of a chemotherapy or radiotherapy or the total length of time of a chemotherapy or radiotherapy dose regime can vary depending on the subject's response to the cancer therapy. In some embodiments the senolytic therapy plan timeframe will be adjusted and aligned with said chemotherapy or radiotherapy treatment by a person skilled in the oncology art.

Compositions and Methods of Delivery for the Senolytic Peptide

In some embodiments, compositions comprising a Senolytic Peptide can be formulated in a manner appropriate for the delivery method by using techniques routinely practiced in the art. The composition may be in the form of a solid (e.g., tablet, capsule), semi-solid (e.g., gel), liquid, or gas (aerosol). In some embodiments, the Senolytic Peptide (or a composition comprising same) is administered in a Therapeutically Effective Dose as a bolus infusion. In some embodiments when the Senolytic Peptide is delivered in a Therapeutically Effective Dose by infusion wherein the Senolytic Peptide is delivered to an organ or tissue comprising senescent cells to be killed via a blood vessel in accordance with techniques routinely performed by a person skilled in the medical art.

Pharmaceutically-acceptable excipients are well-known in the pharmaceutical arts. Examples of pharmaceutically-acceptable excipients include sterile saline and phosphate buffered saline at physiological pH. Preservatives, stabilizers, dyes, buffers, and the like may be provided in the composition. In general, the type of excipient is selected based on the mode of administration, as well as the chemical composition of the active ingredient(s). Alternatively, the Senolytic Peptide may be formulated as a lyophilizate. A composition described herein may be lyophilized or otherwise formulated as a lyophilized product using one or more appropriate excipient solutions for solubilizing and/or diluting the agent(s) of the composition upon administration. In some embodiments, the Senolytic Peptide may be encapsulated within liposomes using technology known and practiced in the art. Compositions comprising the Senolytic Peptide may be formulated for any appropriate manner of administration described herein and in the art.

A composition comprising the Senolytic Peptide(s) may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocularly, vaginally, rectally, or by intracranial injection, or by some combination thereof. In some embodiments, administration of a dose, as described above, is via intravenous, intraperitoneal, directly into the target tissue or organ, or subcutaneous route. In some embodiments, a delivery method includes drug-coated or permeated stents for which the drug is the Senolytic Peptide. Formulations suitable for such delivery methods are described in greater detail herein.

In some embodiments, the Senolytic Peptide (which may be combined with at least one therapeutically-acceptable excipient to form a composition comprising the Senolytic Peptide(s)) is administered in a Therapeutically Effective Dose directly to the target tissue or organ comprising senescent cells that contribute to manifestation of the disease or disorder. In some embodiments when treating osteoarthritis, at least one Senolytic Peptide is administered in a Therapeutically Effective Dose directly to an osteoarthritic joint (i.e., intra-articularly) of a subject in need thereof. In some embodiments, the Senolytic Peptide(s) may be administered in a Therapeutically Effective Dose to the joint via topical, transdermal, intradermal, or subcutaneous route. In some embodiments, methods are provided herein for treating a cardiovascular disease or disorder associated with arteriosclerosis, such as atherosclerosis by administering directly into an artery. In some embodiments, the Senolytic Peptide (which may be combined with at least one pharmaceutically-acceptable excipient to form a composition comprising the Senolytic Peptide(s)) for treating a senescence-associated pulmonary disease or disorder may be administered in a Therapeutically Effective Dose by inhalation, intranasally, by intubation, or intracheally, for example, to provide the Senolytic Peptide more directly to the affected pulmonary tissue. By way of another non-limiting example, the Senolytic Peptide (or composition comprising the Senolytic Peptide) may be delivered directly to the eye either by injection (e.g., intraocular or intravitreal) or by conjunctival application underneath an eyelid of a cream, ointment, gel, or eye drops. In some embodiments, the Senolytic Peptide or a composition comprising the Senolytic Peptide may be formulated as a timed release (also called sustained release, controlled release) composition or may be administered in a Therapeutically Effective Dose as a bolus infusion.

A composition comprising the Senolytic Peptide(s) (e.g., for oral administration or for injection, infusion, subcutaneous delivery, intramuscular delivery, intraperitoneal delivery or other method) may be in the form of a liquid. A liquid composition comprising the Senolytic Peptide(s) may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable composition comprising the Senolytic Peptide(s) is preferably sterile. In another embodiment, for treatment of an ophthalmological condition or disease, a liquid composition comprising the Senolytic Peptide(s) may be applied to the eye in the form of eye drops. A liquid composition comprising the Senolytic Peptide(s) may be delivered orally.

For oral formulations, at least one of the Senolytic Peptides described herein can be used alone or in combination with appropriate additives to make tablets, powders, granules or capsules, and if desired, with diluents, buffering agents, moistening agents, preservatives, coloring agents, and flavoring agents. The compounds may be formulated with a buffering agent to provide for protection of the compound from low pH of the gastric environment and/or an enteric coating. The Senolytic Peptide included in a composition comprising the Senolytic Peptide(s) may be formulated for oral delivery with a flavoring agent, e.g., in a liquid, solid or semi-solid formulation and/or with an enteric coating.

A composition comprising any one of the Senolytic Peptides described herein may be formulated for sustained or slow release (also called timed release or controlled release). Such compositions comprising the Senolytic Peptide may generally be prepared using well known technology and administered in a Therapeutically Effective Dose by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active agent contained within a sustained release formulation depends upon the site of implantation, the rate and expected duration of release, and the nature of the condition, disease or disorder to be treated or prevented.

In some embodiments, the compositions comprising the Senolytic Peptide are formulated for transdermal, intradermal, or topical administration. Said compositions can be administered in a Therapeutically Effective Dose using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered in a Therapeutically Effective Dose topically or injected directly into the skin adjacent to or within the area to be treated (intradermally or subcutaneously). The active compositions comprising the Senolytic Peptide can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Compositions comprising the Senolytic Peptide can be formulated as emulsions for topical application. An emulsion contains one liquid distributed within the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically-approved excipients. Suitable surfactants include, but are not limited to, anionic surfactants, non-ionic surfactants, cationic surfactants, and amphoteric surfactants. Compositions comprising the Senolytic Peptide for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions comprising the Senolytic Peptide, patches, bandages and articles. These systems are semisolid emulsions, micro-emulsions, or foam emulsion systems.

In some embodiments, the Senolytic Peptide(s) can be formulated with oleaginous bases or ointments to form a semisolid composition with a desired shape. In addition to the Senolytic Peptide, these semisolid compositions comprising the Senolytic Peptide can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

In some embodiments, a composition comprising any one of the Senolytic Peptides described herein may be formulated for sustained or slow release (which may also be called timed release or controlled release). Controlled or sustained release transdermal or topical formulations can be achieved by the addition of time-release additives, such as polymeric structures, matrices, that are available in the art. For example, the compositions comprising the Senolytic Peptide may be administered in a Therapeutically Effective Dose through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the active agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. Rate controlling polymers may be useful for administration to sites where pH change can be used to effect release. These rate controlling polymers can be applied using a continuous coating film during the process of spraying and drying with the active compound. In one embodiment, the coating formulation is used to coat pellets comprising the active ingredients that are compressed to form a solid, biodegradable insert.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the Senolytic Peptide. The matrices can be formed by solvent evaporation, spray drying, solvent extraction and other methods known to those skilled in the art.

In some embodiments of a method described herein for treating a cardiovascular disease associated with or caused by arteriosclerosis, one or more Senolytic Peptide(s) may be delivered directly into a blood vessel (e.g., an artery) via a stent. In some embodiments, a stent is used for delivering the Senolytic Peptide to an atherosclerotic blood vessel (an artery). Several methods are described in the art for preparing drug-coated and drug-embedded stents. In some embodiments the Senolytic Peptide may also be incorporated into the stent (for example as a coating or pores in the metal stent itself). In some embodiments, the Senolytic Peptide may be formulated within liposomes and applied to a stent. Placement of stents in an atherosclerotic artery is performed by a person skilled in the medical art.

In some embodiments, the Senolytic Peptide is administered in a Therapeutically Effective Dose to a subject who has an ophthalmic senescence-associated or disease or disorder may be delivered intraocularly or intravitreally. In other some embodiments, the Senolytic Peptide(s) may be administered in a Therapeutically Effective Dose to the eye by a conjunctival route, applying the Senolytic Peptide to the mucous membrane and tissues of the eye lid, either upper, lower, or both. Any of these administrations may be bolus infusions.

In some embodiments, the Senolytic Peptide(s) is administered in a Therapeutically Effective Dose in the form of PEGylated peptide. PEGylation is an alternative route for some peptides which could not be cyclic Amphiphilicity of PEG increases the solubility of the said peptides in both organic solvents and water. Direct PEGylation of peptides increase absorption and systemic stability of said peptides. Additionally, PEG and its metabolites are non-toxic at the concentrations that are used for peptide delivery. As will be recognized by those skilled in the art the Senolytic Peptide(s) could be PEGylated. In some embodiments of the Senolytic Peptide(s) is PEGylated.

In some embodiments, the fatty acid conjugated Senolytic Peptide(s) is administered in a Therapeutically Effective Dose. Lipidization has been used to increase protein bioavailability during oral administration. Conjugation of the polypeptides with fatty acids improves the transport across membranes and confers the peptide with higher stability and half-life. As will be recognized by those skilled in the art the Senolytic Peptide(s) could be conjugated with fatty acids by lipidization. In some embodiments of the Senolytic Peptide(s) is conjugated with fatty acids by lipidization.

In some embodiments, vitamin B12 conjugated Senolytic Peptide(s) is administered in a Therapeutically Effective Dose. Conjugation of peptides to vitamin B12 and its derivatives is a method to increase oral absorption said peptides by exploiting the receptor mediated absorption of vitamin B12 bound to intrinsic factor. As will be recognized by those skilled in the art the Senolytic Peptide(s) could be conjugated with vitamin B12 or its derivatives. In some embodiments of the Senolytic Peptide(s) is conjugated with vitamin B12 or its derivatives.

In some embodiments, stapled Synthetic Peptide(s) is administered in a Therapeutically Effective Dose. Stapled peptides have an alpha helical structure wherein various residues are linked by a synthetic hydrocarbon backbone. They have been used in the drug delivery to improve the biochemical properties of the delivered peptides by locking the peptide conformation, increasing the helicity and solution stability of the said peptides. As will be recognized by those skilled in the art the Senolytic Peptide(s) could be synthesized as stapled peptides. In some embodiments of the Senolytic Peptide(s) is synthesized as stapled peptides.

In some embodiments, N or C-terminally modified Synthetic Peptide(s) is administered in a Therapeutically Effective Dose. N and/or C terminal modification of peptides can confer stability by increasing resistance to proteolysis. N-acetylation and C-amidation have been exhibited to improve resistance to proteolytic degradation. As will be recognized by those skilled in the art the Senolytic Peptide(s) could be N-acetylated or C-amidated. In some embodiments of the Senolytic Peptide(s) is N-acetylated or C-amidated.

In some embodiments, the Senolytic Peptide(s) is administered in a Therapeutically Effective Dose as Prodrugs. Prodrugs are derived from drug molecules. They are bioreversible compounds which can release the active parent drug in vivo through enzymatic or chemical transformation. As will be recognized by those skilled in the art the Senolytic Peptide(s) could be synthesized as Prodrug peptides. In some embodiments of the Senolytic Peptide(s) is synthesized as Prodrug peptides.

As will be recognized by those skilled in the art in some embodiments the Senolytic Peptide(s) could be co-administered with enzyme inhibitors to alleviate proteolytic degradation thereby increasing bioavailability. In some embodiments of the Senolytic Peptide(s) is co-administered with enzyme inhibitor.

As will be recognized by those skilled in the art in some embodiments, the Senolytic Peptide(s) could be co-administered with absorption enhancers such as chitin and its derivatives such as chitosan to enhance absorption of hydrophilic drug molecules. In some embodiments of the Senolytic Peptide(s) is co-administered with absorption enhancers such as chitin and its derivatives (i.e. chitosan).

### EXAMPLES

### Example 1

Senolytic Peptide(s) Selectively Clears Senescent Cells

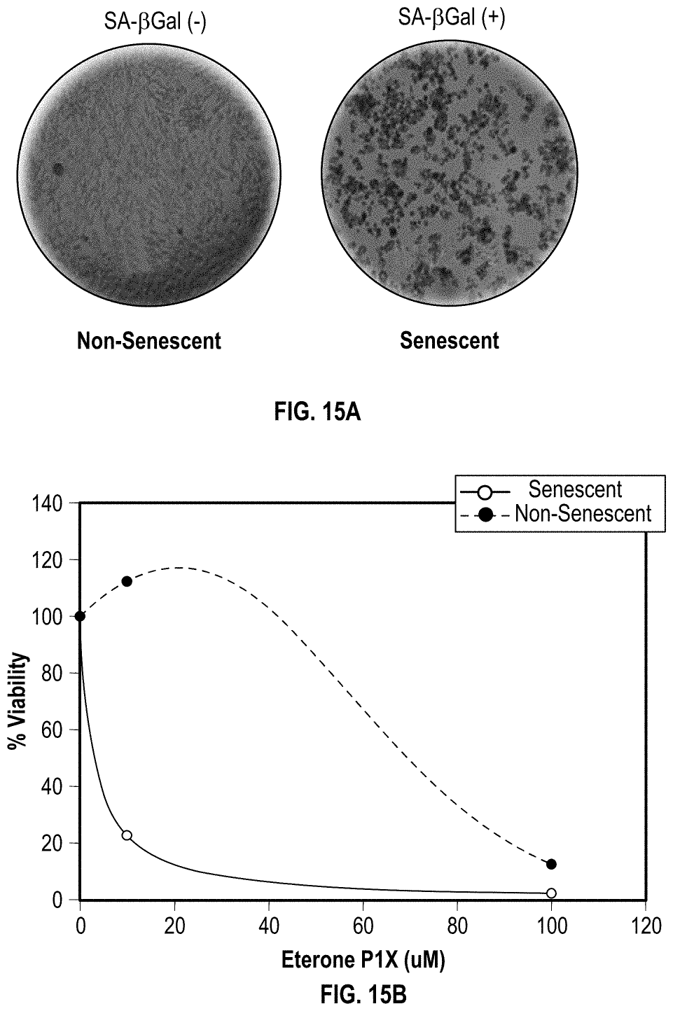

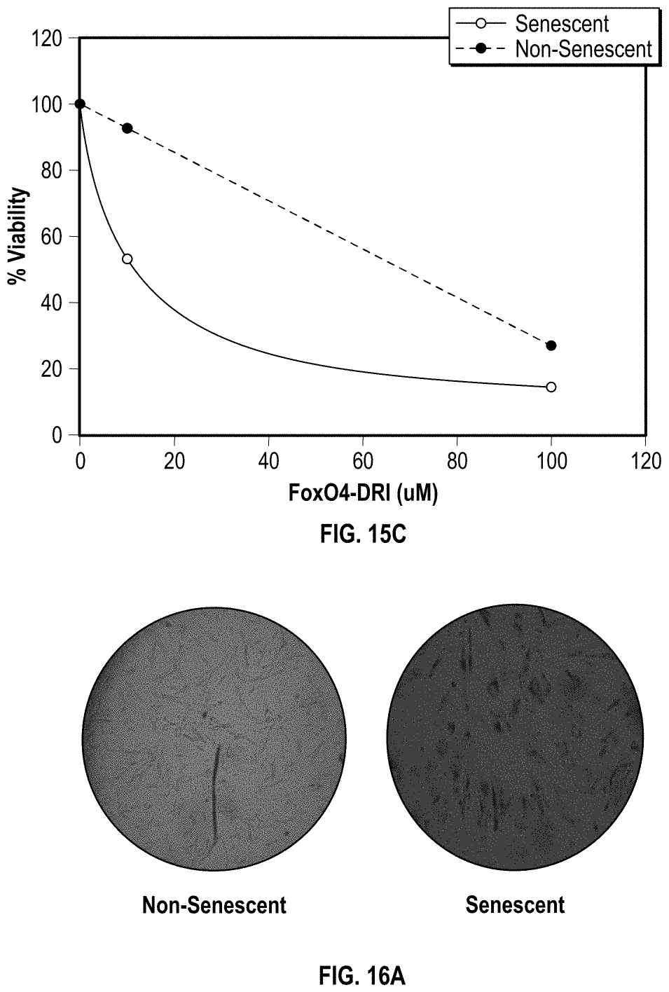

IMR-90 cells (ATCC #CCL-186, a diploid primary human fibroblast adherent cell line derived from fetal lung tissue) were treated with 100 NM Doxorubicin twice every other day for senescence induction. After 5 days, the cells were assayed for Senescece-Associated B-Galactosidase (SA-B-Gal) activity (Senescence Detection Kit, Abcam) and confirmed to possess SA-B-Gal activity. Later these cells were used for experiments comparing them to their non-Doxorubicin (non-senescent) treated counterparts. Senescent and non-senescent IMR90 fibroblasts were plated for XTT viability assays. 7000 senescent (obtained as described above) and 2000 non-senescent IMR90 fibroblasts were plated in 96-well plates incubated. The cells were treated with mock (PBS) or the Senolytic Peptide(s) at total doses ranging from 0 to 100 pM. The mock and peptide treatments were carried out for 3 consecutive days (24, 48 and 72 hours) after plating. After treatment period, the cells were incubated for 7 days until XTT-based viability was analyzed according to the manufacturer's instructions. The senescent cells showed much steeper decrease in viability compared with the non-senescent cells. The Senolytic Peptide(s) were ordered from Chinese Peptide Company with TFA removal. The Senolytic Peptide (SEQ ID NO: 11) is formed of L-amino acids, with a single D amino acid insertion to the N-terminus for enhancing bioavailibility. The Senolytic Peptide SEQ ID NO: 12 is formed of all L amino acids, with the addition of HIV-TAT sequence to the N-terminus for intracellular delivery. The Senolytic Peptide(s) were computationally designed to interfere with the FoxO4 CR3 domain to liberate p53 from the FoxO4-p53 complex. The Senolytic Peptide (SEQ ID NO: 11) selectively decreased the viability of senescent cells but not non-senescent cells (FIG. 15A). The senolytic effect of the Senolytic Peptide (SEQ ID NO: 11) was superior to FoxO4-DRI as shown by a sharper decrease in viability curves of the senescent IMR90 cells (FIG. 15B).

### Example 2

Chemotherapy (Doxorubicin) Induces Senescence In Vitro Which is Counteracted by the Senolytic Peptide(s)

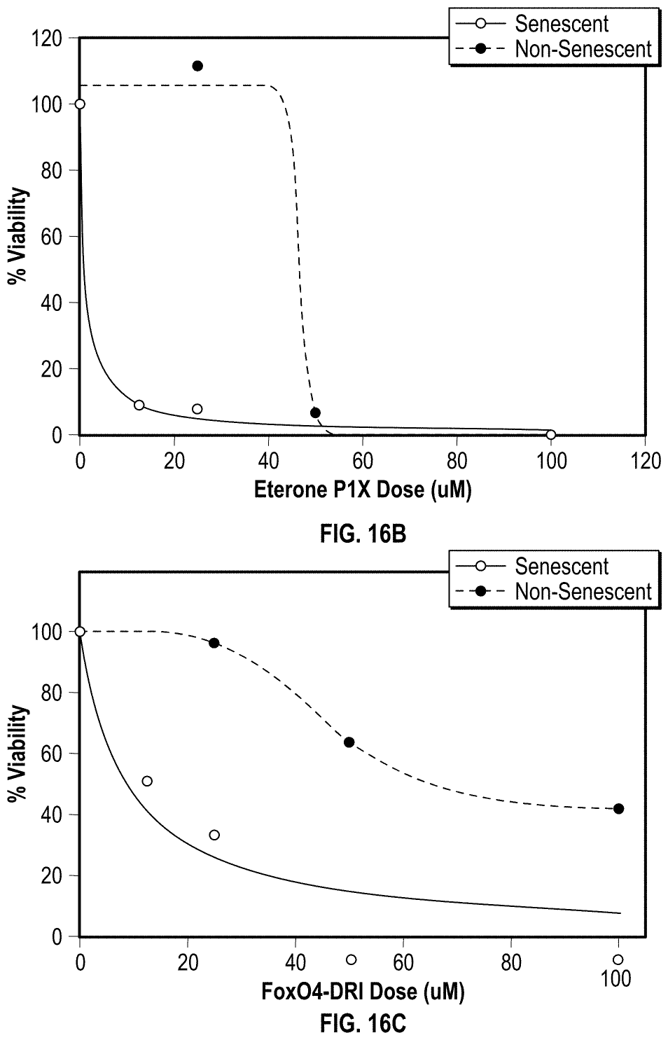

WI-38 cells (ATCC #CCL-75, a diploid primary human fibroblast adherent cell line derived from fetal lung tissue) were treated with a chemotherapy agent, 100 NM Doxorubicin twice every other day to induce senescence. After 5 days, the cells were assayed for Senescence-Associated B-Galactosidase (SA-B-Gal) activity (Senescence Detection Kit, Abcam) and confirmed to possess SA-B-Gal activity. Later these cells were used for experiments comparing them to their non-Doxorubicin (non-senescent) treated counterparts. Senescent and non-senescent WI-38 fibroblasts were plated for XTT viability assays. 7000 senescent (obtained as described above) and 2000 non-senescent WI-38 fibroblasts were plated in 96-well plates incubated. The cells were treated with mock (PBS) or the Senolytic Peptide (SEQ ID NO: 11) at total doses ranging from 0 to 100 pM. The mock and peptide treatments were carried out for 3 consecutive days (24, 48 and 72 hours) after plating. After treatment period, the cells were incubated for 7 days until XTT-based viability was analyzed according to the manufacturer's instructions. The senescent cells showed much steeper decrease in viability compared with the non-senescent cells. The Senolytic Peptide (SEQ ID NO: 11) was ordered from Chinese Peptide Company with TFA removal. Senolytic Peptide (SEQ ID NO: 11) selectively decreased the viability of senescent cells but not non-senescent cells (FIG. 16A). The senolytic effect of the Senolytic Peptide (SEQ ID NO: 11) was superior to FoxO4-DRI as shown by a sharper decrease in viability curves of the senescent WI-38 cells (FIGS. 16B-C) as reflected by SI50 values from these viability curves. SI50 value of the Senolytic Peptide (SEQ ID NO: 11) and FoxO4-DRI were found to be 4.9 and 29.5, respectively.

### Example 3

Treatment of Chemotherapy Induced Renal Senescence/Treatment of Senescence-Associated Renal Diseases

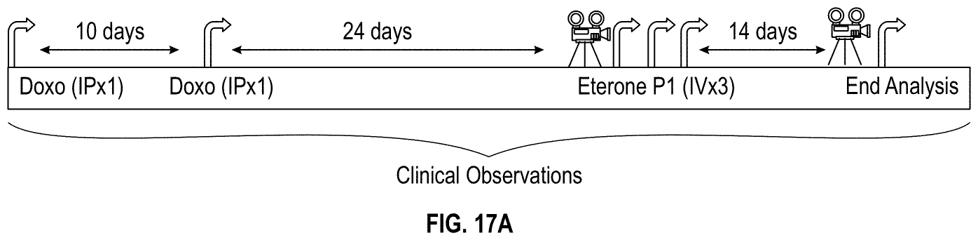

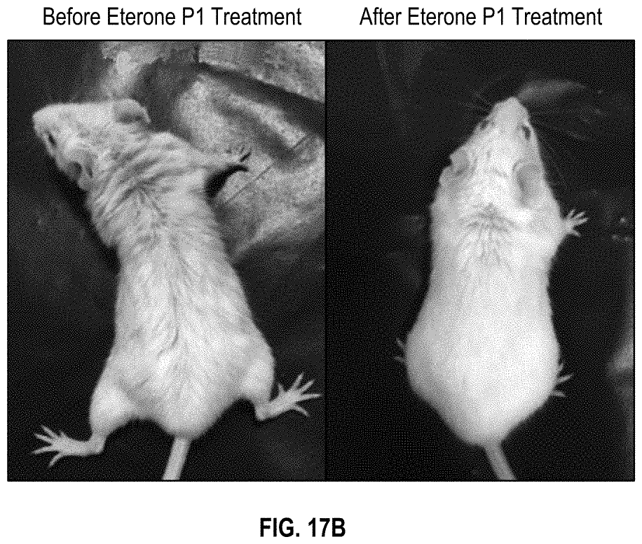

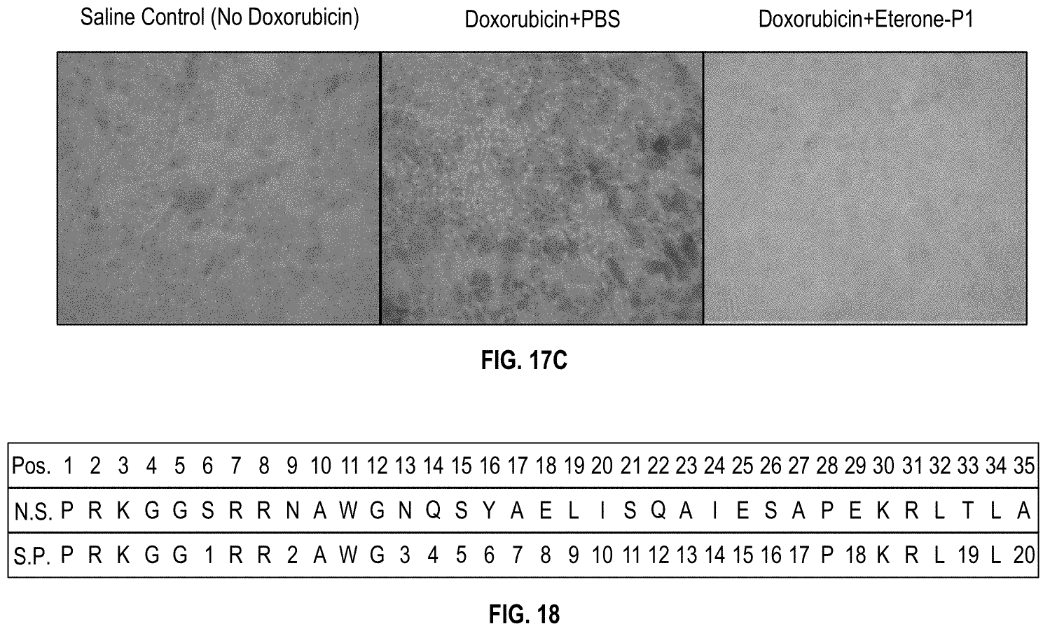

This study was performed in strict accordance with the protocol approved by the TUBITAK-MAM Animal Ethics Committee. All the mice used in this study were of a BALB/c background at 8-12 weeks of age. All mice were kept in group housing until the start of the experiment after which they were placed in separate cages (4 mice per cage). Only male mice were used throughout the study. Where feasible, littermates were used. All mice were randomly assigned to experimental group. Mice were fed ad libidum. Initial weights of the mice were recorded. Mice were divided into three groups (A-No Treatment, B-Doxorubicin Treatment+Mock Treatment, C-Doxorubicin Treatment+Senolytic Peptide Treatment). Doxorubicin induced chemotoxicity was used for the induction of renal senescence in mice. Mice in Group B and C were intraperitoneally administered with doxorubicin (8 mg/kg) twice (at 0 and 10th day). Weights of the mice were recorded three times a week. 24 days after 2^{nd} Doxorubicin treatment, PBS (mock Treatment) was administered intravenously to Group B and Senolytic Peptide (SEQ ID NO: 12) (3×10 mg/kg) was administered to Group C in three days (every other day) (FIG. 17A). The mice health, activity and appearance were monitored and they were sacrificed 14 days later. Gross pathology and pathology of the mice analysed. Mice organs and tissues are flash-frozen in liquid nitrogen and embedded in OCT and kept at −80 C. 10 micron thick mid-sagittal cryo-sections of the kidney were taken and SA-B-Gal activity was assayed as in Example 1-3. Doxorubicin induced alopecia and caused weight loss in mice and both conditions are reversed upon the Senolytic Peptide (SEQ ID NO: 12) Treatment (FIG. 17B). Doxorubicin induced renal senescence in BALB/c as corroborated by SA-B-Gal activity and treatment with the Senolytic Peptide cleared senescent cells in kidneys of doxorubicin treated mice (FIG. 17C).

### Example 4

Senolytic Peptide(s) is Non-Toxic at Therapeutically Effective Doses

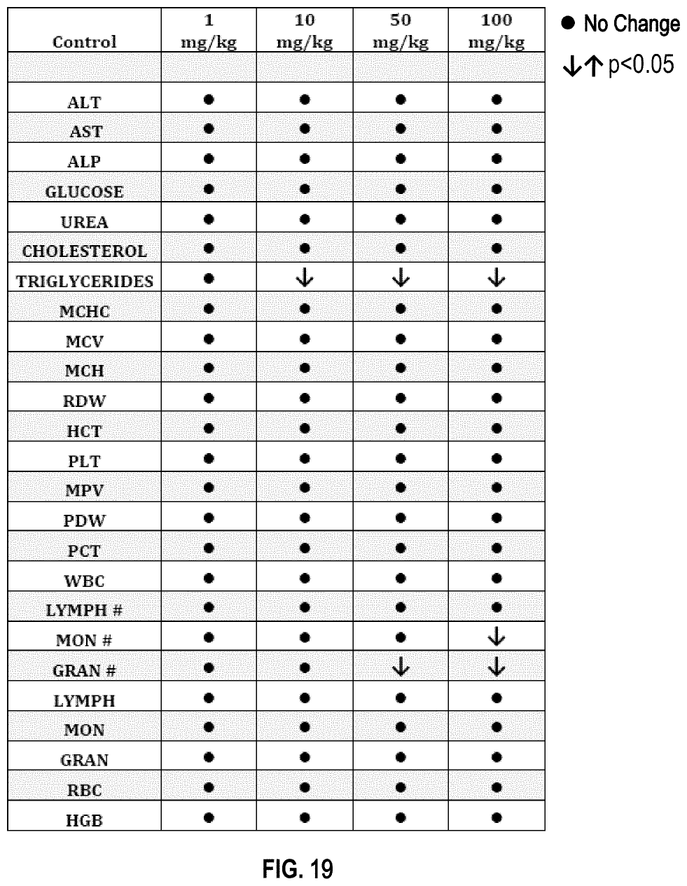

Senolytic Peptides were evaluated for toxicity according to Acute Sytemic Toxicity Test (ISO 10993-11). This study was performed in strict accordance with the protocol approved by the TUBITAK-MAM Animal Ethics Committee. All the mice used in this study were of a BALB/c background at 8-12 weeks of age. All mice were kept in group housing until the start of the experiment after which they were placed in separate cages (5 mice per cage). The Senolytic Peptide (SEQ ID NO: 11) is administered intravenously using a mice model (BALB/c) at doses of 1, 10, 50 and 100 mg/kg and vehicle alone is also administered using single injections (n=5, per group). Control and Senolytic Peptide (SEQ ID NO: 11) injected mice were sacrificed 24 hours later for pathological examination. No toxicity to the physical appearance of the mice was observed within 24 hours of injection. Blood samples were subsequently collected for determination and analysis of hematological and biochemical parameters. Plasma alanine aminotransferase (ALT), aspartate aminotransferase (AST), alkaline phosphatase (ALP) activities and blood urea, glucose, triglyceride levels were determined, inter alia, full hematological parameters. The results indicated that even at higher doses (100 mg/kg) than doses used in example 3, hematological parameters, AST, ALT, ALP levels and gross pathology results indicate no toxicity to the said mice model (FIG. 19).

### Example 5

Chemotherapy Induced Senescence In Vitro is Counteracted by the Screened Senolytic Peptide(s)

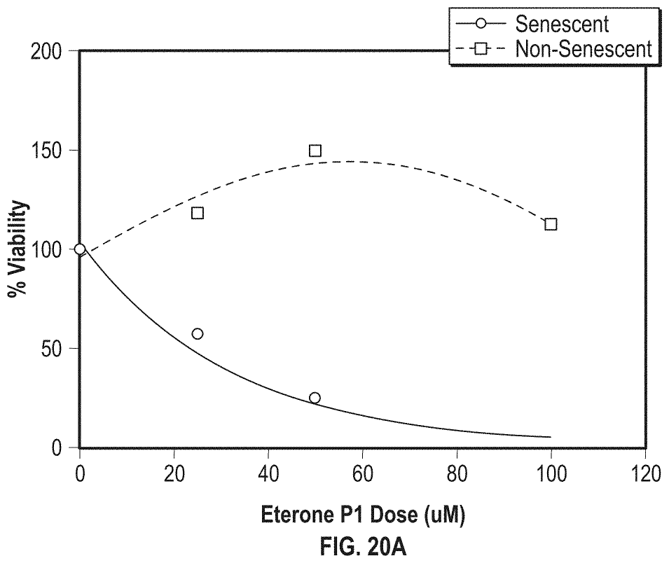

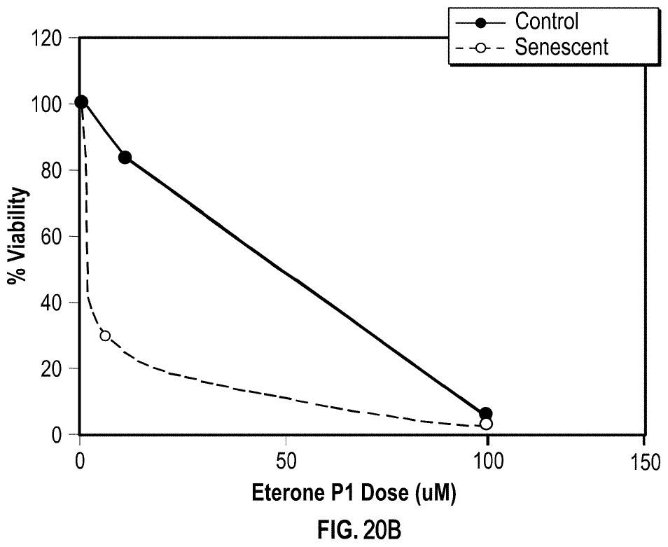

WI-38 cells (ATCC #CCL-75, a diploid primary human fibroblast adherent cell line derived from fetal lung tissue) and IMR90 cells (ATCC #CCL-186, a diploid primary human fibroblast adherent cell line derived from fetal lung tissue) were treated with a chemotherapy agent, 100 NM Doxorubicin twice every other day to induce senescence. After 5 days, the cells were assayed for Senescence-Associated B-Galactosidase (SA-B-Gal) activity (Senescence Detection Kit, Abcam) and confirmed to possess SA-B-Gal activity. Later these cells were used for experiments comparing them to their non-Doxorubicin (non-senescent) treated counterparts. Senescent and non-senescent WI-38/IMR90 fibroblasts were plated for XTT viability assays. 7000 senescent (obtained as described above) and 2000 non-senescent WI-38/IMR90 fibroblasts were plated in 96-well plates incubated. The cells were treated with mock (PBS) or the Senolytic Peptide (SEQ ID NO: 12) at total doses ranging from 0 to 100 pM. The mock and peptide treatments were carried out for 3 consecutive days (24, 48 and 72 hours) after plating. After treatment period, the cells were incubated for 7 days until XTT-based viability was analyzed according to the manufacturer's instructions. The senescent cells showed much steeper decrease in viability compared with the non-senescent cells. The Senolytic Peptide (SEQ ID NO: 12) was ordered from Chinese Peptide Company with TFA removal. The Senolytic Peptide (SEQ ID NO: 12) selectively decreased the viability of senescent WI-38 (FIG. 20A) and IMR-90 (FIG. 20B) cells but not non-senescent cell counterparts.

The following embodiments demonstrate additional aspects of to disclosed subject matter.

A first embodiment is an artificial peptide comprises an amino acid sequence having at least 90% identify to any one of SEQ ID NO: 1 through SEQ ID NO: 245.

A second embodiment is the artificial peptide according to the first embodiment, wherein the amino acid sequence has at least 95% identify to any one of SEQ ID NO: 1 through SEQ ID NO: 245.

A third embodiment is the artificial peptide according to any one of the first through the second embodiments, wherein the amino acid sequence has at least 98% identify to any one of SEQ ID NO: 1 through SEQ ID NO: 245.

A fourth embodiment is the artificial peptide according to any one of the first through the third embodiments, wherein the amino acid sequence comprises any one of SEQ ID NO: 1 through SEQ ID NO: 245.

A fifth embodiment is the artificial peptide according to any one of the first through the fourth embodiments, wherein the amino acid sequence has at least 90% identify to any one of SEQ ID NO: 1 through SEQ ID NO: 12.

A sixth embodiment is the artificial peptide according to any one of the first through the fifth embodiments, wherein the amino acid sequence has at least 95% identify to any one of SEQ ID NO: 1 through SEQ ID NO: 12.

A seventh embodiment is the artificial peptide according to any one of the first through the sixth embodiments, wherein the amino acid sequence comprises any one of SEQ ID NO: 1 through SEQ ID NO: 12.

An eighth embodiment is the artificial peptide according to any one of the first through the seventh embodiments, wherein the artificial peptide further comprises an N- terminal amino acid sequence that facilitates cellular uptake or a C-terminal amino acid sequence that facilitates cellular uptake.

A ninth embodiment is the artificial peptide according to any one of the first through the eighth embodiments, wherein the artificial peptide further comprises an N- terminus amino acid sequence comprising a single D-amino acid.

A tenth embodiment is the artificial peptide according to any one of the first through the ninth embodiments, wherein the artificial peptide exhibits a circular structure.

An eleventh embodiment is a composition comprising the artificial peptide according to any one of the first through the tenth embodiments.

A twelfth embodiment is a method of inducing the apoptosis of a senescent cell in a subject, the method comprising causing an artificial peptide comprising an amino acid sequence having at least 90% identify to any one of SEQ ID NO: 1 through SEQ ID NO: 245 to interfere with the CR3 domain of Forkhead box protein 04 (FoxO4) of the senescent cell.

A thirteenth embodiment is the method according to the twelfth embodiment, wherein the amino acid sequence has at least 95% identify to any one of SEQ ID NO: 1 through SEQ ID NO: 245.

A fourteenth embodiment is the method according to any one of the twelfth through the thirteenth embodiments, wherein the amino acid sequence has at least 95% identify to any one of SEQ ID NO: 1 through SEQ ID NO: 245.

A fifteenth embodiment is the method according to any one of the twelfth through the fourteenth embodiments, wherein the amino acid sequence comprises any one of SEQ ID NO: 1 through SEQ ID NO: 245.

A sixteenth embodiment is the method according to any one of the twelfth through the fifteenth embodiments, wherein the amino acid sequence has at least 90% identify to any one of SEQ ID NO: 1 through SEQ ID NO: 12.

A seventeenth embodiment is the method according to any one of the twelfth through the sixteenth embodiments, wherein the amino acid sequence has at least 95% identify to any one of SEQ ID NO: 1 through SEQ ID NO: 12.

A eighteenth embodiment is the method according to any one of the twelfth through the seventeenth embodiments, wherein the amino acid sequence comprises any one of SEQ ID NO: 1 through SEQ ID NO: 12.

A ninteenth embodiment is the method according to any one of the twelfth through the eighteenth embodiments, wherein the artificial peptide further comprises an N-terminal amino acid sequence that facilitates cellular uptake or a C-terminal amino acid sequence that facilitates cellular uptake.

A twentieth embodiment is the method according to any one of the twelfth through the ninteenth embodiments, wherein the artificial peptide further comprises an N- terminus amino acid sequence comprising a single D-aa.

A twenty-first embodiment is the method according to any one of the twelfth through the twentieth embodiments, wherein the artificial peptide exhibits a circular structure.

A twenty-second embodiment is the method according to any one of the twelfth through the twenty-first embodiments, wherein causing the artificial peptide to interfere with the CR3 domain of FoxO4 of the senescent cell comprises administering a pharmaceutical composition comprising the artificial peptide according to any one of claims 12-21 to the subject.

A twenty-third embodiment is the method according to any one of the twelfth through the twenty-second embodiments, wherein the artificial peptide exhibits maximized interference with the CR3 domain of FoxO4.

A twenty-fourth embodiment is the method according to any one of the twelfth through the twenty-third embodiments, wherein the artificial peptide exhibits reduced direct interaction with the p53DBD compared to that of the endogenous FoxO4.

A twenty-fifth embodiment is the method according to any one of the twelfth through the twenty-fourth embodiments, wherein the artificial peptide preferably exhibits a reduced interference with the CR3 domains of FoxO1 or FoxO3 compared to that of said FoxO4.

A twenty-sixth embodiment is the method according to any one of the twelfth through the twenty-fifth embodiments, wherein the artificial peptide exhibits reduced interference with DNA compared to that of said FoxO4.

A twenty-seventh embodiment is the method according to any one of the twelfth through the twenty-sixth embodiments, wherein the senescent cell is characterized as expressing the senescence-associated secretory phenotype (SASP).

A twenty-eighth embodiment is the method according to any one of the twelfth through the twenty-seventh embodiments, wherein the method comprises treatment of a senescence-associated disease or disorder.

A twenty-ninth embodiment is the method according the twenty-eighth embodiment, wherein the disease or disorder is cancer, and wherein the subject is a mammal, preferably a human, and wherein the artificial peptide is administered before, during, and/or after subjecting the subject to radiation therapy, and/or before, during or after administering to the subject at least one chemotherapeutic agent.

A thirtieth embodiment is the method according to any one of the twenty-eighth through the twenty-ninth embodiments, wherein the said cancer is characterized as resistant to therapy.

A thirty-first embodiment is the method according to any one of the twenty-eighth through the thirtieth embodiments, wherein said therapy-resistant cancer comprises is metastatic melanoma, breast cancer, or glioblastoma, and wherein the therapy to which the cancer is resistant is radiation therapy or chemotherapy.

A thirty-second embodiment is the method according to any one of the twelfth through the thirty-first embodiments, wherein the subject comprises a human characterized as suffering from, or expected to suffer from chronic inflammatory diseases or a senescence related disease or disorder.

A thirty-third embodiment is the method according to any one of the twelfth through the thirty-second embodiments, wherein the method is effective to remove cells from the subject that express p16INK4a, wherein the subject is characterized as suffering from, or expected to suffer from a senescence-associated disease or disorder.

A thirty-fourth embodiment is the method according to any one of the twelfth through the thirty-third embodiments, wherein the method is effective to alter levels of the Serine-46 phosphorylated p53 foci in the subject, wherein the subject is characterized as suffering from, or expected to suffer from, a senescence-associated disease or disorder.

A thirty-fifth embodiment is the method according to any one of the twelfth through the thirty-fourth embodiments, wherein the method comprises administering the artificial peptide according to an Impulse Regime, Sustained Regime, a Gentle Shock Regime, or combinations thereof.

As will be recognized by those skilled in the art, the Senolytic Peptide(s) and the disclosed method described in the present application can be modified and varied over a tremendous range of applications to produce a wide range of senolytic peptides which may be directed to specific therapies which target those subsets of senescent cells and accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

1. An artificial peptide comprising an amino acid sequence having at least 90% identify to any one of SEQ ID NO: 1 through SEQ ID NO: 245.

2. The artificial peptide according to claim 1, wherein the amino acid sequence has at least 95% identify to any one of SEQ ID NO: 1 through SEQ ID NO: 245.

3. The artificial peptide according to claim 2, wherein the amino acid sequence has at least 98% identify to any one of SEQ ID NO: 1 through SEQ ID NO: 245.

4. The artificial peptide according to claim 1, wherein the amino acid sequence comprises any one of SEQ ID NO: 1 through SEQ ID NO: 245.

5. The artificial peptide according to claim 2, wherein the amino acid sequence is any one of SEQ ID NO: 1 through SEQ ID NO: 12.

6. The artificial peptide according to claim 3, wherein the amino acid sequence is any one of SEQ ID NO: 1 through SEQ ID NO: 12.

7. The artificial peptide according to claim 6, wherein the amino acid sequence comprises any one of SEQ ID NO: 1 through SEQ ID NO: 12.

8. The artificial peptide according to claim 1, wherein the artificial peptide further comprises an N-terminal amino acid sequence that facilitates cellular uptake or a C-terminal amino acid sequence that facilitates cellular uptake.

9. The artificial peptide according to claim 1, wherein the artificial peptide further comprises an N-terminus amino acid sequence comprising a single D-amino acid.

10. The artificial peptide according to claim 1, wherein the artificial peptide exhibits a circular structure.

11. (canceled)

12. A method of inducing the apoptosis of a senescent cell in a subject, the method comprising causing an artificial peptide comprising an amino acid sequence having at least 90% identify to any one of SEQ ID NO: 1 through SEQ ID NO: 245 to interfere with the CR3 domain of Forkhead box protein 04 (FoxO4) of the senescent cell.

13. The method according to claim 12, wherein the amino acid sequence has at least 95% identify to any one of SEQ ID NO: 1 through SEQ ID NO: 245.

14. The method according to claim 13, wherein the amino acid sequence has at least 98% identify to any one of SEQ ID NO: 1 through SEQ ID NO: 245.

15. The method according to claim 14, wherein the amino acid sequence comprises any one of SEQ ID NO: 1 through SEQ ID NO: 245.

16. The method according to claim 14, wherein the amino acid sequence any one of SEQ ID NO: 1 through SEQ ID NO: 12.

17. The method according to claim 16, wherein the amino acid sequence has at least 95% identify to any one of SEQ ID NO: 1 through SEQ ID NO: 12.

18. The method according to claim 17, wherein the amino acid sequence comprises any one of SEQ ID NO: 1 through SEQ ID NO: 12.

19. The method according to claim 12, wherein the artificial peptide further comprises an N-terminal amino acid sequence that facilitates cellular uptake or a C-terminal amino acid sequence that facilitates cellular uptake.

20. The method according to claim 12, wherein the artificial peptide further comprises an N-terminus amino acid sequence comprising a single D-aa.

21. The method according to claim 12, wherein the artificial peptide exhibits a circular structure.

22. The method according to claim 12, wherein causing the artificial peptide to interfere with the CR3 domain of FoxO4 of the senescent cell comprises administering a pharmaceutical composition comprising the artificial peptide the subject.

23. The method according to claim 22, wherein the artificial peptide exhibits maximized interference with the CR3 domain of FoxO4.

24. The method according to claim 22, wherein the artificial peptide exhibits reduced direct interaction with the p53DBD compared to that of the endogenous Foxo4.

25. The method according to claim 22, wherein the artificial peptide preferably exhibits a reduced interference with the CR3 domains of FoxO1 or FoxO3 compared to that of said FoxO4.

26. The method according to claim 22, wherein the artificial peptide exhibits reduced interference with DNA compared to that of said FoxO4.

27. The method according to claim 22, wherein the senescent cell is characterized as expressing the senescence-associated secretory phenotype (SASP).

28. The method according to claim 22, wherein the method comprises treatment of a senescence-associated disease or disorder.

29. The method according claim 28, wherein the disease or disorder is cancer, and wherein the subject is a mammal, preferably a human, and wherein the artificial peptide is administered before, during, and/or after subjecting the subject to radiation therapy, and/or before, during or after administering to the subject at least one chemotherapeutic agent.

30. The method according to claim 29, wherein the said cancer is characterized as resistant to therapy.

31. The method according to claim 30, wherein said therapy-resistant cancer comprises is metastatic melanoma, breast cancer, or glioblastoma, and wherein the therapy to which the cancer is resistant is radiation therapy or chemotherapy.

32. The method according to claim 12, wherein the subject comprises a human characterized as suffering from, or expected to suffer from chronic inflammatory diseases or a senescence related disease or disorder.

33. The method according to claim 12, wherein the method is effective to remove cells from the subject that express p16INK4a, wherein the subject is characterized as suffering from, or expected to suffer from a senescence-associated disease or disorder.

34. The method according to claim 12, wherein the method is effective to alter levels of the Serine-46 phosphorylated p53 foci in the subject, wherein the subject is characterized as suffering from, or expected to suffer from, a senescence-associated disease or disorder.

35. The method according to claim 12, wherein the method comprises administering the artificial peptide according to an Impulse Regime, Sustained Regime, a Gentle Shock Regime, or combinations thereof.
